SEQUENCE LISTING (72 sequences)

SEQ ID NO: 1 - DNA, 21 bp, Artificial Sequence
  Description of Artificial Sequence Synthetic primer
tgggtgggtccatggggaaac

SEQ ID NO: 2 - DNA, 20 bp, Artificial Sequence
  Description of Artificial Sequence Synthetic primer
gcttctgctctgttcccagt

SEQ ID NO: 3 - DNA, 18 bp, Artificial Sequence
  Description of Artificial Sequence Synthetic primer
aggccctggaggagacag

SEQ ID NO: 4 - DNA, 20 bp, Artificial Sequence
  Description of Artificial Sequence Synthetic primer
caggggtcctaccactcagg

SEQ ID NO: 5 - DNA, 21 bp, Artificial Sequence
  Description of Artificial Sequence Synthetic primer
acgcttgtgtttctcagtccg

SEQ ID NO: 6 - DNA, 20 bp, Artificial Sequence
  Description of Artificial Sequence Synthetic primer
tggtctagttcccgcctcct

SEQ ID NO: 7 - DNA, 20 bp, Artificial Sequence
  Description of Artificial Sequence Synthetic primer
agcaggctgtggctctgatt

SEQ ID NO: 8 - DNA, 20 bp, Artificial Sequence
  Description of Artificial Sequence Synthetic primer
ccagcctcttctatgccaga

SEQ ID NO: 9 - DNA, 25 bp, Artificial Sequence
  Description of Artificial Sequence Synthetic primer
ttgttcaatgtatccaaaagaaaca

SEQ ID NO: 10 - DNA, 26 bp, Artificial Sequence
  Description of Artificial Sequence Synthetic primer
tgagataaagcttcttcccttaaaaa

SEQ ID NO: 11 - DNA, 20 bp, Artificial Sequence
  Description of Artificial Sequence Synthetic primer
cataagcctgcgtcagatca

SEQ ID NO: 12 - DNA, 20 bp, Artificial Sequence
  Description of Artificial Sequence Synthetic primer
cctgtgttgggttgacagtg

SEQ ID NO: 13 - DNA, 22 bp, Artificial Sequence
  Description of Artificial Sequence Synthetic primer
gggtattgggaatagagtagaa

SEQ ID NO: 14 - DNA, 21 bp, Artificial Sequence
  Description of Artificial Sequence Synthetic primer
cacccttcaaaaaaataccta

SEQ ID NO: 15 - DNA, 29 bp, Artificial Sequence
  Description of Artificial Sequence Synthetic primer
atttctagaaattcttggctggacatggt

SEQ ID NO: 16 - DNA, 31 bp, Artificial Sequence
  Description of Artificial Sequence Synthetic primer
atttctagatcaaagtgagaaaatgcctcaa

SEQ ID NO: 17 - DNA, 29 bp, Artificial Sequence
  Description of Artificial Sequence Synthetic primer
atttctagattcccagcctaggtttcaga

SEQ ID NO: 18 - DNA, 29 bp, Artificial Sequence
  Description of Artificial Sequence Synthetic primer
atttctagatgagatgcgatcaaacatcc

SEQ ID NO: 19 - DNA, 30 bp, Artificial Sequence
  Description of Artificial Sequence Synthetic primer
atttctagacaataaatggccaaagggatt

SEQ ID NO: 20 - DNA, 29 bp, Artificial Sequence
  Description of Artificial Sequence Synthetic primer
atttctagacttcaagctgcccagtgata

SEQ ID NO: 21 - DNA, 30 bp, Artificial Sequence; identical to SEQ ID NO: 19
  Description of Artificial Sequence Synthetic primer

SEQ ID NO: 22 - DNA, 29 bp, Artificial Sequence
  Description of Artificial Sequence Synthetic primer
atttctagacaaaagctagtccccgtctg

SEQ ID NO: 23 - DNA, 29 bp, Artificial Sequence
  Description of Artificial Sequence Synthetic primer
atttctagaaattcttggctggacatggt

SEQ ID NO: 24 - DNA, 29 bp, Artificial Sequence
  Description of Artificial Sequence Synthetic primer
atttctagacaaaagctagtccccgtctg

SEQ ID NO: 25 - DNA, 30 bp, Artificial Sequence
  Description of Artificial Sequence Synthetic primer
atttctagaggaaagctgaaagatgcactg

SEQ ID NO: 26 - DNA, 29 bp, Artificial Sequence
  Description of Artificial Sequence Synthetic primer
atttctagaggagccacagagcatgagat

SEQ ID NO: 27 - DNA, 29 bp, Artificial Sequence
  Description of Artificial Sequence Synthetic primer
atttctagaatccattcacaagcctcctg

SEQ ID NO: 28 - DNA, 29 bp, Artificial Sequence
  Description of Artificial Sequence Synthetic primer
atttctagaccttcccatctgtgtccttg

SEQ ID NO: 29 - DNA, 29 bp, Artificial Sequence
  Description of Artificial Sequence Synthetic primer
atttctagattttgctggtcagcagtttg

SEQ ID NO: 30 - DNA, 29 bp, Artificial Sequence
  Description of Artificial Sequence Synthetic primer
atttctagatccatctgcacagaagcagt

SEQ ID NO: 31 - DNA, 44 bp, Artificial Sequence
  Description of Artificial Sequence Synthetic primer
ttaattgaccatactggatactatttctgttctctcttccccaa

SEQ ID NO: 32 - DNA, 44 bp, Artificial Sequence
  Description of Artificial Sequence Synthetic primer
ttggggaagagagaacagaaatagtatccagtatggtcaattaa

SEQ ID NO: 33 - DNA, 40 bp, Artificial Sequence
  Description of Artificial Sequence Synthetic primer
tgtacacacccgcctgacaccattacaaaaaaacacgtgg

SEQ ID NO: 34 - DNA, 40 bp, Artificial Sequence
  Description of Artificial Sequence Synthetic primer
ccacgtgtttttttgtaatggtgtcaggcgggtgtgtaca

SEQ ID NO: 35 - DNA, 40 bp, Artificial Sequence
  Description of Artificial Sequence Synthetic primer
tttctctgttcctaggactcttacagttctatgttagacc

SEQ ID NO: 36 - DNA, 40 bp, Artificial Sequence
  Description of Artificial Sequence Synthetic primer
ggtctaacatagaactgtaagagtcctaggaacagagaaa

SEQ ID NO: 37 - DNA, 30 bp, Artificial Sequence
  Description of Artificial Sequence Synthetic primer
attgagctccctacgacacagtgcgagagg

SEQ ID NO: 38 - DNA, 30 bp, Artificial Sequence
  Description of Artificial Sequence Synthetic primer
actctcgagggaaaccaaggcacagaggaa

SEQ ID NO: 39 - DNA, 30 bp, Artificial Sequence
  Description of Artificial Sequence Synthetic primer
attgagctccagcccccctctcagatcctc

SEQ ID NO: 40 - DNA, 30 bp, Artificial Sequence
  Description of Artificial Sequence Synthetic primer
attgagctcatcagggcacagggggaccca

SEQ ID NO: 41 - DNA, 30 bp, Artificial Sequence
  Description of Artificial Sequence Synthetic primer
attgagctcctctgggctctgccttgcccc

SEQ ID NO: 42 - DNA, 30 bp, Artificial Sequence
  Description of Artificial Sequence Synthetic primer
attgagctcccagctccagcacttggaggg

SEQ ID NO: 43 - DNA, 30 bp, Artificial Sequence
  Description of Artificial Sequence Synthetic primer
attgagctcattgccccccacacatcttgt

SEQ ID NO: 44 - DNA, 29 bp, Artificial Sequence
  Description of Artificial Sequence Synthetic primer
attgagctcccctgccctgcttcccagtg

SEQ ID NO: 45 - DNA, 30 bp, Artificial Sequence
  Description of Artificial Sequence Synthetic primer
attgagctcgaaaccaaggctcgggttggg

SEQ ID NO: 46 - DNA, 30 bp, Artificial Sequence
  Description of Artificial Sequence Synthetic primer
attgagctcgtgggggagatctgggtactg

SEQ ID NO: 47 - DNA, 30 bp, Artificial Sequence
  Description of Artificial Sequence Synthetic primer
attgagctcggacagctggggcagcaggct

SEQ ID NO: 48 - DNA, 31 bp, Artificial Sequence
  Description of Artificial Sequence Synthetic primer
attgagctctcctggacccgccccaccctgc

SEQ ID NO: 49 - DNA, 44 bp, Artificial Sequence
  Description of Artificial Sequence Synthetic primer
ccagcctgatgcttcctggatcctccccaccctgcccgggcaca

SEQ ID NO: 50 - DNA, 44 bp, Artificial Sequence
  Description of Artificial Sequence Synthetic primer
tgtgcccgggcagggtggggaggatccaggaagcatcaggctgg

SEQ ID NO: 51 - DNA, 44 bp, Artificial Sequence
  Description of Artificial Sequence Synthetic primer
gcttcctggacccgccccactcttcccgggcacagtccagggct

SEQ ID NO: 52 - DNA, 44 bp, Artificial Sequence
  Description of Artificial Sequence Synthetic primer
agccctggactgtgcccgggaagagtggggcgggtccaggaagc

SEQ ID NO: 53 - DNA, 44 bp, Artificial Sequence
  Description of Artificial Sequence Synthetic primer
gcttcctggatcctccccactcttcccgggcacagtccagggct

SEQ ID NO: 54 - DNA, 44 bp, Artificial Sequence
  Description of Artificial Sequence Synthetic primer
agccctggactgtgcccgggaagagtggggaggatccaggaagc

SEQ ID NO: 55 - DNA, 34 bp, Artificial Sequence
  Description of Artificial Sequence Synthetic oligonucleotide
cttcctggacccgccccaccctgcccgggcacag

SEQ ID NO: 56 - RNA, 21 nt, Homo sapiens
cugaccuaugaauugacagcc

SEQ ID NO: 57 - RNA, 23 nt, Homo sapiens
accauacuggauacuuaggucaa

SEQ ID NO: 58 - RNA, 21 nt, Homo sapiens
augaccuaugaauugacagac

SEQ ID NO: 59 - RNA, 20 nt, Homo sapiens
aggcuguauuccggggucaa

SEQ ID NO: 60 - DNA, 21 bp, Homo sapiens
ccgcctgacaccgtgggtcat

SEQ ID NO: 61 - DNA, 22 bp, Homo sapiens
cccaggctggagtgcagtggcg

SEQ ID NO: 62 - RNA, 22 nt, Homo sapiens
uguaacagcaacuccaugugga

SEQ ID NO: 63 - DNA, 21 bp, Homo sapiens
gtgcaattctcaaaaggttag

SEQ ID NO: 64 - RNA, 21 nt, Homo sapiens
cugaccuaugaauugacagcc

SEQ ID NO: 65 - DNA, 22 bp, Homo sapiens
ttgatatatggaggcagtgaca

SEQ ID NO: 66 - RNA, 22 nt, Homo sapiens
uguaacagcaacuccaugugga

SEQ ID NO: 67 - DNA, 21 bp, Homo sapiens
ccctgtcttctcttaggtcac

SEQ ID NO: 68 - RNA, 21 nt, Homo sapiens
cugaccuaugaauugacagcc

SEQ ID NO: 69 - DNA, 21 bp, Homo sapiens
ccgcctgacaccgtgggtcat

SEQ ID NO: 70 - RNA, 21 nt, Homo sapiens
augaccuaugaauugacagac

SEQ ID NO: 71 - RNA, 20 nt, Homo sapiens
aggcuguauuccggggucaa

SEQ ID NO: 72 - RNA, 21 nt, Homo sapiens
mugaccuaugaauugacagcc

(19) United States
(12) Patent Application Publication
(10) Pub. No.: US 2013/0150430 A1
(43) Pub. Date: Jun. 13, 2013

(54) Methods for Impairing the P53/HDM2 Auto-Regulatory Loop in Multiple Myeloma Development Using mIR-192, mIR-194 and mIR-215

(71) Applicants: Carlo M. Croce, Columbus, OH (US); Flavia Pichiorri, Columbus, OH (US)

(72) Inventors: Carlo M. Croce, Columbus, OH (US); Flavia Pichiorri, Columbus, OH (US)

(73) Assignee: THE OHIO STATE UNIVERSITY, Columbus, OH (US)

(21) Appl. No.: 13/813,773

(22) PCT Filed: Aug. 4, 2011
(86) PCT No.: PCT/US11/46663

§ 371 Date: Feb. 1, 2013

(51) Int. Cl. A61K 31/7088; A61K 31/496; A61K 31/407

(52) U.S. Cl. CPC A61K 31/7088; A61K 31/407; A61K 31/496
USPC 514/44 A; 435/375; 506/9; 435/29; 435/6.11

(57) ABSTRACT

Methods and compositions for detecting, treating, characterizing, and diagnosing multiple myeloma are described.

### CROSS-REFERENCE TO RELATED APPLICATIONS

This application claims the benefit of U.S. Provisional Application Ser. No. 61/370,692 filed Aug. 4, 2010, the entire disclosure of which is expressly incorporated herein by reference.

### STATEMENT REGARDING FEDERALLY SPONSORED RESEARCH

This invention was not made with government support and the government has no rights in the invention.

### SEQUENCE LISTING

The instant application contains a Sequence Listing which has been submitted via EFS-web and is hereby incorporated by reference in its entirety. The ASCII copy, created on Aug. 4, 2011, is named 604_{—}52261_SEQ_LIST_{—}11011.txt, and is 15,214 bytes in size.

### TECHNICAL FIELD AND INDUSTRIAL APPLICABILITY OF THE INVENTION

This invention relates generally to the field of molecular biology. More particularly, it concerns cancer-related technology. Certain aspects of the invention include application in diagnostics, therapeutics, and prognostics of multiple myeloma (MM).

### BACKGROUND OF THE INVENTION

Multiple myeloma (MM) is a plasma cell proliferative disorder that results in considerable morbidity and mortality. As it is incurable with the current therapeutic approaches, more effective therapies based on better understanding of the patho-biology of the disease are needed. This cancer develops from a benign condition called monoclonal gammopathy of undetermined significance (MGUS). Individuals with MGUS often remain stable for years and do not require treatment. However, for unknown reasons, this benign condition can evolve into MM at a rate of ˜1% per year, with some MMs developing after many years.

The tumor suppressor, p53, is a powerful anti-tumoral protein frequently inactivated by mutations or deletions in cancer. p53 is a potent transcription factor that is activated in response to diverse stresses, leading to induction of cell-cycle arrest, apoptosis or senescence. Although regulation of the p53 pathway is not fully understood at the molecular level, it has been well established that activated p53 is detrimental to cancer progression, underlining why cancer cells have developed multiple mechanisms for disabling p53 function. Half of human tumors retain wild-type (WT) p53, and its up-regulation, by antagonizing its negative regulator, human double minute 2 (HDM2), offers a therapeutic strategy.

Hematological cancers such as multiple myeloma (MM), acute myeloid leukemia, chronic lymphocytic leukemia and Hodgkin's disease (HD) are important models in the study of endogenous p53 reactivation; in these cancers, TP53 gene mutations are rarely detected at diagnosis, although their prevalence may increase with progression to more aggressive or advanced stages.

In MGUS and in the majority of new diagnosed MM cases TP53 is WT and the protein is rarely detectable. Interestingly, in MM cells, expression of p53 protein levels can be rescued by antagonizing MDM2.

Micro-RNAs representing between 1% and 3% of all eukaryotic genes, are a class of endogenous noncoding RNAs, 19-25 nt in size, which regulate gene expression at the transcriptional or translational level. Approximately half of human microRNAs are located at fragile sites and genomic regions involved in alterations in cancers, and alteration of microRNA expression profiles occurs in most cancers, suggesting that individual microRNAs could function as tumor suppressors or oncogenes.

### SUMMARY OF THE INVENTION

The present invention is based, at least in part, on the inventors' discoveries, using small-molecule inhibitors of MDM2 (murine double minute2), that miR-192, miR-194 and miR-215, which are down-regulated in a subset of newly diagnosed multiple myeloma (MM) subjects, are transcriptionally activated by p53 and then modulate MDM2 expression.

Further, the inventors herein have discovered that ectopic re-expression of these miRNAs in MM cells is crucial for full p53 activation, increasing the therapeutic action of MDM2 inhibitors in vitro and in vivo.

In addition, miR-192 and miR-215 target the insulin growth factor axis (IGF axis), preventing enhanced migration of plasma cells into bone marrow.

The inventors herein also show that these miRNAs are positive regulators of p53 and that their down-regulation plays a key role in MM development.

In a broad aspect, there is provided herein a method of treating a disorder mediated by a p53-HDM2 interaction comprising administering to a subject in need thereof a combination of at least miR gene product and at least one indole inhibitor of human double minute 2 (HDM2), or a pharmaceutically acceptable salt, solvate, or prodrug thereof.

In certain embodiments, wherein the disorder is multiple myeloma, and the miR gene product comprises one or more of: miR-192, miR-194 and miR-215.

In another broad aspect, the invention herein relates to a combination of an indole inhibitor of human double minute 2 (HDM2), or a pharmaceutically acceptable salt, solvate, or prodrug thereof, and one or more of a miR gene product selected from: miR-192, miR0194 and miR-215.

In certain embodiments, the indole inhibitors of human double minute 2 (HDM2) can be one or more of the compositions as described in the Wang et al. U.S. Pat. No. 7,737,174; the Wang et al. U.S. Pat. No. 7,759,383, the Wang et al. US Pub. No. 2010/0317661; and the Wang et al. US Pub. No. 2011/0112052. One exemplary indole inhibitor of HDM2 is known as MI-219, having the structure

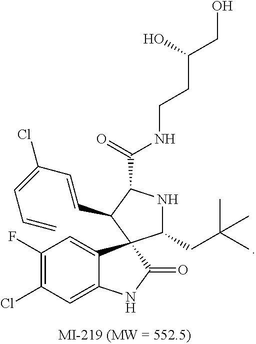 [SMILES: C=C/C=C(Cl)\C=C\[C@H]1[C@H](C(=O)NCC[C@H](O)CO)N[C@H](CC(C)(C)C)[C@]12C(=O)Nc1cc(Cl)c(F)cc12]

In other embodiments, the indole inhibitor of human double minute 2 (HDM2) can comprises a Nutlin, such as Nutlin 3, having the structure

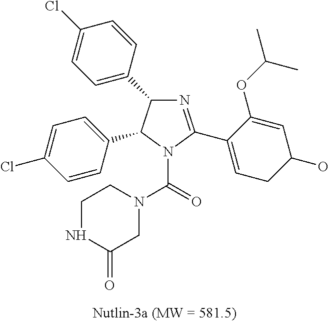 [SMILES: CC(C)OC1=CC(O)CC=C1C1=N[C@@H](c2ccc(Cl)cc2)[C@@H](c2ccc(Cl)cc2)N1C(=O)N1CCNC(=O)C1]

In another broad aspect, the invention herein relates to a pharmaceutical composition comprising the combination as described herein.

In another broad aspect, the invention herein relates to a commercial package comprising a combination as described herein. In certain embodiments, the commercial package includes a unit dosage form is a fixed combination.

In another broad aspect, the invention herein relates to a method of treating a subject comprising administering to the subject a therapeutically effective amount of the combination as described herein, wherein the subject has a hyperproliferative disease. In certain embodiments, the hyperproliferative disease is multiple myeloma. Also, in certain embodiments, cells of the hyperproliferative disease express functional p53.

In another broad aspect, the invention herein relates to a kit comprising a combination of claim 6, and instructions for administering the compound to a subject having a hyperproliferative disease.

In certain embodiments, the hyperproliferative disease is multiple myeloma.

In certain embodiments, the instructions direct co-administration of the compound together with the one or more anticancer agents.

In another broad aspect, the invention herein relates to a method of treating a disorder in a subject, comprising administering to said subject a therapeutically effective amount of a combination of claim 3, claim 4 or claim 5, wherein the disorder is multiple myeloma.

In certain embodiments, the indole inhibitor of human double minute 2 (HDM2) is administered prior to the miR gene product.

In certain embodiments, the indole inhibitor of human double minute 2 (HDM2) is administered after to the miR gene product.

In certain embodiments, the indole inhibitor of human double minute 2 (HDM2) is administered concurrently with the miR gene product.

In another broad aspect, the invention herein relates to a combination of: i) an indole inhibitor of human double minute 2 (HDM2); and ii) a miR gene product comprising one or more of: miR-192, miR-194 and miR-215; for simultaneous, concurrent, separate or sequential use in for preventing or treating a proliferative disease.

In certain embodiments, the indole inhibitor of human double minute 2 (HDM2) comprises MI-219 or of a pharmaceutically acceptable salt, ester or prodrug thereof.

In certain embodiments, the indole inhibitor of human double minute 2 (HDM2) comprises Nutlin 3 or of a pharmaceutically acceptable salt, ester or prodrug thereof.

In another broad aspect, the invention herein relates to a pharmaceutical composition comprising the combination as described herein.

In another broad aspect, the invention relates to a commercial package comprising the combination as described herein. In certain embodiments, the A commercial package includes a unit dosage form in a fixed combination.

In another broad aspect, the invention herein relates to a method of treating in a subject a disorder mediated by a p53-MDM2 interaction comprising administering to the subject a therapeutically effective amount of a pharmaceutical composition comprising a combination of i) an indole inhibitor of human double minute 2 (HDM2); and ii) a miR gene product comprising one or more of: miR-192, miR-194 and miR-215; and a pharmaceutically acceptable carrier.

In another broad aspect, the invention herein relates to a method for regulating human double minute 2 (HDM2)-p53 auto regulatory loop, in a subject in need thereof, comprising upregulating the expression of one or more of: miR-192, miR-194 and miR-215.

In another broad aspect, the invention herein relates to a method for increasing the ability of p53 to modulate HDM2 expression in a subject having multiple myeloma (MM), comprising administering an effective amount of a miR gene product comprising one or more of: miR-192, miR-194 and miR-215, sufficient to inhibit expression of HDM2.

In another broad aspect, the invention herein relates to a use of miR-192, miR-194 and/or miR-215 as mediators in the pharmacological activation of the p53 pathway in multiple myeloma (MM) cells.

In another broad aspect, the invention herein relates to a method for inhibiting expression of HDM2 mRNA comprising up-modulating expression of one or more of: miR-192, miR-194 and miR-215.

In another broad aspect, the invention herein relates to a composition for inhibiting cell growth and enhancing apoptosis in multiple myeloma cells, comprising a gene product comprising one or more of: miR-192, miR-194 and miR-215.

In certain embodiments, the composition further includes one or more HDM2 inhibitors.

In certain embodiments, the HDM2 inhibitor comprises MI-219.

In certain embodiments, the HDM2 inhibitor comprises Nutlin 3a.

In another broad aspect, the invention herein relates to a method for inhibiting cell growth and enhancing apoptosis in multiple myeloma (MM) cells, comprising administering:

an effective amount of one or more miR gene products that affect proliferation rate in MM cells and/or the homing and migration ability of MM cells,

wherein the miR gene products comprises one or more of: miR-192, miR-194 and miR-215.

In certain embodiments, the method further includes administering one or more p53 pharmacological activators in an amount sufficient to cause HDM2 down-regulation, and/or one or more of: p53, p21, Puma up-regulation.

In another broad aspect, the invention herein relates to a method of treating multiple myeloma (MM) in a subject who has a MM in which at least one miR gene product is down-regulated in the MM cells of the subject relative to control cells, comprising:

administering to the subject an effective amount of at least one isolated miR gene product, wherein the miR gene product comprises one or more of: miR-192, miR-194 and miR-215, such that proliferation of MM cells in the subject is inhibited.

In certain embodiments, the method further includes administering an effective amount of a p53 pharmacological activator. In certain embodiments, the p53 pharmacological activator comprises one or more of: MI-219 and Nutlin 3.

In another broad aspect, the invention herein relates to a pharmaceutical composition for treating MM, comprising at least one isolated miR gene product and a pharmaceutically-acceptable carrier, wherein the at least one isolated miR gene product corresponds to a miR gene product that is down-regulated in MM cells relative to suitable control cells, wherein the isolated miR gene product comprises one or more of: miR-192, miR194 and miR-215.

In another broad aspect, the invention herein relates to a method of diagnosing multiple myeloma, comprising detecting an increased amount of one or more of: miR-192, miR-194 and miR-215 genes as compared to a control.

In another broad aspect, the invention herein relates to a method of identifying an anti-MM agent, comprising providing a test agent to a cell and measuring the level of at least one miR gene product associated with decreased expression levels in MM cells, wherein an increase in the level of the miR gene product in the cell, relative to a suitable control cell, is indicative of the test agent being an anti-MM agent.

Other systems, methods, features, and advantages of the present invention will be or will become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the Patent Office upon request and payment of the necessary fee.

FIGS. 1A-1D. Identification of p53-regulated miRNAs in MM cells:

FIG. 1A: Overview of two-way (genes against samples) hierarchical cluster (Euclidean distance) of 6 MM cell lines in duplicate using the genes that vary the most between samples. As shown, the clustering is mainly determined by the presence of WT TP53 expression (NCI-H929, MM1s and KMS28BM) or mutant/null TP53 (U266, RPMI-8226, JJN3) in the cell lines. In magnification are reported the miRNAs up-regulated more than 3-fold in WT TP53 cell lines with P <0.001.

FIG. 1B: Overview of two-way of MM1s cells treated with 10 μM Nutlin-3a overnight (biological quadruplicate) and with DMSO (biological triplicate) using the genes that vary the most between samples. As shown, the clustering is mainly determined from the Nutlin-3a treatments and DMSO treatment. Color areas indicate relative expression of each gene with respect to the gene median expression (red above, green below the median value, and black, samples with signal intensity to background of 2 or less).

FIG. 1C, FIG. 1D: Western blot analysis of p53, MDM2, phosphor(p)-MDM2, c-MYC, p21 and Gapdh (FIG. 1C) and time course of CDKN1A mRNA expression by RT-PCR in Nutlin-3a treated (10 μM) MM1s cells (FIG. 1D). The PCR products were normalized to ACTIN expression. Values represent mean observed in 4 different studies ±SD.

FIG. 1E: Kinetics of miR-194, miR-192, miR-215 and miR-34a in MM1s cells after Nutli-3a treatment, measured by qRT-PCR and Northern blot analysis. Lines represent relative fold-changes between DMSO and Nutlin-3a treatment ±SD. RNU44 (qRT-PCR) and RNU6B (Northern blot) expression was used for normalization.

FIG. 1F, FIG. 1G, FIG. 1H: miR-192 (FIG. 1F), miR-215 (FIG. 1G) and miR-194 (FIG. 1H) relative expression in CD138+ PCs from healthy, MGUS and MM samples with determined by Taqman q-RT PCR assay. Each data sample was normalized to the endogenous reference RNU44 and RNU48 by use of the 2-ct method. The relative expression values were used to design box and whisker plots. Dots in the boxes indicate outlier points. Kruskal-Wallis analysis assessed that the 3 miRNAs were differentially expressed among MGUS samples versus MM PCs samples of the Bartlett test P value d.001.

FIGS. 2A-2D: miR-194-2-192 cluster is induced following p53 activation:

FIG. 2A: Luciferase reporter activity of promoter constructs of miR-192-194-2 cluster on chromosome 11q13.1 in MM1s cells after p53 transfection. The arrow above construct P1 indicates the position of the transcription start site +1. p53 binding sites (BD) are indicated (blue box).

FIG. 2B: Relative luciferase activity of P7 reporter construct. The magnified sequence highlighted in blue shows the location of the El Deiry p53 consensus binding sites in P7 construct sequence [SEQ ID NO:55]. Deletions introduced into the P7 construct are shown in yellow (X) showing abolition of the promoter activity.

FIG. 2C: Chip assay after 24 hr of p53 non genotoxic activation, showing binding of p53 to the miR-192-194-2 cluster promoter in vivo in MM1s cells.

FIG. 2D: Luciferase activity of empty vector (EV), P2 and P10 reporter constructs after non genotoxic activation of p53 and MDM-2 mRNA silencing. Luciferase activities were normalized by Renilla luciferase activities. Values represent mean±SD from three experiments.

FIGS. 3A-3J: miR-192, miR-194 and miR-215 induce decrease of proliferation and cell cycle arrest in WT TP53 MM cells:

FIG. 3A, FIG. 3B, FIG. 3C, FIG. 3D: MTS assay performed in MM1s (FIG. 3A), NCI-929 (FIG. 3B), KMS28BM (FIG. 3C) and RPMI-8226 (FIG. 3D) cell lines. Cells were transfected with miR-192, miR-215 and scrambled sequence (Scr) and were harvested at 24, 48, and 72 hrs after transfection. P values are indicated.

FIG. 3E, FIG. 3F: Soft agar colony suppression assay in WT TP53 and mutant TP53 MM cell lines after miRNAs transduction by lentivectors.

FIG. 3G, FIG. 3H, FIG. 31, FIG. 3J: Flow cytometry analysis in MM1s (FIG. 3G), NCI-H929 (FIG. 3H) and KMS28BM (FIG. 31) cells (miR-192, miR-194, miR-215 and Scr transfected) at 48 hr of transfection, after first being arrested and synchronized in G2/M phase by Nocodazole for 16 hr. Apoptosis in KMS28BM was evaluated by caspase-3 activity (FIG. 3J). All experiments were performed in triplicate ±SD.

FIGS. 4A-4F: miRNA-192, miR-194 and miR-215 effect on MDM2 protein and mRNA levels:

FIG. 4A: MM1s and NCIH929 cells (pre-miRNA-192, pre-miRNA-194, pre-miRNA-215, Scr sequence-transfected) were harvested at 72 hr after transfection and 12 hr Nutlin-3a treatment (10 μM). Whole cell lysates were subjected to Western blotting using p53, MDM2, p21, and Gapdh antibodies. Densitometric analysis showing the effect of miR-194 (white bars), miR-192 (grey bars), miR-215 (black bars) compared to Scr sequence (green bars) transfected cells of endogenous p53, MDM2 and p21 in MM1s (FIG. 4B) and NCI-H929 (FIG. 4C) Nutlin-3a treated.

FIG. 4D: Immununoblot analysis showing p53, MDM2 and p21 protein expression after 48 hr of miR192, miR-194, miR-215 (pool) and Scr ASOs transfection in MM1s and NCI-H929 cells after 12 hr of treatment with 10 μM Nutlin-3a.

FIG. 4E: Gapdh was internal loading control and densitometric analysis was reported.

FIG. 4F: MDM2 mRNA expression normalized for GAPDH mRNA expression in MM1s and NCIH929 cells miRNAs or Scr transfected after Nutlin-3a treatment (6-12 hr).

FIG. 4G: miRNAs predicted to interact with HDM2 gene in several consensus binding sites (x) at its 3′-UTR, according to “in silico” RNA-22 prediction software. Luciferase assay showing decreased luciferase activity in cells co-transfected with pGL3-MDM2-3′UTR and miR-192, miR-194, miR-215 and Scr sequence. See also FIG. 18. All experiments were performed in triplicate ±SD.

FIG. 4H: MDM2 mRNA relative expression in CD138+ PCs from healthy, MGUS and MM samples with determined by RT-PCR. Each data sample was normalized to the endogenous reference ACTIN by use of the 2-ct method. Kruskal-Wallis analysis assessed that MDM2 mRNA is differentially expressed among the healthy and MGUS samples vs MM PCs samples of the Bartlett test P value (<0.01).

FIG. 41: Graphic of the negative Spearman correlation coefficient (p=−0.698) corresponding to a decreasing monotonic trend between log of MDM2 mRNA relative expression and log of miR-192 relative expression (p<0.001, N=33).

FIGS. 5A-5E: miR-192, miR-194 and miR-215 increase sensitivity to MI-219 in vitro and in vivo by targeting MDM2:

FIG. 5A: Effects of miR-192, miR-194 and miR-215 on endogenous p53, p21 and MDM2 levels (Western blots) in MM1s cells treated with MI-219 at different concentrations.

FIG. 5B: Densitometric analysis only for p53 in untreated cells and for p53 and MDM2 protein levels in 2.5, 5 and 10 μM MI-219-treated cells. All experiments were performed in triplicate ±SD.

FIG. 5C: Apoptotic effect at different concentrations and time points for each miRNA transfected cells was assessed by caspase-3 activation assay.

FIG. 5D: Apoptosis associated with the pool of these miRNAs upon MI-219 treatment (24 h) at different concentration (2.5-10 μM) was evaluated by Annexin V. All experiments were performed in triplicate ±SD.

FIG. 5E: Gfp/Luc+MM1s cells were injected subcutaneously into the flanks of nude mice; at 3 wk post-injection, mice with comparable tumor sizes were selected for treatment (untreated). In vivo confocal imaging of GFP+/Luc+ MM cells engrafted in athymic nu/nu mice after 2 wk of combined treatment with oral MI-219 or Vehicle (VE) plus pre-microRNA pool or Scr sequence directly into the tumors.

FIGS. 6A-6H: miR-192 and miR-215 regulate IGF-1 and IGF1-R expression in MM cells:

FIG. 6A, FIG. 6B: Western blot showing IGF-1R and IGF-1 expression after miR-192 and miR-215 transfection using pre (FIG. 6A) and ASOs (FIG. 6B) for miR-192, miR-215, miR-194 and Scr in MM1s cells treated for 12 hr with Nutlin-3a.

FIG. 6C: Western blots after IGF-1 knockdown in MM1s (si-RNA) using anti-IGF-1R, IGF-1 and Gapdh antibodies.

FIG. 6D, FIG. 6E: miRNAs predicted to interact with IGF-1 and IGF-1R gene at their 3′-UTR, according to “in silico” Target Scan (IGF-1) and RNA-22 (IGF-1R) prediction software (see also FIG. 20). Luciferase assay showing decreased luciferase activity in MM1s cells co-transfected with pGL3-IGF-1-3′UTR [SEQ ID NO:57] (FIG. 6D); or pGL3-IGF1R-3′UTR [SEQ ID NOs: 59 and 60], respectively in order of appearance] (full) (FIG. 6E) and miR-192 [SEQ ID NO:56], miR-194, miR-215 [SEQ ID NO:58] or Scr. Deletion of 6 bases in all putative consensus sequences on IGF-1-3′-UTR abrogates these effect (Del) (FIG. 6D). See also FIG. 20. Bars indicate relative luciferase activity ±SD. All experiments were performed in triplicate.

FIG. 6F, FIG. 6G, FIG. 6H: Immunofluorescence using anti-IGF-1R (FIG. 6F) and anti-IGF-1 (FIG. 6G) in red and blue nuclear DNA, from CD138+ PCs from 9 MM subjects transfected with miR-192 and miR-215 (pool) or Scr and intensity of the signal was assessed ±SD. Original magnification for all images was ×400. The efficiency of the transfection in the 9 samples was evaluated using fluorescent double strand RNA oligos (FIG. 6H).

FIGS. 7A-7E: miR-194, miR-215 and miR-194 block invasion ability of MM cells:

FIG. 7A: MM1s and RPMI-8226 cells (pre-miRNA-192, 194, 215, Scr-transfected) were harvested 72 hr after transfection. Whole cell lysates were immunoblotted using IGF-1, IGF-1R, pS6, S6, p-Akt, Akt and Gapdh antibodies; Scr sequence and miR-194 transfected cells served as controls. The experiments were performed in triplicate.

FIG. 7B: Intra-epithelial migration assay in MM cells miRNAs transfected using HS-5 cells at different concentrations of IGF-1 as attractant. Bars indicate relative fold change of migration compared with the control ±SD.

FIG. 7C: In vivo confocal imaging. 8×10⁶GFP+/Luc+ MM1s cells were transfected using either pre-miRNA-192, miR-194, miR-215 and Scr RNA oligos and then iv injected into mice immediately after transfection. After 1 wk the mice were miRNAs iv injected (10 ug) once a wk for 4 wk and the bioluminescence intensity was assessed before every injection.

FIG. 7D: Representative bioluminescence imaging (BLD after 5 wk from the injection.

FIG. 7E: Bone marrow cells from the mice used for the experiment were isolated and human CD-138 positive cells (engrafted cells) were detected using anti-CD-138 antibody by flow cytometry (P2 fraction).

FIG. 8: miR-192, miR-215 and miR-194 impair the p53/MDM2 auto-regulatory loop. Model to illustrate the possible role of miR-192, miR-194 and miR-215 in control of MDM2 and IGF-1/IGF-1R pathways in MM cells.

FIG. 9: Table 1. miRNAs differentially expressed between WT TP53 versus Mutant TP53. MM cell lines.

FIG. 10: Table 2. miRNAs differentially expressed between MM1s cells Nutlin-3a treated versus MM1s cells DMSO treated

FIGS. 11A-11B: p53 and MDM2 expression in MM cell lines used for microarray experiments:

FIG. 11A: 80 μg of whole cell lysate of MM cell lines used for microarray experiments, were subjected to Western blot analysis using p53, MDM2 and Gadph antibodies.

FIG. 11B: MDM2 mRNA levels in MM cell lines carrying WT TP53 compared to normal PCs (N=4). Relative expression of MDM2 mRNA in MM1s, NCI-H929 and KMS28BM cell lines compared to the average MDM2 mRNA expression of 4 normal PCs. The PCR products for the MDM2 gene were normalized to GAPDH mRNA expression.

FIG. 12A-12C: miR-34a, miR-194 and miR-192 expression are related to TP53 status in MM cells:

FIG. 12A: miR-34a, miR-194 and miR-192 relative expression in WT TP53 (MM1s, NCI-H929, KMS28BM) and Mutant/Null TP53 cells (RPMI-8226; U266, JJN3) measured by q-RT-PCR. Bars represent relative fold changes, expressed in 2̂-(ΔCT) values ±SD obtained from three independent experiments. RNU44 expression was used for normalization.

FIG. 12B: Kinetics of activation of miR-15a, miR-29a and miR-29b in MM1s cells upon Nutlin-3a treatments, measured by qRT-PCR and Northern blot analysis. Lines represent relative fold changes, expressed in 2̂(ΔCT) values ±SD obtained from three independent experiments. RNU44 expression was used for normalization for the qRT-PCR experiments and RNU6 for the northern blot analysis.

FIG. 12C: Time course of MYC mRNA expression in Nutilin-3a treated MM1s cells by RT-PCR. The PCR product was normalized to ACTIN mRNA expression. Values represent mean±SD from three experiments. The kinetics of miR-29a, miR-29b and miR-15a looks related more to c-MYC repression than p53 activation.

FIG. 13A-13F: miR-192, miR-194 and miR-215 re-expression is dependent on p53 activation:

FIG. 13A, FIG. 13C, FIG. 13E: Western analysis for p53, MDM2 and Gapdh in NCI-H929 (WT TP53) (FIG. 13A), RPMI-8226 (Mut TP53) (FIG. 13C) and U266 (Mut TP53) (FIG. 13E) cell lines after different times of Nutlin-3a treatment. All experiments were performed in triplicate.

FIG. 13B, FIG. 13D, FIG. 13F: Stem loop q-RT-PCR showing the time course of miR-192, miR-194, miR-215 and miR-34a expression in NCI-H929 (FIG. 13B) RPMI-8226 (FIG. 13D) and U266 (FIG. 13F) cells Nutlin-3a treated compared to DMSO treatment. The PCR products were normalized to RNU6B expression. Bar-graphs represent mean values observed in four separate studies ±SD.

FIGS. 14A-14D: miR-192, miR-194 and miR-215 are re-expressed in primary MM PCs upon Nutlin-3a treatment:

FIG. 14A: Representative fax analysis of purified CD-138+ plasma cells with purity more than 90% using passive selection method (Stem-Cell) from primary samples that the inventors used for our experiments ±SD (33 MM and 14 MGUS subjects). MM1s cells were used as positive control and the non selected cells as the negative control.

FIG. 14B: Western analysis showing p53 and MDM2 expression after Nutlin-3a overnight treatment in 3 different subjects, 2 with TP53 deletion (Pt-1 and Pt-2) and 1 with WT (Normal) TP53 (Pt3).

FIG. 14C: CDKN1A mRNA expression by RT-PCR in CD-138+ PCs obtained from 8 different subjects after 12 h of Nutlin-3a treatment. The PCR product was normalized to ACTIN mRNA expression. The bar-graph represents the mean values observed in four separate studies ±SE.

FIG. 14D: miR-194, miR-192, miR-215 and miR-34a expression in primary tumor samples, after Nutlin-3a treatment, measured by stem loop qRT-PCR. Lines represent relative fold-changes between DMSO and Nutlin-3a treatment. Stem loop q-RT-PCR values were normalized to RNU44 expression. The bar graphs in FIG. 14C and FIG. 14D are representative of the 8 samples used for primary culture and Nutlin-3a treatments.

FIG. 15: p53 interacts with p53 consensus sequence up-stream of miR-194-1-215 cluster on chromosome 1(q41) in MM cells. Chip assay with anti-p53 or normal IgG from the same animal after 24 hr of p53 non genotoxic activation, revealed binding of p53 to the miR-194-1-215 cluster promoter in vivo in MM1s cells. ChIP primers were designed to amplify the region containing the putative p53 binding site in the pri-miR-194-1-215 promoter (−2.7 kb from the cluster). p53-responsive CDKN1A gene promoter associated with p53 was used as positive control, whereas amplification of a MT-RNR2 gene portion yielded very little background signals and served as negative control.

FIG. 16A-16D: miR-192, miR-194 and miR-215 regulate CDKN1A and MDM2 mRNA level in MM cells:

FIG. 16A, FIG. 16B, FIG. 16C: MM1s, NCI-H929, KMS28BM and RPMI-8226 cells (pre-miRNA-192, 194, 215, Scr sequence-transfected) were harvested at 48 hr after transfection and CDKN1A (FIG. 16A), TP53 (FIG. 16B) and MDM2 (FIG. 16C) mRNA expression level was assessed. The PCR products for the genes were normalized to ACTIN mRNA expression. The bar-graphs represent mean values observed in four separate studies ±SD.

FIG. 16D: miRNA-192-194 and 215 effects on MDM2 protein level in Mut TP53 cells (RPMI-8226). RPMI-8226 cells (miR-192, miR-194, miR-215, Scr sequence-transfected) were harvested at 72 h after transfection. Whole cell lysates were subjected to Western blot using MDM2 and Gapdh antibodies. Bars indicate MDM2 protein relative fold change ±SD. Gapdh was internal loading control and used for the densitometry analysis. The experiment was performed in triplicate.

FIGS. 17A, FIG. 17B: Assessment of expression of miRNAs in MM transfected cells using pre-miR-192, -194 and -215 (FIG. 17A), and anti-sense oligo-nucleotides (ASOs) (FIG. 17B). MM1s and NCI-H929 cells transfected with pre-miRNAs or ASOs were harvested at 72 hr after transfection and the level of the microRNAs was assessed by stem loop q-RT-PCR for each miRNA compared to the Scr sequence-transfected cells. Bar-graphs represent the mean values observed in four separate studies ±SD.

FIGS. 18A-18I: miR-192, miR-194 and miR-215 target human MDM2 (HDM2):

FIG. 18A: Representation of the full length human MDM2 mRNA (HDM2).

FIG. 18B, FIG. 18C, FIG. 18D, FIG. 18E: miRNAs predicted to interact with HDM2 mRNA at several consensus binding sites in its 3′-UTR, according to “in silico” RNA-22 prediction software with a folding energy >−27 Kcal/mol. The MREs are indicated by the triangles. FIGS. 18B-18E disclose SEQ ID NOs:61-68, respectively, in order of appearance.

FIG. 18F, FIG. 18G, FIG. 18H, FIG. 18I: Luciferase assay showing decreased luciferase activity in cells co-transfected with pGL3MDM2-3′UTR containing the specific binding sites (˜1 kb) for each miRNA. Specifically: CS2117 and CS5974 constructs for miR-194 (FIG. 18F-FIG. 18H) and CS3975 and CS6360 constructs for miR-192 and 215 (FIG. 18G-FIG. 18I). Deletion of six bases in all putative consensus sequences abrogates this effect (Del). Bars indicate firefly luciferase activity normalized to Renilla luciferase activity ±SD.

FIGS. 19A-19D: Effect of Nutlin-3a treatment on IGF-R and IGF-1 protein expression in MM cells with different TP53 status. WT TP53 (MM1s and NCI-H929) (FIG. 19A, FIG. 19B) and Mutant TP53 (RPMI-8226 and U266) (FIG. 19C, FIG. 19D) cells were treated with Nutlin-3a (10 μM) or DMSO vehicle and whole cell lysates collected at different time points were immunoblotted using antisera against IGF-1R, IGF-1, p53, MDM2. Gapdh was used as loading control. A decrease in IGF-R and IGF-1 protein level is shown only in TP53 WT cells upon Nutlin-3a treatment.

FIGS. 20A-20D. miR-192 and 215 target IGF-1R:

FIG. 20A: Representation of the full length IGF-1R mRNA.

FIG. 20B, FIG. 20C: miRNAs predicted to interact with IGF-1R gene in several consensus binding sites at its 3T-UTR, according to “in silico” RNA-22 prediction software with a folding energy >−27 Kcal/mol. FIGS. 20B-20C disclose SEQ ID NOs:69-72, respectively, in order of appearance.

FIG. 20D, FIG. 20E: Luciferase assay showing decreased luciferase activity in cells co-transfected with 2 different constructs (1 kb each) of pGL3-IGF-1R-3TUTR and miR-215 (FIG. 20D-FIG. 20E) and miR-192 (FIG. 20E) but not with miR-194 and Scr sequence (FIG. 20D-FIG. 20E). Deletion of six bases in all putative consensus sequences abrogates this effect (Del) (FIG. 20D-FIG. 20E). Bars indicate firefly luciferase activity normalized to Renilla luciferase activity ±SD.

FIG. 21A-21C: miR-192 and miR-215 affect the ability of MM cells to adhere and migrate in response to IGF-1:

FIG. 21A, FIG. 21B: MM1s and RPMI-8226 cells (pre-miRNA-192, -194, -215, Scr sequence-transfected) at 48 hr after transfection were harvested, treated with calcein and incubated with IGF-1 (50 ng/ml) and their ability to adhere to fibronectin plates was assessed by fluorescence assay. MM1s (FIG. 21A) and RPMI-8226 (FIG. 21B) with ectopic re-expression of miR-192, 215 and also miR-194 lost their ability to respond to IGF-1 treatment compare to Scr sequence.

FIG. 21C: Intra-epithelial migration assay in MM1s and RPMI-8226 cells (pre-miRNA-192, -194, -215, Scr-transfected) using HS-27A stromal cell as cellular layer at different concentrations of IGF-1 as attractant. Bars indicate relative fold change of migration compared with the control. All experiments were performed in triplicate.

FIGS. 22A-22D: The promoter region of miR-194-2&192 is methylated in MM cell lines:

FIG. 22A: Representation of the genomic region of miR-194-2&192 obtained from University of California Santa Cruz genome browser (2006). The red arrow is the region analyzed for the methylation study, including the p53 consensus sequence.

FIG. 22B: Combined bisulfate restriction analysis (COBRA) in 9 MM cell lines. Universal methylated DNA from Millipore was used as positive control and normal CD-138+ plasma cells as negative control. The digestion of PCR products coming from methylated DNA was carried out with TaqI for the region R.

FIG. 22C: Stem-loop q-RT-PCR for miR-192 and miR-194 and RT-PCR for SOCS-1 genes normalized to RN44 and ACTIN respectively, expressed as fold increases after 3 days of treatment with 5-Azacitidine (10 μM) compared to DMSO treated cells. Bars indicate relative fold change of migration compared with control. All experiments were performed in triplicate.

FIG. 22D: Illustration of the p53—miR-192,194,215—MDM2 auto regulatory loops, showing the central role played by the miRs in determining the balance of p53 suppressor and the MDM2 oncoprotein expression levels.

FIG. 23: Table 3. Clinical data for subject samples.

FIG. 24: Structures of Nutlin 3a and MI-219.

### DETAILED DESCRIPTION OF THE PREFERRED EMBODIMENT

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

The present invention provides research tools, diagnostic methods, and therapeutical methods and compositions using the knowledge derived from this discovery. The invention is industrially applicable for the purpose of sensitizing tumor cells to drug-inducing apoptosis and also to inhibit tumor cell survival, proliferation and invasive capabilities.

### ABBREVIATIONS

DNA Deoxyribonucleic acid
EV Empty vector
HMD2 Human double minute 2
IGF Insulin growth factor
ISH In situ hybridization
miR MicroRNA
miRNA MicroRNA
mRNA Messenger RNA
MM Multiple Myeloma
MGUS Monoclonal gammopathy of undetermined significance
MDM2 Murine double minute 2
p53 p53 tumor suppressor
PCR Polymerase chain reaction
pre-miRNA Precursor microRNA
qRT-PCR Quantitative reverse transcriptase polymerase chain reaction
RNA Ribonucleic acid
siRNA Small interfering RNA
snRNA Small nuclear RNA
SVM Support vector machines
WT Wild type/s

### TERMS

It is to be understood that both the foregoing general description and the following detailed description are exemplary and explanatory only and are not intended to limit the scope of the current teachings. In this application, the use of the singular includes the plural unless specifically stated otherwise.

The use of the word “a” or “an” when used in conjunction with the term “comprising” in the claims and/or the specification may mean “one,” but it is also consistent with the meaning of “one or more,” “at least one,” and “one or more than one.”

Also, the use of “comprise”, “contain”, and “include”, or modifications of those root words, for example but not limited to, “comprises”, “contained”, and “including”, are not intended to be limiting. The term “and/or” means that the terms before and after can be taken together or separately. For illustration purposes, but not as a limitation, “X and/or Y” can mean “X” or “Y” or “X and Y”.

It is understood that a miRNA is derived from genomic sequences or a gene. In this respect, the term “gene” is used for simplicity to refer to the genomic sequence encoding the precursor miRNA for a given miRNA. However, embodiments of the invention may involve genomic sequences of a miRNA that are involved in its expression, such as a promoter or other regulatory sequences.

The term “miRNA” generally refers to a single-stranded molecule, but in specific embodiments, molecules implemented in the invention will also encompass a region or an additional strand that is partially (between 10 and 50% complementary across length of strand), substantially (greater than 50% but less than 100% complementary across length of strand) or fully complementary to another region of the same single-stranded molecule or to another nucleic acid. Thus, nucleic acids may encompass a molecule that comprises one or more complementary or self-complementary strand(s) or “complement(s)” of a particular sequence comprising a molecule. For example, precursor miRNA may have a self-complementary region, which is up to 100% complementary miRNA probes of the invention can be or be at least 60, 65, 70, 75, 80, 85, 90, 95, or 100% complementary to their target.

The term “combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB.

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Anticancer agent and anticancer drug: Any therapeutic agents (e.g., chemotherapeutic compounds and/or molecular therapeutic compounds), antisense therapies, radiation therapies, or surgical interventions, used in the treatment of hyperproliferative diseases such as cancer (e.g., in mammals).

Adjunctive therapy: A treatment used in combination with a primary treatment to improve the effects of the primary treatment. For example, a subject diagnosed with HCC may undergo liver resection as a primary treatment and antisense miR-221 and miR-222 therapy as an adjunctive therapy.

Candidate: As used herein, a “candidate” for therapy is a subject that has multiple myeloma (MM).

Clinical outcome: Refers to the health status of a subject following treatment for a disease or disorder; or in the absence of treatment. Clinical outcomes include, but are not limited to, an increase in the length of time until death, a decrease in the length of time until death, an increase in the chance of survival, an increase in the risk of death, survival, disease-free survival, chronic disease, metastasis, advanced or aggressive disease, disease recurrence, death, and favorable or poor response to therapy.

Control: A “control” refers to a sample or standard used for comparison with an experimental sample, such as a tumor sample obtained from a subject. In some embodiments, the control is a sample obtained from a healthy subject or a non-cancerous sample obtained from a subject diagnosed. In some embodiments, the control is a historical control or standard value (i.e., a previously tested control sample or group of samples that represent baseline or normal values, such as the level in a non-cancerous sample).

Cytokines: Proteins produced by a wide variety of hematopoietic and non-hematopoietic cells that affect the behavior of other cells. Cytokines are important for both the innate and adaptive immune responses.

Decrease in survival: As used herein, “decrease in survival” refers to a decrease in the length of time before death of a subject, or an increase in the risk of death for the subject.

Detecting level of expression: For example, “detecting the level of miR-192 expression” refers to quantifying the amount of miR-192 present in a sample. Detecting expression of miR-192, or any microRNA, can be achieved using any method known in the art or described herein, such as by qRT-PCR. Detecting expression of miR-192 includes detecting expression of either a mature form of miR-192 or a precursor form that is correlated with miR-192 expression. Typically, miRNA detection methods involve sequence specific detection, such as by RT-PCR. miR-specific primers and probes can be designed using the precursor and mature miR nucleic acid sequences, which are known in the art and include modifications which do not change the function of the sequences.

Functional p53: Wild-type p53 expressed at normal, high, or low levels and mutant p53 that retains at least 5% of the activity of wild-type p53, e.g., at least 10%, 20%, 30%, 40%, 50%, or more of wild-type activity.

p53-related protein: Proteins that have at least 25% sequence homology with p53, have tumor suppressor activity, and are inhibited by interaction with MDM2 or MDM2-related proteins. Examples of p53-related proteins include, but are not limited to, p63 and p73.

MDM2-related protein: Proteins that have at least 25% sequence homology with MDM2, and interact with and inhibit p53 or p53-related proteins. Examples of MDM2-related proteins include, but are not limited to, MDMX and HDM2.

MicroRNA (miRNA, miR): Single-stranded RNA molecules that regulate gene expression. MicroRNAs are generally 21-23 nucleotides in length. MicroRNAs are processed from primary transcripts known as pri-miRNA to short stem-loop structures called precursor (pre)-miRNA and finally to functional, mature microRNA. Mature microRNA molecules are partially complementary to one or more messenger RNA molecules, and their primary function is to down-regulate gene expression. MicroRNAs regulate gene expression through the RNAi pathway.

miR-expression: As used herein, “low miR-expression” and “high miR-expression” are relative terms that refer to the level of miR/s found in a sample. In some embodiments, low and high miR-expression are determined by comparison of miR/s levels in a group of non-cancerous and MM samples. Low and high expression can then be assigned to each sample based on whether the expression of a miR in a sample is above (high) or below (low) the average or median miR expression level. For individual samples, high or low miR expression can be determined by comparison of the sample to a control or reference sample known to have high or low expression, or by comparison to a standard value. Low and high miR expression can include expression of either the precursor or mature forms of miR, or both.

Normal cell: A cell that is not undergoing abnormal growth or division. Normal cells are non-cancerous and are not part of any hyperproliferative disease or disorder.

Anti-neoplastic agent: Any compound that retards the proliferation, growth, or spread of a targeted (e.g., malignant) neoplasm.

Prevent, preventing, and prevention: A decrease in the occurrence of pathological cells (e.g., hyperproliferative or neoplastic cells) in an animal. The prevention may be complete, e.g., the total absence of pathological cells in a subject. The prevention may also be partial, such that the occurrence of pathological cells in a subject is less than that which would have occurred without the present invention.

Subject: As used herein, the term “subject” includes human and non-human animals. The preferred subject for treatment is a human. “Subject” and “subject” are used interchangeably herein.

Pharmaceutically acceptable vehicles: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compounds, molecules or agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Screening: As used herein, “screening” refers to the process used to evaluate and identify candidate agents that affect MM. In some cases, screening involves contacting a candidate agent (such as an antibody, small molecule or cytokine) with cancer cells and testing the effect of the agent. Expression of a microRNA can be quantified using any one of a number of techniques known in the art and described herein, such as by microarray analysis or by qRT-PCR.

Pharmaceutically acceptable salt: Any salt (e.g., obtained by reaction with an acid or a base) of a compound of the present invention that is physiologically tolerated in the target animal (e.g., a mammal). Salts of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts. Examples of bases include, but are not limited to, alkali metal (e.g., sodium) hydroxides, alkaline earth metal (e.g., magnesium) hydroxides, ammonia, and the like. Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, mesylate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound

Therapeutically effective amount: That amount of the therapeutic agent sufficient to result in amelioration of one or more symptoms of a disorder, or prevent advancement of a disorder, or cause regression of the disorder. For example, with respect to the treatment of cancer, in one embodiment, a therapeutically effective amount will refer to the amount of a therapeutic agent that decreases the rate of tumor growth, decreases tumor mass, decreases the number of metastases, increases time to tumor progression, or increases survival time by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

Sensitize and sensitizing: Making, through the administration of a first agent, an animal or a cell within an animal more susceptible, or more responsive, to the biological effects (e.g., promotion or retardation of an aspect of cellular function including, but not limited to, cell division, cell growth, proliferation, invasion, angiogenesis, necrosis, or apoptosis) of a second agent. The sensitizing effect of a first agent on a target cell can be measured as the difference in the intended biological effect (e.g., promotion or retardation of an aspect of cellular function including, but not limited to, cell growth, proliferation, invasion, angiogenesis, or apoptosis) observed upon the administration of a second agent with and without administration of the first agent. The response of the sensitized cell can be increased by at least 10%, at least 20%, at least 30%, at least 40%, at least 50%, at least 60%, at least 70%, at least 80%, at least 90%, at least 100%, at least 150%, at least 200%, at least 350%, at least 300%, at least 350%, at least 400%, at least 450%, or at least 500% over the response in the absence of the first agent.

Small molecule: A molecule, typically with a molecular weight less than about 1000 Daltons, or in some embodiments, less than about 500 Daltons, wherein the molecule is capable of modulating, to some measurable extent, an activity of a target molecule.

Therapeutic: A generic term that includes both diagnosis and treatment.

Therapeutic agent: A chemical compound, small molecule, or other composition, such as an antisense compound, antibody, protease inhibitor, hormone, chemokine or cytokine, capable of inducing a desired therapeutic or prophylactic effect when properly administered to a subject. For example, therapeutic agents include agents that prevent or inhibit development or metastasis. As used herein, a “candidate agent” is a compound selected for screening to determine if it can function as a therapeutic agent. “Incubating” includes a sufficient amount of time for an agent to interact with a cell or tissue. “Contacting” includes incubating an agent in solid or in liquid form with a cell or tissue. “Treating” a cell or tissue with an agent includes contacting or incubating the agent with the cell or tissue.

Therapeutically effective amount: A quantity of a specified pharmaceutical or therapeutic agent sufficient to achieve a desired effect in a subject, or in a cell, being treated with the agent. For example, this can be the amount of a therapeutic agent that alters the expression of miR/s, and thereby prevents, treats or ameliorates the disease or disorder in a subject. The effective amount of the agent will be dependent on several factors, including, but not limited to the subject or cells being treated, and the manner of administration of the therapeutic composition.

In some embodiments, the control is non-cancerous cell/tissue sample obtained from the same subject. In other embodiments, the control is a sample obtained from a healthy subject, such as a donor. In another example, the control is a standard calculated from historical values. Cancerous samples and non-cancerous tissue samples can be obtained according to any method known in the art.

In some embodiments, screening comprises contacting the candidate agents with cells. The cells can be primary cells obtained from a subject, or the cells can be immortalized or transformed cells.

The candidate agents can be any type of agent, such as a protein, peptide, small molecule, antibody or nucleic acid. In some embodiments, the candidate agent is a cytokine. In some embodiments, the candidate agent is a small molecule. Screening includes both high-throughout screening and screening individual or small groups of candidate agents.

Methods of Detecting RNA Expression

The sequences of precursor microRNAs (pre-miRNAs) and mature miRNAs are publicly available, such as through the miRBase database, available online by the Sanger Institute (see Griffiths-Jones et al., Nucleic Acids Res. 36:D154-D158, 2008; Griffiths-Jones et al., Nucleic Acids Res. 34:D140-D144, 2006; and Griffiths-Jones, Nucleic Acids Res. 32: D109-D111, 2004).

Detection and quantification of RNA expression can be achieved by any one of a number of methods well known in the art (see, for example, U.S. Patent Application Publication Nos. 2006/0211000 and 2007/0299030, herein incorporated by reference) and described below. Using the known sequences for RNA family members, specific probes and primers can be designed for use in the detection methods described below as appropriate.

In some cases, the RNA detection method requires isolation of nucleic acid from a sample, such as a cell or tissue sample. Nucleic acids, including RNA and specifically miRNA, can be isolated using any suitable technique known in the art. For example, phenol-based extraction is a common method for isolation of RNA. Phenol-based reagents contain a combination of denaturants and RNase inhibitors for cell and tissue disruption and subsequent separation of RNA from contaminants. Phenol-based isolation procedures can recover RNA species in the 10-200-nucleotide range (e.g., precursor and mature miRNAs, 5S and 5.8S ribosomal RNA (rRNA), and U1 small nuclear RNA (snRNA)). In addition, extraction procedures such as those using TRIZOL™ or TRI REAGENT™, will purify all RNAs, large and small, and are efficient methods for isolating total RNA from biological samples that contain miRNAs and small interfering RNAs (siRNAs).

### Microarray

A microarray is a microscopic, ordered array of nucleic acids, proteins, small molecules, cells or other substances that enables parallel analysis of complex biochemical samples. A DNA microarray consists of different nucleic acid probes, known as capture probes that are chemically attached to a solid substrate, which can be a microchip, a glass slide or a microsphere-sized bead. Microarrays can be used, for example, to measure the expression levels of large numbers of messenger RNAs (mRNAs) and/or miRNAs simultaneously.

Microarrays can be fabricated using a variety of technologies, including printing with fine-pointed pins onto glass slides, photolithography using pre-made masks, photolithography using dynamic micromirror devices, ink-jet printing, or electrochemistry on microelectrode arrays.

Microarray analysis of miRNAs, for example (although these procedures can be used in modified form for any RNA analysis) can be accomplished according to any method known in the art (see, for example, PCT Publication No. WO 2008/054828; Ye et al., Nat. Med. 9(4):416-423, 2003; Calin et al., N. Engl. J. Med. 353(17):1793-1801, 2005, each of which is herein incorporated by reference). In one example, RNA is extracted from a cell or tissue sample, the small RNAs (18-26-nucleotide RNAs) are size-selected from total RNA using denaturing polyacrylamide gel electrophoresis. Oligonucleotide linkers are attached to the 5′ and 3′ ends of the small RNAs and the resulting ligation products are used as templates for an RT-PCR reaction with 10 cycles of amplification. The sense strand PCR primer has a fluorophore attached to its 5′ end, thereby fluorescently labeling the sense strand of the PCR product. The PCR product is denatured and then hybridized to the microarray. A PCR product, referred to as the target nucleic acid that is complementary to the corresponding miRNA capture probe sequence on the array will hybridize, via base pairing, to the spot at which the capture probes are affixed. The spot will then fluoresce when excited using a microarray laser scanner. The fluorescence intensity of each spot is then evaluated in terms of the number of copies of a particular miRNA, using a number of positive and negative controls and array data normalization methods, which will result in assessment of the level of expression of a particular miRNA.

In an alternative method, total RNA containing the small RNA fraction (including the miRNA) extracted from a cell or tissue sample is used directly without size-selection of small RNAs, and 3′ end labeled using T4 RNA ligase and either a fluorescently-labeled short RNA linker. The RNA samples are labeled by incubation at 30° C. for 2 hours followed by heat inactivation of the T4 RNA ligase at 80° C. for 5 minutes. The fluorophore-labeled miRNAs complementary to the corresponding miRNA capture probe sequences on the array will hybridize, via base pairing, to the spot at which the capture probes are affixed. The microarray scanning and data processing is carried out as described above.

There are several types of microarrays than be employed, including spotted oligonucleotide microarrays, pre-fabricated oligonucleotide microarrays and spotted long oligonucleotide arrays. In spotted oligonucleotide microarrays, the capture probes are oligonucleotides complementary to miRNA sequences. This type of array is typically hybridized with amplified PCR products of size-selected small RNAs from two samples to be compared (such as non-cancerous tissue and HCC liver tissue) that are labeled with two different fluorophores. Alternatively, total RNA containing the small RNA fraction (including the miRNAs) is extracted from the two samples and used directly without size-selection of small RNAs, and 3′ end labeled using T4 RNA ligase and short RNA linkers labeled with two different fluorophores. The samples can be mixed and hybridized to one single microarray that is then scanned, allowing the visualization of up-regulated and down-regulated miRNA genes in one assay.

In pre-fabricated oligonucleotide microarrays or single-channel microarrays, the probes are designed to match the sequences of known or predicted miRNAs. There are commercially available designs that cover complete genomes (for example, from Affymetrix or Agilent). These microarrays give estimations of the absolute value of gene expression and therefore the comparison of two conditions requires the use of two separate microarrays.

Spotted long Oligonucleotide Arrays are composed of 50 to 70-mer oligonucleotide capture probes, and are produced by either ink-jet or robotic printing. Short Oligonucleotide Arrays are composed of 20-25-mer oligonucleotide probes, and are produced by photolithographic synthesis (Affymetrix) or by robotic printing.

Quantitative RT-PCR

Quantitative RT-PCR (qRT-PCR) is a modification of polymerase chain reaction used to rapidly measure the quantity of a product of polymerase chain reaction. qRT-PCR is commonly used for the purpose of determining whether a genetic sequence, such as a miR, is present in a sample, and if it is present, the number of copies in the sample. Any method of PCR that can determine the expression of a nucleic acid molecule, including a miRNA, falls within the scope of the present disclosure. There are several variations of the qRT-PCR method known in the art, three of which are described below.

Methods for quantitative polymerase chain reaction include, but are not limited to, via agarose gel electrophoresis, the use of SYBR Green (a double stranded DNA dye), and the use of a fluorescent reporter probe. The latter two can be analyzed in real-time.

With agarose gel electrophoresis, the unknown sample and a known sample are prepared with a known concentration of a similarly sized section of target DNA for amplification. Both reactions are run for the same length of time in identical conditions (preferably using the same primers, or at least primers of similar annealing temperatures). Agarose gel electrophoresis is used to separate the products of the reaction from their original DNA and spare primers. The relative quantities of the known and unknown samples are measured to determine the quantity of the unknown.

The use of SYBR Green dye is more accurate than the agarose gel method, and can give results in real time. A DNA binding dye binds all newly synthesized double stranded DNA and an increase in fluorescence intensity is measured, thus allowing initial concentrations to be determined. However, SYBR Green will label all double-stranded DNA, including any unexpected PCR products as well as primer dimers, leading to potential complications and artifacts. The reaction is prepared as usual, with the addition of fluorescent double-stranded DNA dye. The reaction is run, and the levels of fluorescence are monitored (the dye only fluoresces when bound to the double-stranded DNA). With reference to a standard sample or a standard curve, the double-stranded DNA concentration in the PCR can be determined.

The fluorescent reporter probe method uses a sequence-specific nucleic acid based probe so as to only quantify the probe sequence and not all double stranded DNA. It is commonly carried out with DNA based probes with a fluorescent reporter and a quencher held in adjacent positions (so-called dual-labeled probes). The close proximity of the reporter to the quencher prevents its fluorescence; it is only on the breakdown of the probe that the fluorescence is detected. This process depends on the 5′ to 3′ exonuclease activity of the polymerase involved.

The real-time quantitative PCR reaction is prepared with the addition of the dual-labeled probe. On denaturation of the double-stranded DNA template, the probe is able to bind to its complementary sequence in the region of interest of the template DNA. When the PCR reaction mixture is heated to activate the polymerase, the polymerase starts synthesizing the complementary strand to the primed single stranded template DNA. As the polymerization continues, it reaches the probe bound to its complementary sequence, which is then hydrolyzed due to the 5′-3′ exonuclease activity of the polymerase, thereby separating the fluorescent reporter and the quencher molecules. This results in an increase in fluorescence, which is detected. During thermal cycling of the real-time PCR reaction, the increase in fluorescence, as released from the hydrolyzed dual-labeled probe in each PCR cycle is monitored, which allows accurate determination of the final, and so initial, quantities of DNA.

In Situ Hybridization

In situ hybridization (ISH) applies and extrapolates the technology of nucleic acid hybridization to the single cell level, and, in combination with the art of cytochemistry, immunocytochemistry and immunohistochemistry, permits the maintenance of morphology and the identification of cellular markers to be maintained and identified, and allows the localization of sequences to specific cells within populations, such as tissues and blood samples. ISH is a type of hybridization that uses a complementary nucleic acid to localize one or more specific nucleic acid sequences in a portion or section of tissue (in situ), or, if the tissue is small enough, in the entire tissue (whole mount ISH). RNA ISH can be used to assay expression patterns in a tissue, such as the expression of miRNAs.

Sample cells or tissues are treated to increase their permeability to allow a probe, such as a miRNA-specific probe, to enter the cells. The probe is added to the treated cells, allowed to hybridize at pertinent temperature, and excess probe is washed away. A complementary probe is labeled with a radioactive, fluorescent or antigenic tag, so that the probe's location and quantity in the tissue can be determined using autoradiography, fluorescence microscopy or immunoassay. The sample may be any sample as herein described, such as a non-cancerous or cancerous sample. Since the sequences of miR family members are known, miR probes can be designed accordingly such that the probes specifically bind the miR.

In Situ PCR

In situ PCR is the PCR based amplification of the target nucleic acid sequences prior to ISH. For detection of RNA, an intracellular reverse transcription step is introduced to generate complementary DNA from RNA templates prior to in situ PCR. This enables detection of low copy RNA sequences.

Prior to in situ PCR, cells or tissue samples are fixed and permeabilized to preserve morphology and permit access of the PCR reagents to the intracellular sequences to be amplified. PCR amplification of target sequences is next performed either in intact cells held in suspension or directly in cytocentrifuge preparations or tissue sections on glass slides. In the former approach, fixed cells suspended in the PCR reaction mixture are thermally cycled using conventional thermal cyclers. After PCR, the cells are cytocentrifuged onto glass slides with visualization of intracellular PCR products by ISH or immunohistochemistry. In situ PCR on glass slides is performed by overlaying the samples with the PCR mixture under a coverslip which is then sealed to prevent evaporation of the reaction mixture. Thermal cycling is achieved by placing the glass slides either directly on top of the heating block of a conventional or specially designed thermal cycler or by using thermal cycling ovens.

Detection of intracellular PCR products is generally achieved by one of two different techniques, indirect in situ PCR by ISH with PCR-product specific probes, or direct in situ PCR without ISH through direct detection of labeled nucleotides (such as digoxigenin-11-dUTP, fluorescein-dUTP, 3H-CTP or biotin-16-dUTP), which have been incorporated into the PCR products during thermal cycling.

Use of miR-192, miR-194 and miR-215 as predictive markers of prognosis and for identification of therapeutic agents for treatment of MM.

Thus, provided herein is a method of identifying therapeutic agents for the treatment of MM. In certain embodiments, the at least one feature of the cancer is selected from one or more of the group consisting of: presence or absence of the cancer; type of the cancer; origin of the cancer; diagnosis of cancer; prognosis of the cancer; therapy outcome prediction; therapy outcome monitoring; suitability of the cancer to treatment, such as suitability of the cancer to chemotherapy treatment and/or radiotherapy treatment; suitability of the cancer to hormone treatment; suitability of the cancer for removal by invasive surgery; suitability of the cancer to combined adjuvant therapy.

Also described herein is a method of for the determination of suitability of a cancer for treatment, wherein the at least one feature of the cancer is suitability of the cancer to treatment, such as suitability of the cancer to chemotherapy treatment and/or radiotherapy treatment; suitability of the cancer to hormone treatment; suitability of the cancer for removal by invasive surgery; suitability of the cancer to combined adjuvant therapy.

Also described herein is a method for the determination of the likely prognosis of a cancer subject comprising: i) isolating at least one tissue sample from a subject suffering from cancer; and, ii) characterizing at least one tissue sample; wherein the feature allows for the determination of the likely prognosis of the cancer subject.

The following examples are provided to illustrate certain particular features and/or embodiments. These examples should not be construed to limit the disclosure to the particular features or embodiments described.

### EXAMPLES

Tumor suppressor p53 is a transcription factor that plays a role in the regulation of cell cycle, apoptosis, DNA repair, senescence and angiogenesis. p53 is functionally impaired by mutation or deletion in nearly 50% of human cancers. In the remaining human cancers, p53 retains a wild-type (WT) status; its function, however, is inhibited by a cellular inhibitor (human double minute 2 (in humans), murine double minute 2 (in mouse). Further, HDM2/MDM2 is an essential regulator of p53 in normal cells, but its deregulated expression provides growth advantages to cells. As such, p53 is an attractive cancer therapeutic target because it can be functionally activated to eradicate cancerous cells/tumors.

MicroRNAs (miRNAs or miRs) are an abundant class of short, non protein-coding RNAs mediating posttranscriptional regulation of target genes, that have emerged as master regulators in diverse physiologic and pathologic processes and oncogenesis. microRNAs (miRNAs) are directly transactivated by p53. As shown herein, p53 and components of its pathway are targeted by certain miRNAs, thereby affecting p53 activities.

The invention is based, at least in part, on the inventor's discovery of new signal pathways in which miR-192, miR-194 and miR-215 are regulators of the MDM2/p53 auto regulatory loop, controlling the balance between p53 and MDM2 expression. Hypermethylation of the miR-194-2-192 cluster promoter in MM cell lines indicates that epigenetic down-regulation of these miRNAs (which leads to increased MDM2 mRNA and protein expression) decreases the ability of p53 to down-modulate MDM2 expression, tipping the regulatory loop in favor of MDM2. Thus, these miRNAs can be useful as important mediators in the pharmacological activation of the p53 pathway in MM cells, offering new avenues for miRNA-targeted therapies and MM treatment.

The inventors herein have now discovered the role of miRNAs in the p53 apoptotic pathway in MM cells using small-molecule inhibitors of MDM2: miR-192, miR-194 and miR-215 are mediators of the p53/MDM2 auto regulatory loop. While not wishing to be bound by theory, the inventors herein now believe that loss of expression of these miRNAs in MM contributes to p53 inactivation by sustaining expression of MDM2 and other p53 regulated proteins associated with tumor progression.

The invention is also based, at least in part, on the inventors' discoveries that: i) miR-192, miR-194 and miR-215 are silent in newly diagnosed MMs; ii) WT p53 is a transcriptional activator of miR-192, miR-194 and miR-215; iii) HDM2 mRNA is directly down-modulated by miR-192, mir-194 and miR-215; and, iv) miR-192, miR-194 and miR-215 enhance the pharmacological activity of MDM2 inhibitors.

Identification of p53-Regulated miRNAs in MM

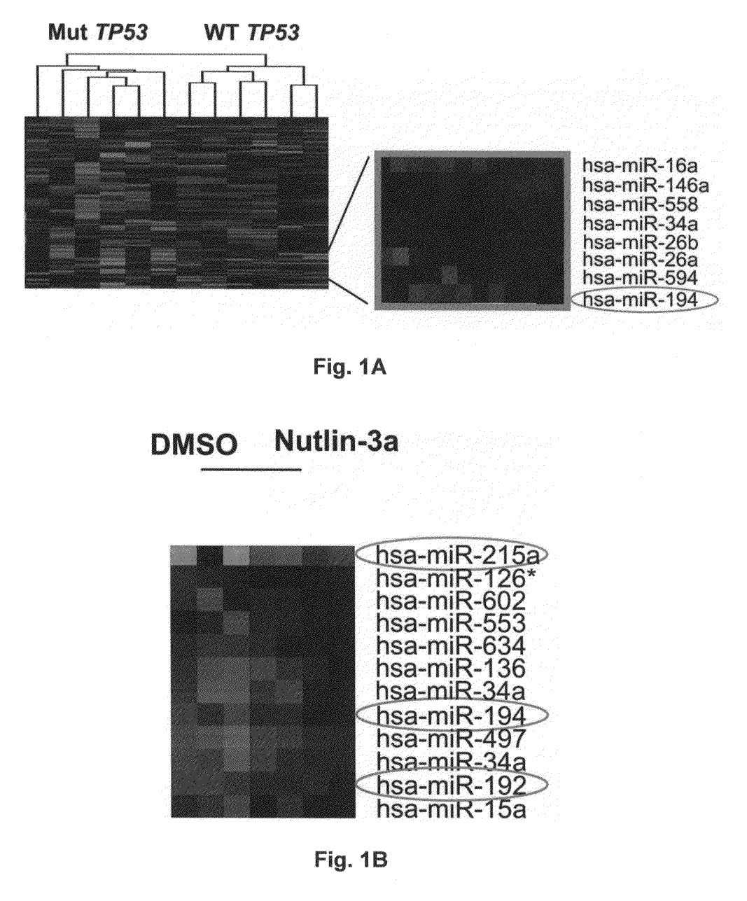

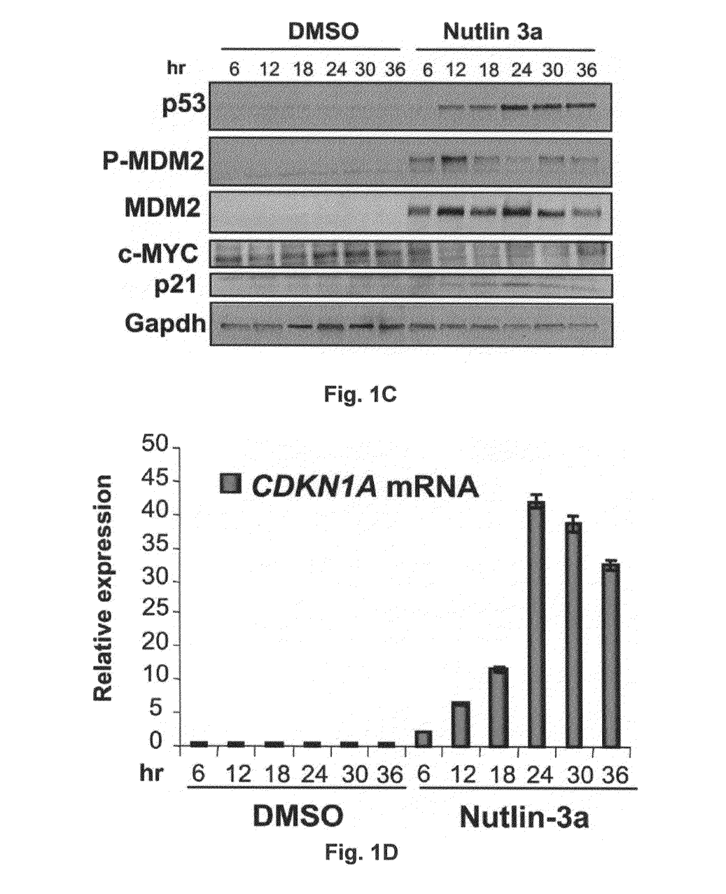

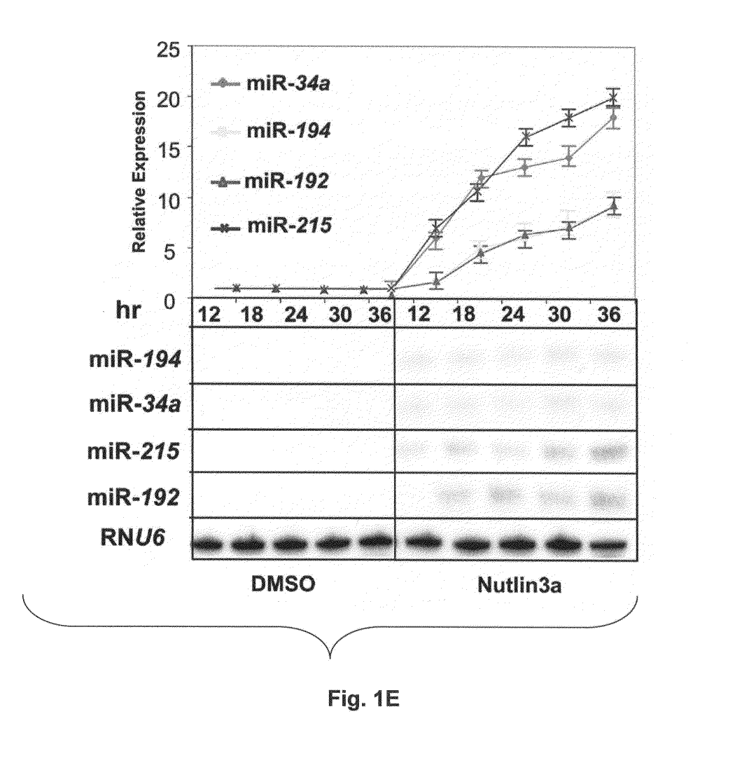

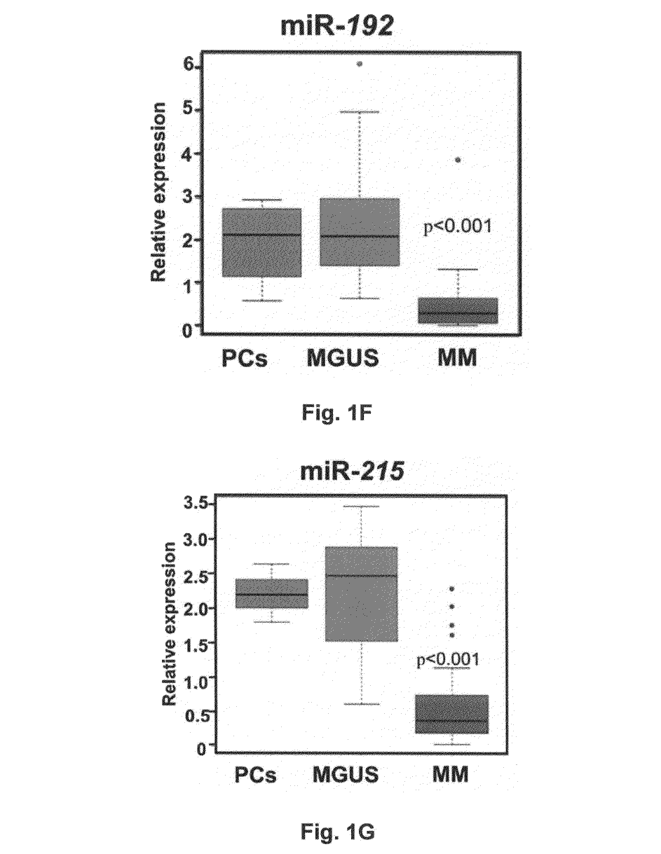

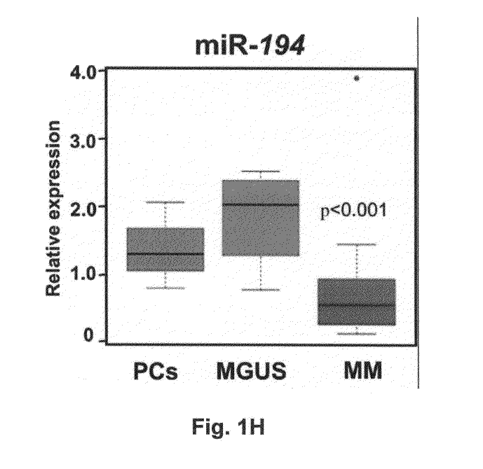

To determine whether p53 regulated miRNA pathways are functional in MM cells, the inventors performed custom microarray analysis with an expanded set of probes capable of assaying the expression of more than 500 human miRNAs. Two models for comparison of effect of p53 expression were chosen for analysis. The inventors first assessed by microarray chip analysis a specific signature associated with the presence of WT TP53 in MM cell lines as shown in FIG. 1A and FIG. 9—Table 1.

Six MM cell lines were used in the analyses: MM1s; NCI-H929; KMS28BM that retains and expresses WT TP53; RPMI-8226; U266 with mutant TP53; and, JJN3 that do not express TP53 mRNA. Western blot analysis of these cells shows p53 and MDM2 expression status (FIG. 11A) and genomic and cDNA sequence analyses confirmed the presence of WT TP53 cells in association with higher MDM2 mRNA expression (FIG. 11B).

Several differentially miRNAs expressed were identified (FIG. 9—Table 1).

The inventors performed miRNA microarray analysis after up-modulation of p53 expression in MM1s cells upon 12 hr treatment with Nutlin-3a (10 μM), a small-molecule inhibitor of MDM2 (FIG. 1B).

In response to Nutlin-3a treatment, the inventors identified expression of distinct miRNAs associated with p53 activation (FIG. 1B, FIG. 10—Table 2). Only two miRNAs were up-regulated in common in both analyses, miR-34a and miR-194 (FIG. 1A, FIG. 1B; FIG. 9—Table 1, FIG. 10—Table 2). The results confirm up-regulation of miR-34a as a function of p53 status, and also point to strong up-regulation of miR-194 (FIG. 10—Table 2; FIG. 1A, FIG. 1B).

The significant up-regulation of miR-192 and miR-215 after p53 re-expression through Nutlin-3a treatment is of note because they are located, with miR-194, in two related microRNA clusters, the miR-194-2-192 cluster at 11q13.1 and the miR-215-194-1 cluster at 1q41.1 (FIG. 10—Table 2), and have the same seed sequence. miR-194 has the same mature sequence independently of cluster of origin, and miRNAs of the same cluster are usually expressed together. In the second chip array, expression of miR-192 and miR-215 together with miR-194 expression was observed (FIG. 1B; FIG. 10—Table 2). These data confirm the specificity of miR-194 up-regulation and show that both clusters participate in up-regulation. The inventors then determined the roles of miR-215-194-1 and miR-194-2-192 clusters in direct activation of p53.

p53 Induces Expression of miR-192, miR-194 and miR-215

To confirm the microarray data, the inventors first tested by q-RT-PCR for the presence of miR-34a, miR-194 and its cluster associates, miR-192 and miR-215, in WT TP53 compared to Mut TP53 cells (FIG. 12A).

WT TP53 cells retained higher expression of miR-34a, miR-194 and miR-192 (FIG. 12A), but did not show expression of miR-215 (showing that the 11q13.1 miR-194-2-192 cluster is associated with WT TP53 status in MM cells). To determine kinetics of activation of these miRNAs during p53 re-expression, the inventors treated MM1s cells with 10 μM Nutlin-3a at timepoints between 6 and 36 hr. After 6 hr of treatment, p53 was barely detectable by immunoblot analysis but increased by 12, 18 and 24 hr of treatment, to remain constant at 30-36 hr (FIG. 1C).

The non genotoxic activation of p53 in MM1s cells was also associated with MDM2 accumulation, CDKN1A (p21) expression and MYC down regulation after 12 hr of treatment (FIG. 1C).

To quantify kinetics of induction of a directly p53 responsive gene during the time course of treatment, CDKN1A mRNA expression was assessed by RT-PCR amplification (FIG. 1D). By northern blot and qRT-PCR analysis the inventors also studied kinetics of miRNA activation during p53 up-modulation in MM1s cells; kinetics of expression of miR-34a, miR194, miR-192 and miR-215 (FIG. 1E) were directly correlated with p53 protein up-regulation and p21 activation (FIG. 1C, FIG. 1D), while for another class of miRNAs, including miR-15 and miR-29a/b, which were used as negative control, the dynamics of expression appeared more related to downregulation of their repressor, Myc (FIG. 12B, FIG. 12C, FIG. 1D) than to p53 activation.

To confirm the responsiveness of selected miRNAs to p53, under various conditions, cell lines with varying TP53 status were treated with Nutlin-3a or vehicle (DMSO), followed by qRT-PCR, to monitor miRNA levels upon p53 activation (FIG. 13).

Specific activation of miR-34a, miR-192, miR-215 and miR-194 was detected only in the cell lines treated with Nutlin-3a and harboring WT TP53 (p<0.001) (FIG. 13).

Next, the inventors analyzed activation of these miRNAs after 12 hr of Nutlin-3a treatment of freshly isolated CD-138+ PCs (FIG. 14A), from 8 bone marrow aspirates of MM subjects. Two (Pt-1 and Pt-2) exhibited TP53 deletion by FISH analysis, while 6 (Pt-3 to Pt-8) retained TP53 genes (data not shown).

The inventors detected activation of p53 after 12 hr of Nutlin-3a treatment (FIG. 14B) in TP53 WT samples, in association with different levels of CDKN1A mRNA activation (FIG. 14C) and miR-34a, miR-192, miR-194, miR-215 up-regulation (FIG. 14D). Furthermore, to determine if p53 induction of these miRNAs was relevant in MM pathogenesis, the inventors analyzed the expression of miR-194, 192 and 215 in a panel of CD138+ PCs obtained from newly diagnosed MM subjects (n.33), MGUS (n.14) subjects and normal donors (n.4) (FIG. 23—Table 3) by qRT-PCR (FIG. 1F, FIG. 1G, FIG. 1H). By Kruskal-Wallis analysis the inventors found that these clusters of miRNAs are consistently down-regulated in MM samples (p<0.001) compared to MGUS samples. Altogether, these findings show that activation of p53 in MM cells mediates up-regulation of a specific set of miRNAs and that their down-regulation in primary tumor samples has important roles in MM progression.

Identification of the p53 Core Element in Pri-miR-192-194-2 Promoter at 11q13.1

To determine whether p53 is directly involved in the transcriptional regulation of miR-194-2-192 and miR-215-194-1 clusters, the inventors analyzed the cluster promoter regions. The upstream genomic region close to the transcription start site (TSS) (+1) of pri-miR-194-2-192 contains several highly conserved regions among human, mouse, rat, and dog sequences (from −162 to +21 with respect to the TSS).

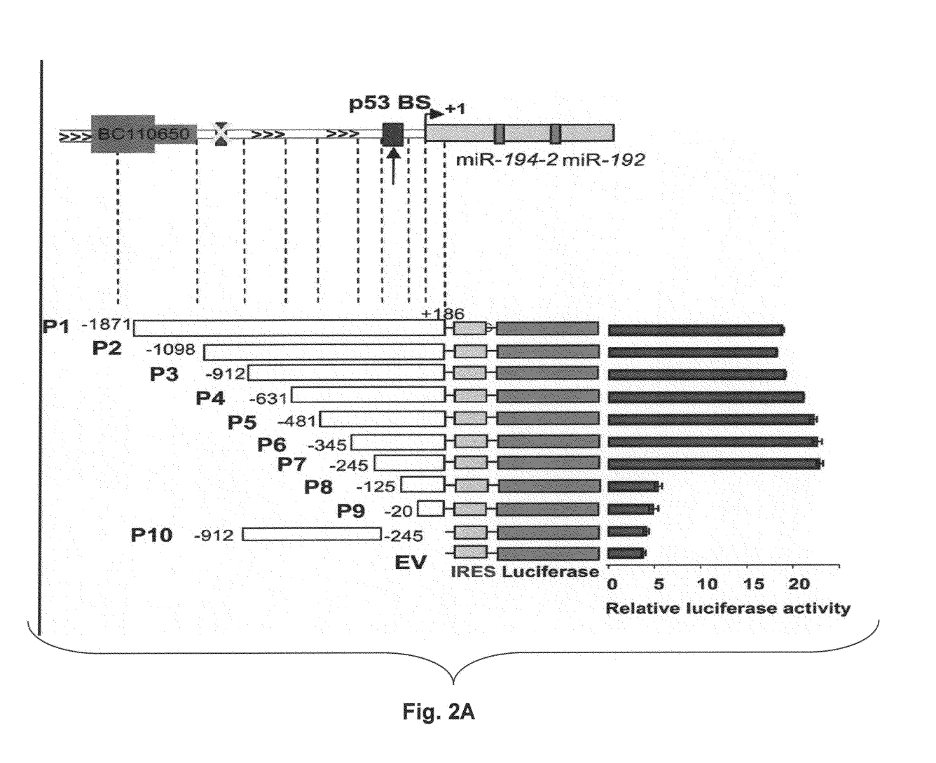

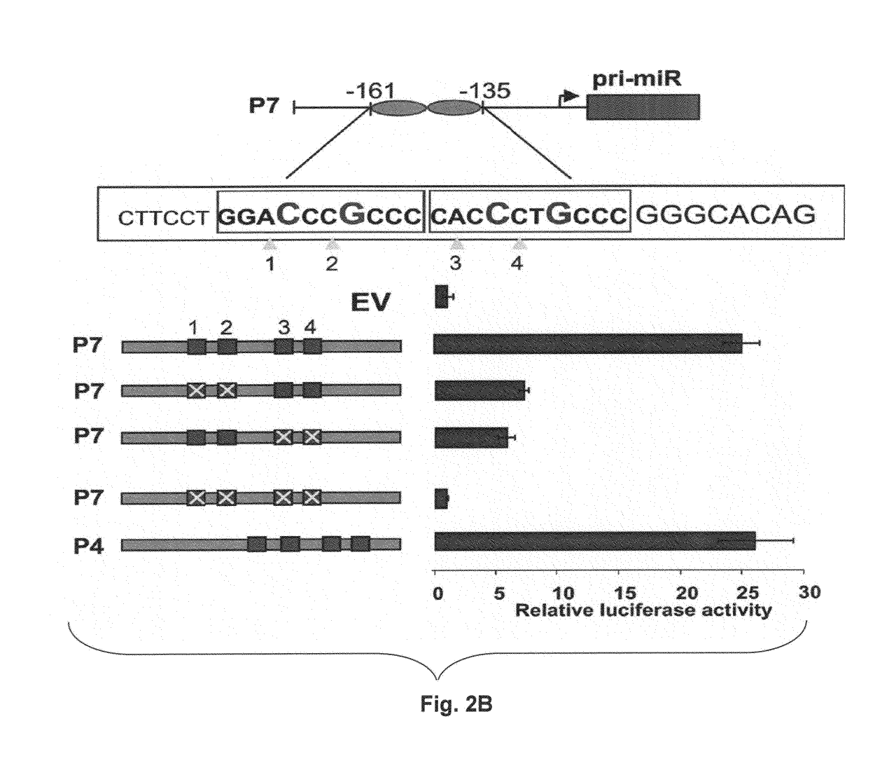

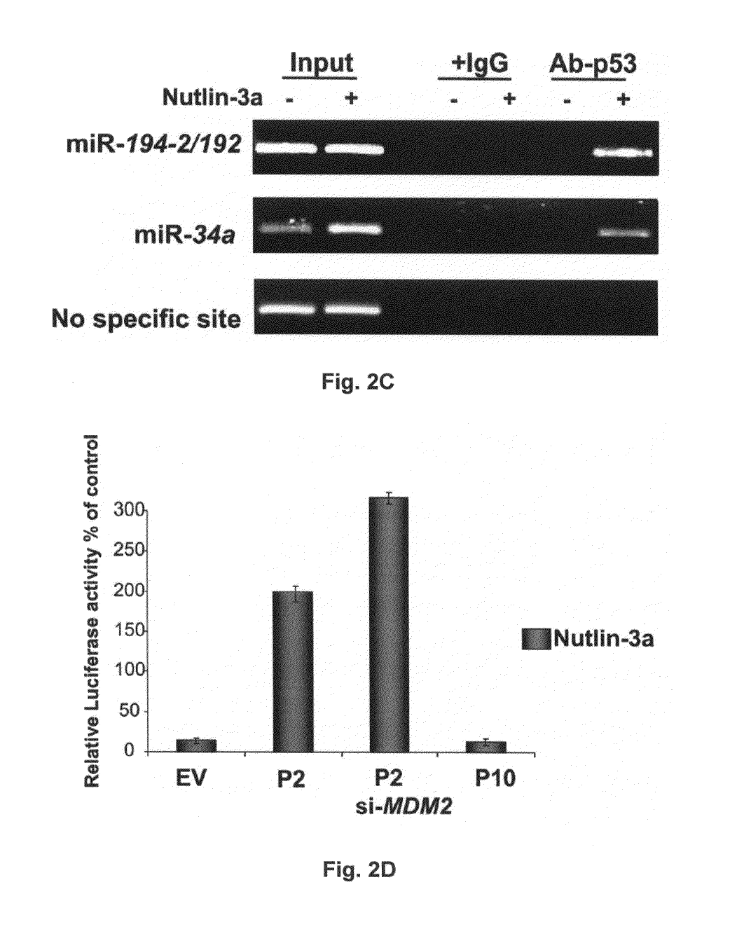

To identify the promoter region responsive to p53 re-expression, the inventors constructed reporter plasmids carrying various genomic sequences around the TSS of the pri-miR-194-2-192 cluster and subjected them to luciferase assay (FIG. 2A).

By bioinformatics search the inventors first identified a previously reported high score p53 consensus site between −900/−912 bp, but this site was not functional for p53 activation; luciferase reporter constructs excluding this region retained full activity (P3-P7) (FIG. 2A).

Results demonstrated that the region from −245 to +186 (P7) from the start of the pri-miR (+1) had promoter activity comparable to that of the longest regions in MM cells after forced expression of p53 (FIG. 2A), but regions from −125 to +186 (P8) and −912 to −245 (P10) did not cause luciferase activity. The inventors then identified a p53-responsive element between −245 and −125 by (FIG. 2B) because the construct excluding this region was not affected by p53 expression (FIG. 2A). Since conserved regions in a given gene promoter are expected to contain regulatory elements, the inventors focused on the highly conserved region controlling luciferase activity, the region between −161 and −135 bp. Luciferase assay using a construct mutated for each C and G contained in the two decamers of the hypothetical El-Deity consensus sequence revealed that this non predicted and previously unpublished region is critical for p53 transcriptional activation of pri-miR 194-2-192 cluster (FIG. 2B).

The inventors found that endogenous p53 physically interacts with the core element of pri-miR-194-2 promoter in MM1s cells, as demonstrated by ChIP assay after 12 hr of Nutlin-3a treatment (FIG. 2C). As positive control, the inventors used the p53 consensus site on the promoter of miR-34a, while a nonspecific sequence served as negative control (FIG. 2C). Furthermore, non genotoxic re-expression of p53 activated the promoter of both members of the pri-miR-194-2-192 cluster in MM1s cells (FIG. 2D).

To confirm the strong dependence of this promoter on p53 reactivation, MDM2 siRNA after p53 re-expression in MM1s cells led to higher relative luciferase activity. Taken together, these data show that p53 is a key transcriptional activator of pri-miR-194-2 through physical binding to the core promoter element (FIG. 2D). The inventors also attempted to identify the promoter and primary transcript of miR-215-194-1 on chromosome 1q41.1, but could not identify the primary transcript initiation point by 3′ and 5′ RACE and PCR amplification of the putative transcribed sequences (EST), although the inventors confirmed the previously published consensus site for p53 by Chip analysis (FIG. 15).

miR-192, miR-194 and miR-215 Affect p53-Dependent MM Cell Growth

To examine the relevance of p53-mediated regulation of miR-192, miR-194 and miR-215 in MM, the inventors first tested whether reintroduction of these miRNAs could affect the biology of MM cells.

miR-192, miR-194 and miR-215 were introduced by transfection in WT TP53 cell lines (MM1s, NCI-H929 and KMS28BM), as well as cells with mutated TP53 (RPMI8226), followed by detection of TP53 and mRNAs of target genes, CDKN1A and MDM2, by RT-PCR analysis (FIGS. 16A-16B).

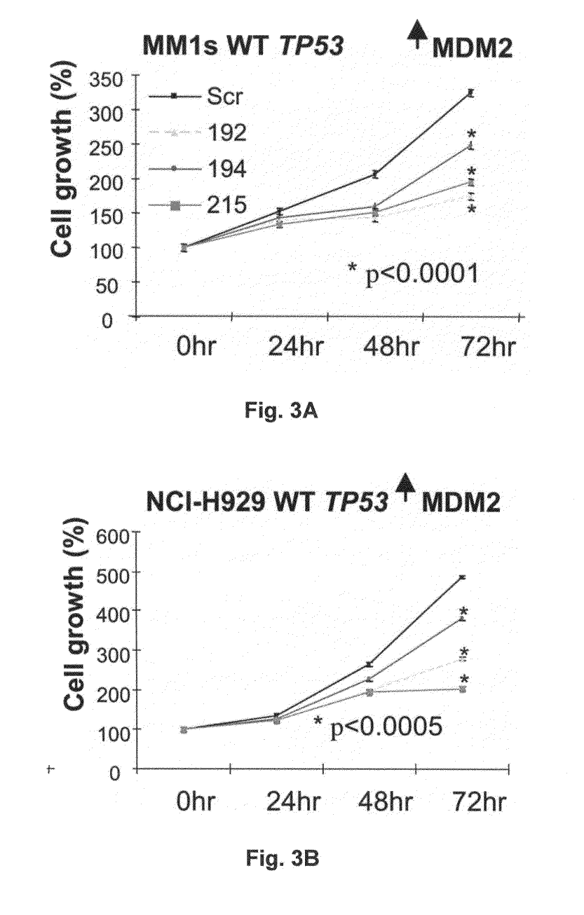

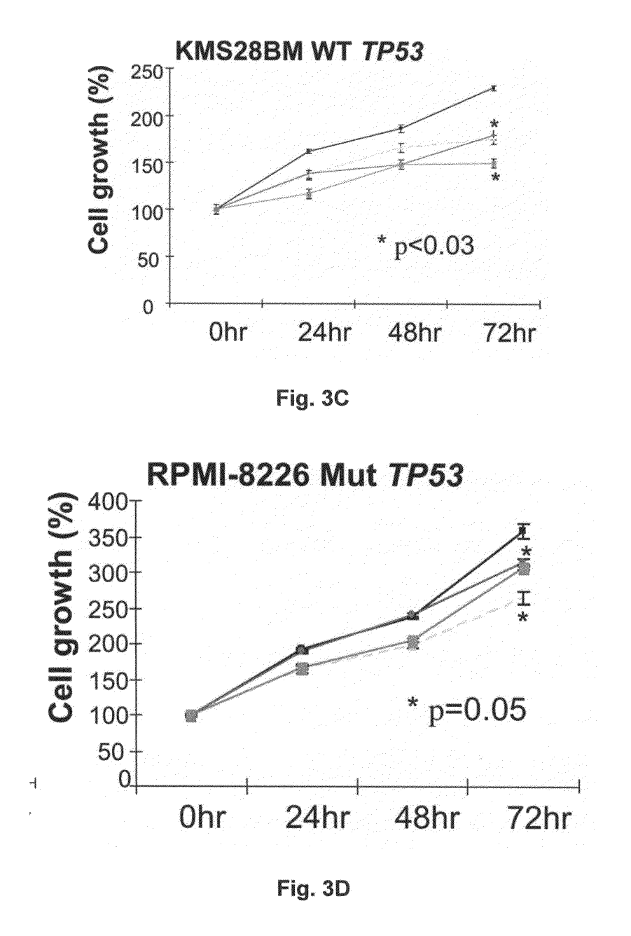

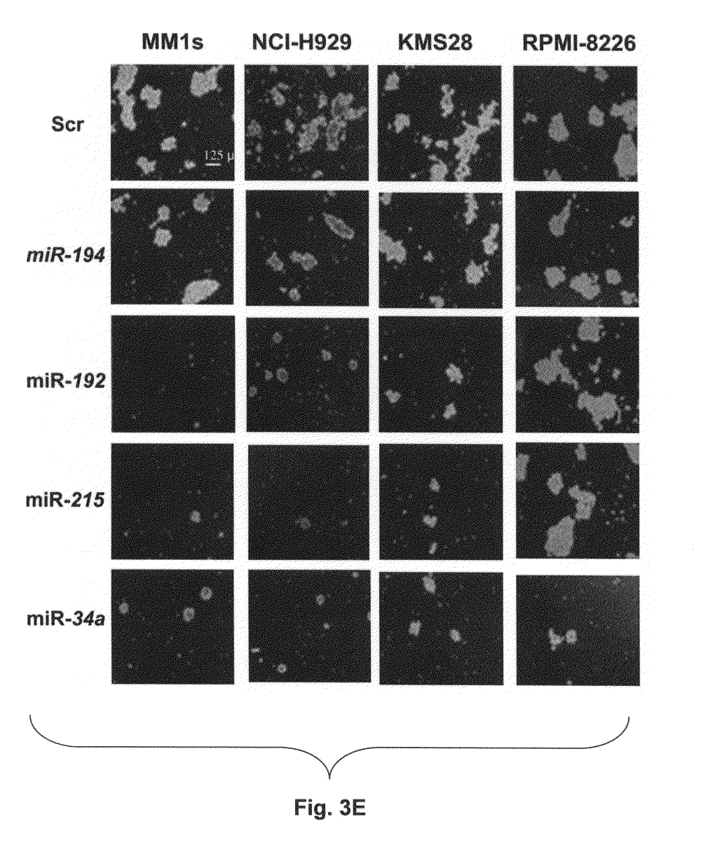

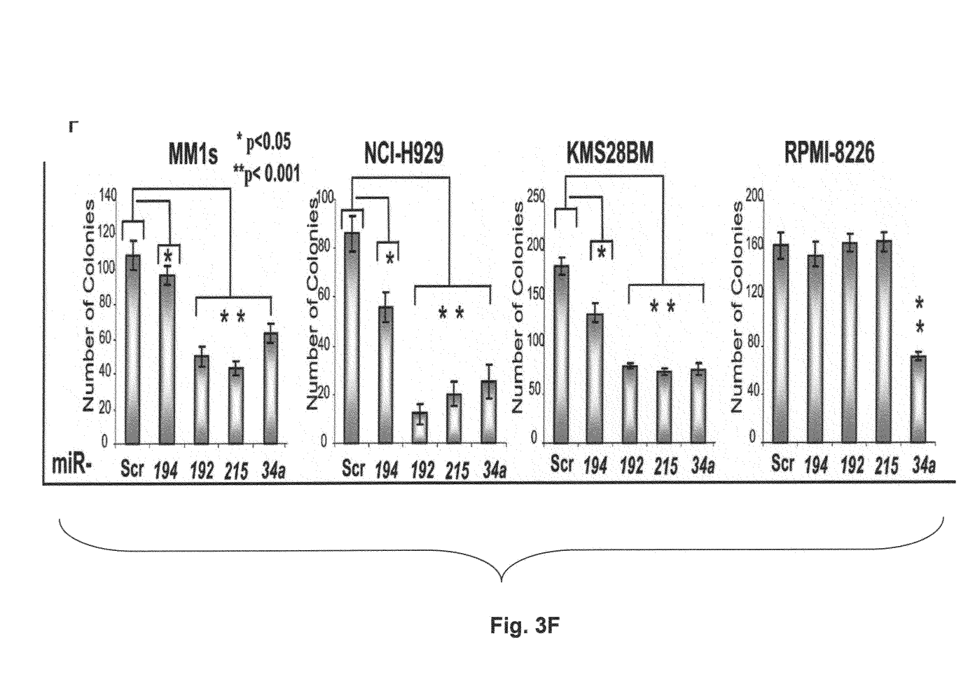

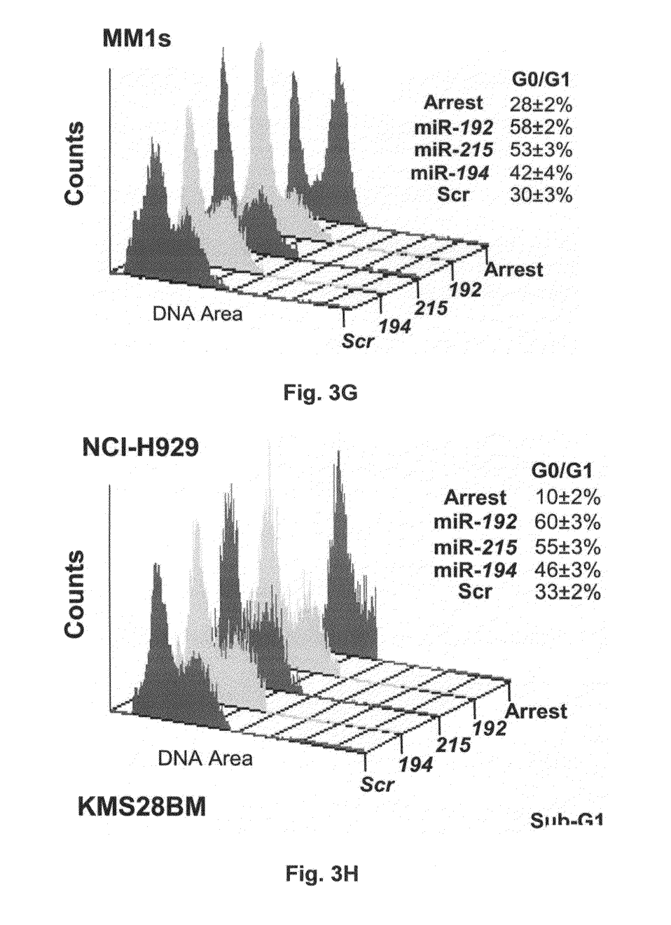

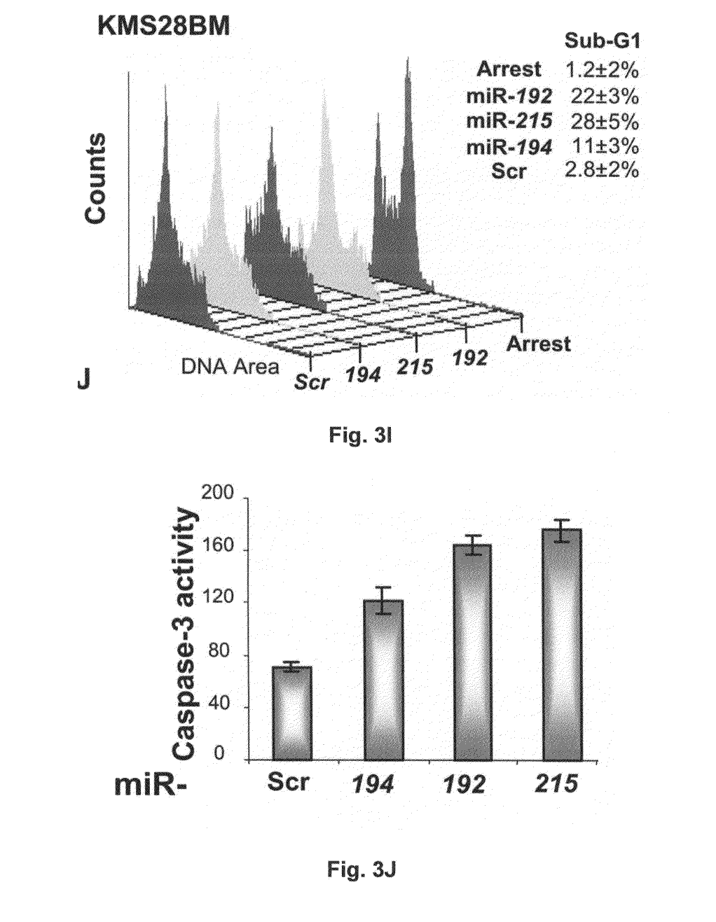

The inventors found consistent re-expression of CDKN1A in TP53 WT cells (FIG. 16A) after transfection but did not detect an increase in TP53 mRNA (FIG. 16B). Using MTS assay, the inventors observed significant growth arrest in the cells transfected with miR-192, miR-215 and a less significant arrest with miR-194 in MM cells carrying WT TP53 (FIG. 3A, FIG. 3B, FIG. 3C), as compared to scrambled sequences; in contrast, the inventors did not detect this effect in RPMI-8226 cells (FIG. 3D) expressing mutant TP53.

Next, the inventors determined whether p53 responsive miRNAs might interfere with clonigenic survival of MM cells. MM cells were lentivirus-transduced with miR-192, miR-215, miR-194 and miR-34a; miR-192 and 215 in WT p53 cells suppressed colony formation to an extent comparable to miR-34a, which was used as an internal control. Of note, miR-194 was less effective than miR-215 and miR-192. These miRNAs did not suppress colony formation in RPMI-8226 (FIG. 3E and FIG. 3F) or U266 cells (unpublished data), while miR-34a did exhibit colony suppression in these mutant TP53 cells, confirming its p53 independent apoptotic action.

To further explore the p53-dependent mechanism(s) of miR-192, miR-215 and miR-194 interference with cell growth and colony formation, flow cytometry was used to determine if their expression affects progression through the cell cycle.

In the two WT TP53 cell lines with high expression of MDM2 mRNA, MM1s and NCI-H929 (FIG. 11B), the p53-responsive miRNAs induced a severe G0/G1 arrest; arrest was observed in ˜30% of scrambled-transfected cells vs ˜60% of the cells transfected with miR-192 and miR-215 and ˜45% for miR-194 (FIG. 3G, FIG. 3H). Instead, in KMS-28BM cells, retaining WT TP53 but expressing lower levels of MDM2 mRNA (FIG. 11B), at 48 hr after transfection, the inventors detected increases of sub-G1 fractions (indicative of cell death) in cells transfected with miR-192 (˜25% sub-G1), miR-215 (30%) and miR-194 (12%), compared with ˜3% in control cells transfected with Scr sequence (FIG. 31). At 48 hr after transfection, the inventors also detected increased caspase-3 activity (FIG. 3J).

The differential effect of the miRNAs on TP53 WT cells carrying lower MDM2 mRNA basal expression (FIG. 11B) led the inventors to analyze MDM2 levels after miRNA transfection (FIG. 16C). MDM2 mRNA, but not protein, was detected after MM cell transfection, because MDM2 protein is rapidly auto-ubiquitinated and degraded through the proteasome pathway, guaranteeing a short half-life; p53 induction is necessary for its clear detection in MM cells.

In only one MM cell line (Mut TP53 RPMI-8226) of six analyzed, was MDM2 protein detected without p53 activation (FIG. 11A, FIG. 13C). The inventors also noted that MDM2 mRNA was down-regulated after ectopic expression of these miRNAs, principally in WT TP53 cells, but to some extent in Mut TP53 cells (RPMI-8226) (FIG. 16C).

These data were confirmed at the protein level in RPMI-8226 cells where the inventors observed ˜20% down regulation of MDM2 protein at 72 hr after miRNA transfection (FIG. 16D).

These results show that ectopic expression of miR-192, miR-215 and miR-194 in WT TP53 cells inhibits cell growth and enhances apoptosis, effects that are now believed to be related to MDM2 regulation in MM cells.

Human MDM2 is a Direct Target of miR-192, miR-194 and miR-215

The data demonstrate that miR-192, miR-215 and miR-194 biological function in MM cells is p53-dependent. After introduction of these miRNAs, TP53 mRNA level did not change in MM cells but higher CDKN1A and lower MDM2 mRNA levels were observed (FIG. 16A).

Both genes, MDM2 and CDKN1A, are direct targets of p53 but their expression in this case was not preceded by TP53 transcription (FIG. 16B).

Thus, the inventors herein now believe that miR-192, miR-194 and miR-215 target expression of MDM2. To further examine effects of these miRNAs on MDM2 protein expression in WT TP53 MM cells (MM1s and NCI-H929) where MDM2 protein was not detectable without p53 activation (FIG. 1C, FIG. 13A), the inventors analyzed the consequences of ectopic expression of miR-192, miR-194 and miR-215 at 72 hr after transfection and 12 hr of non genotoxic activation of p53 by Nutlin-3a (10 μM).

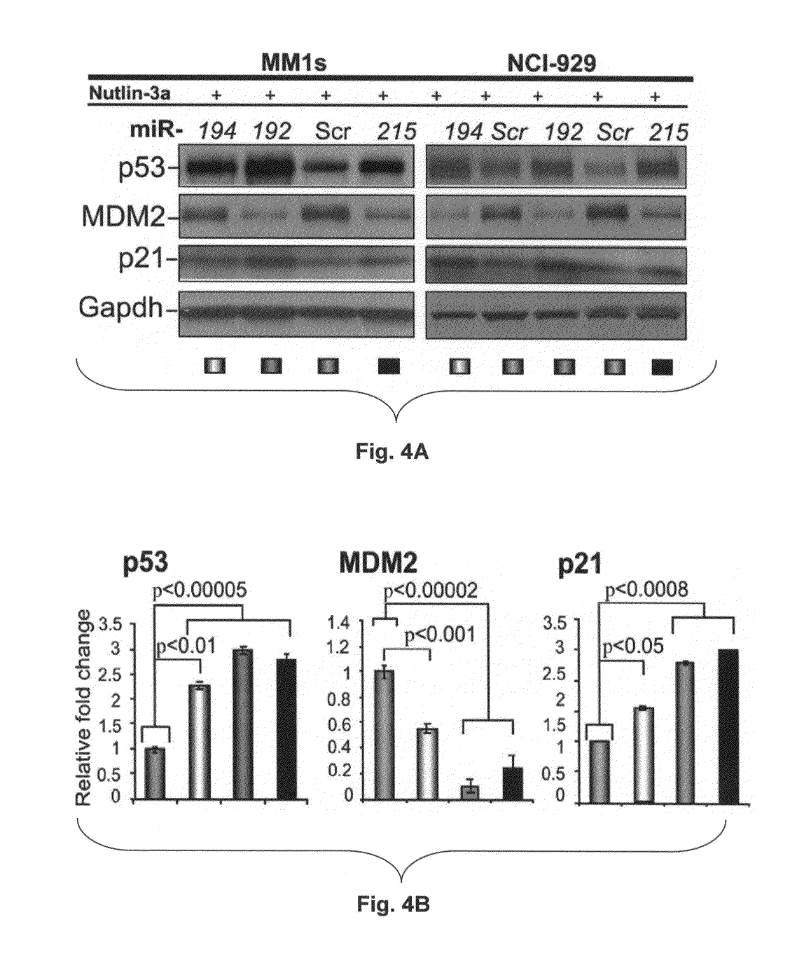

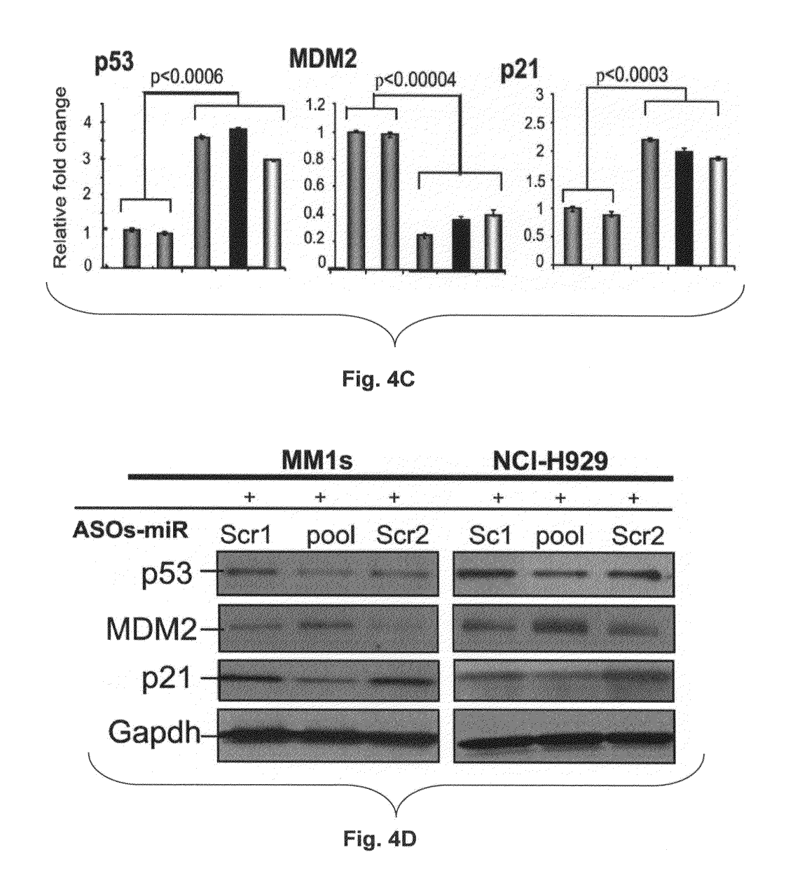

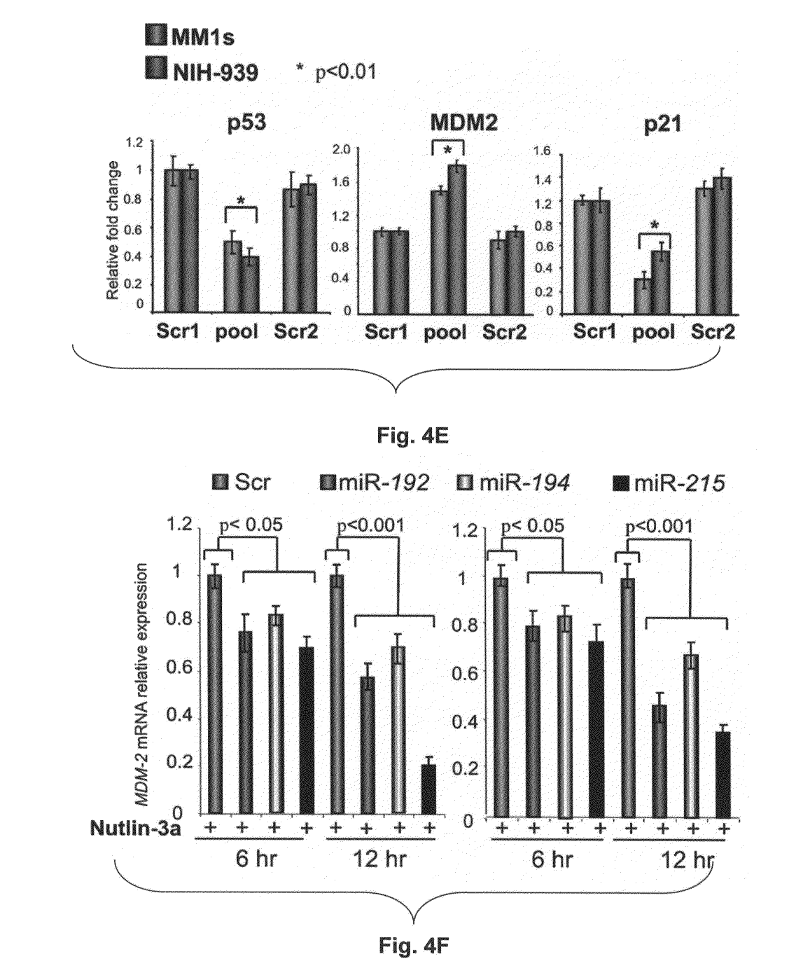

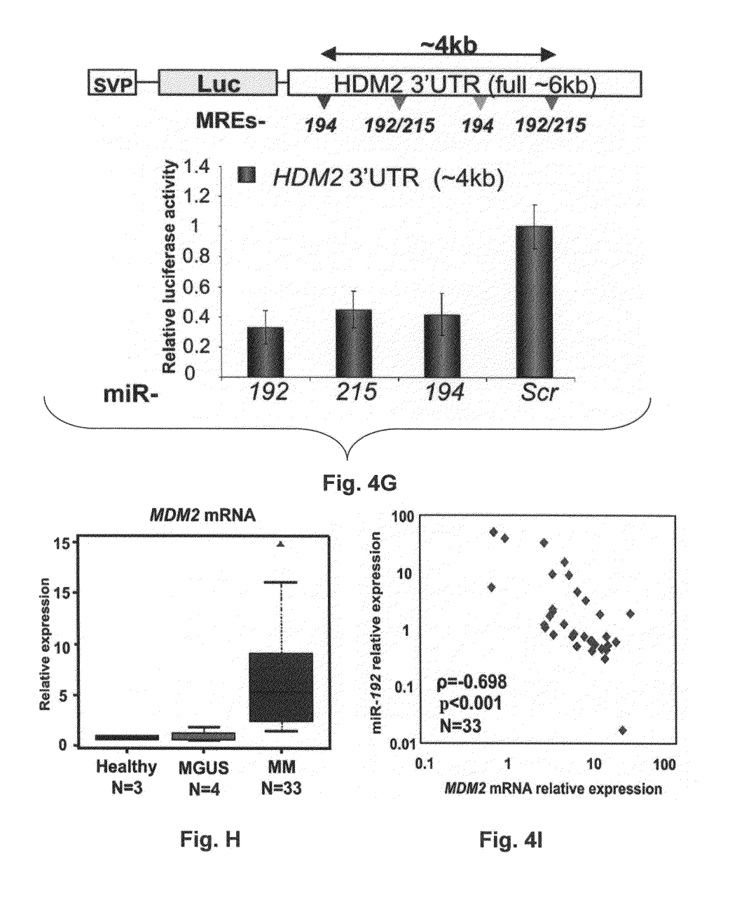

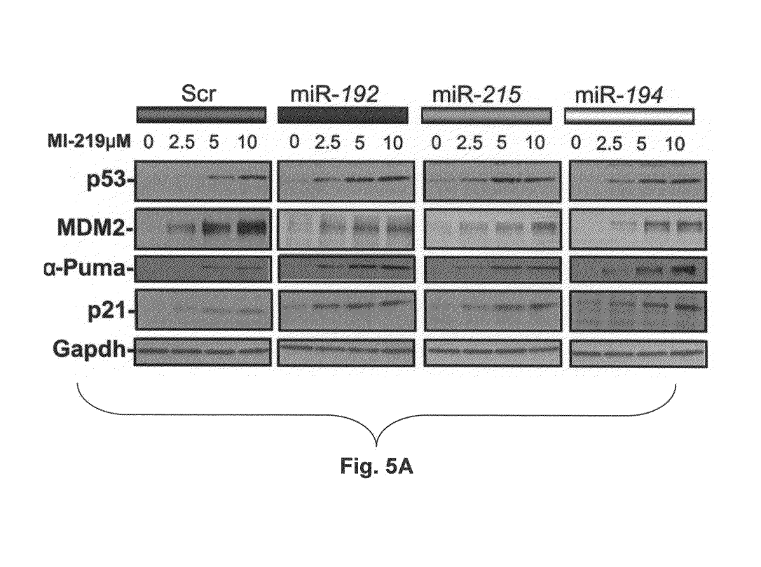

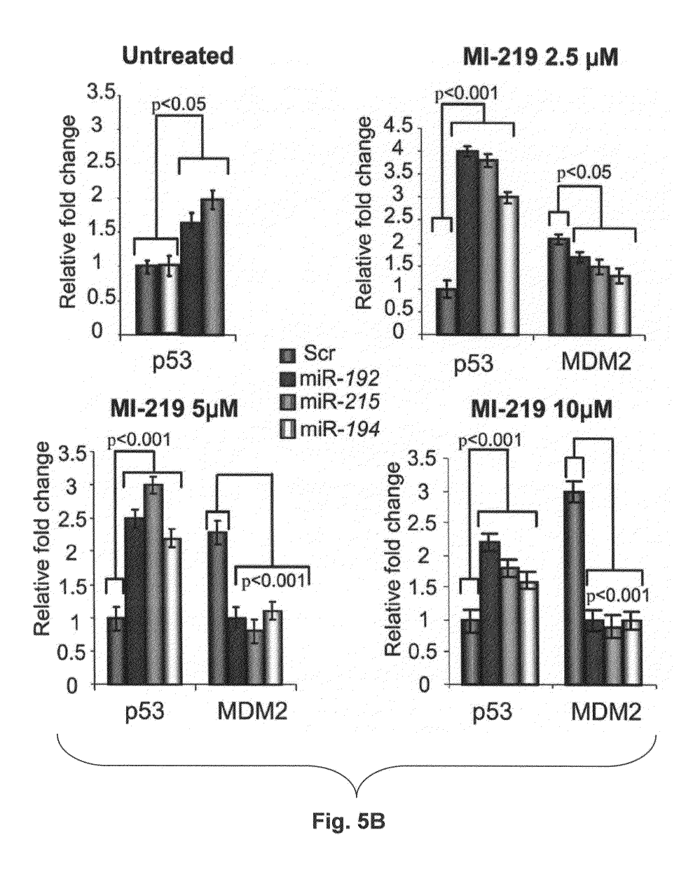

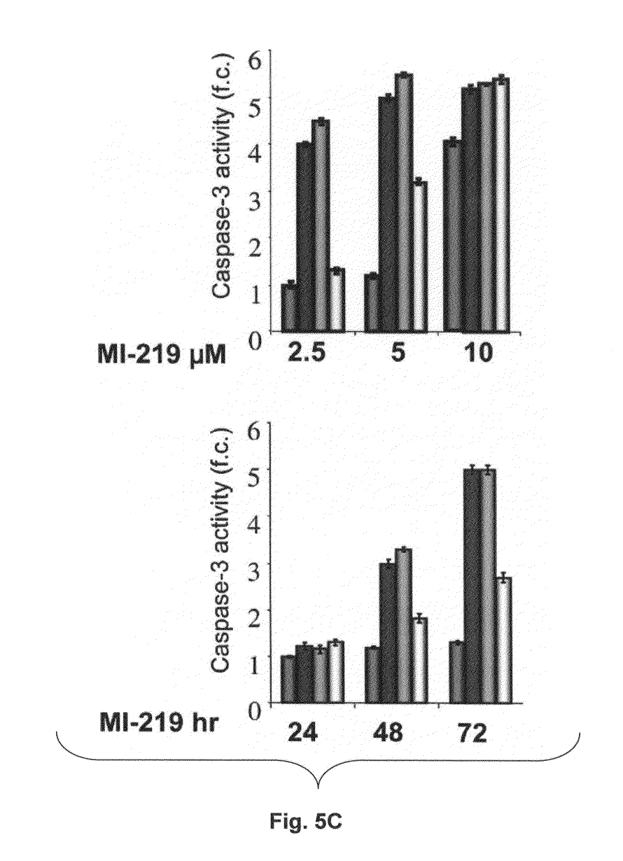

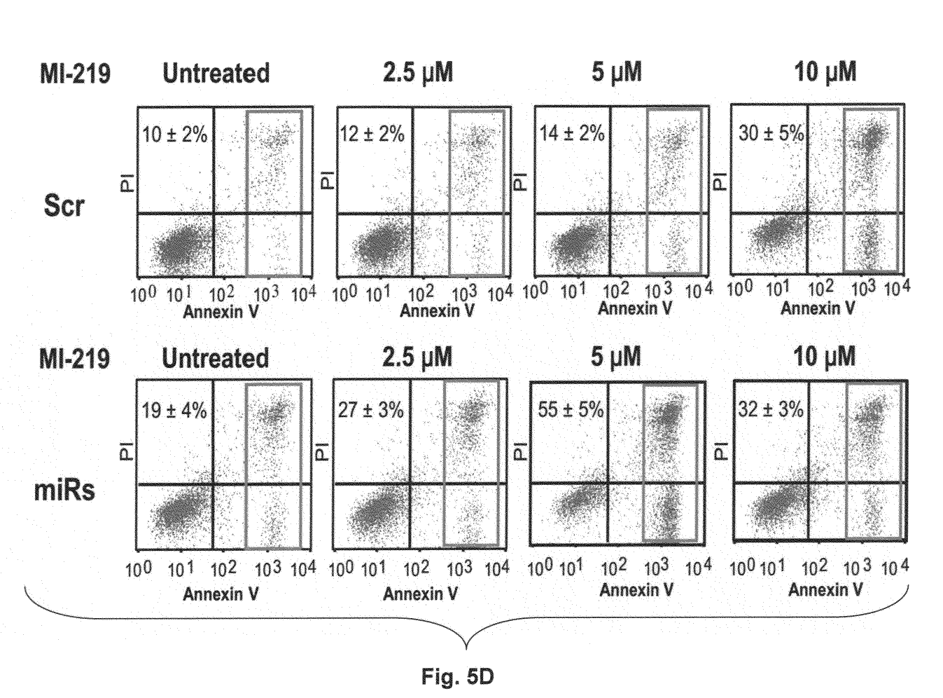

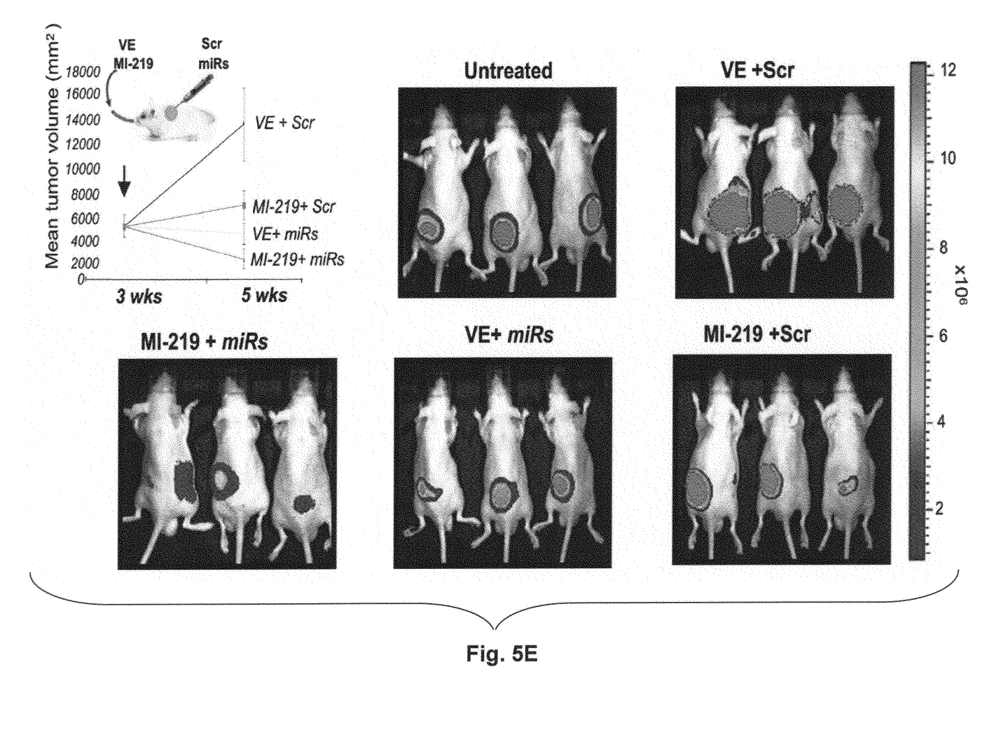

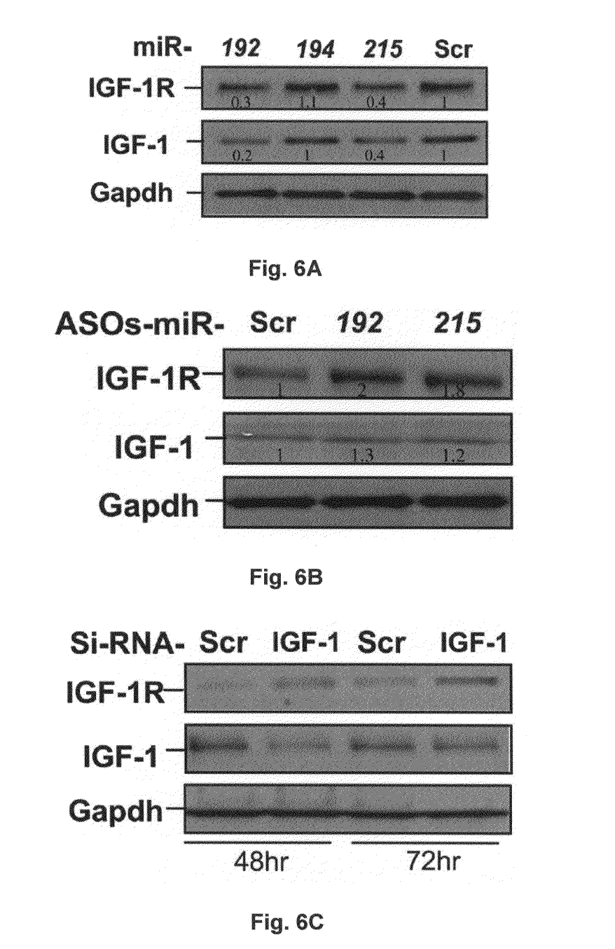

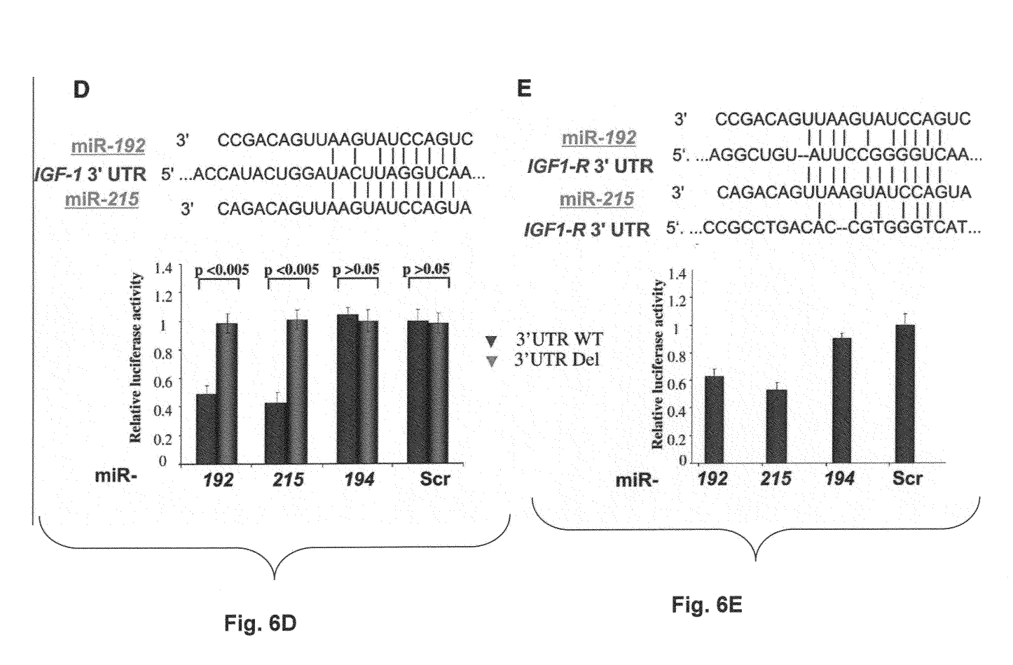

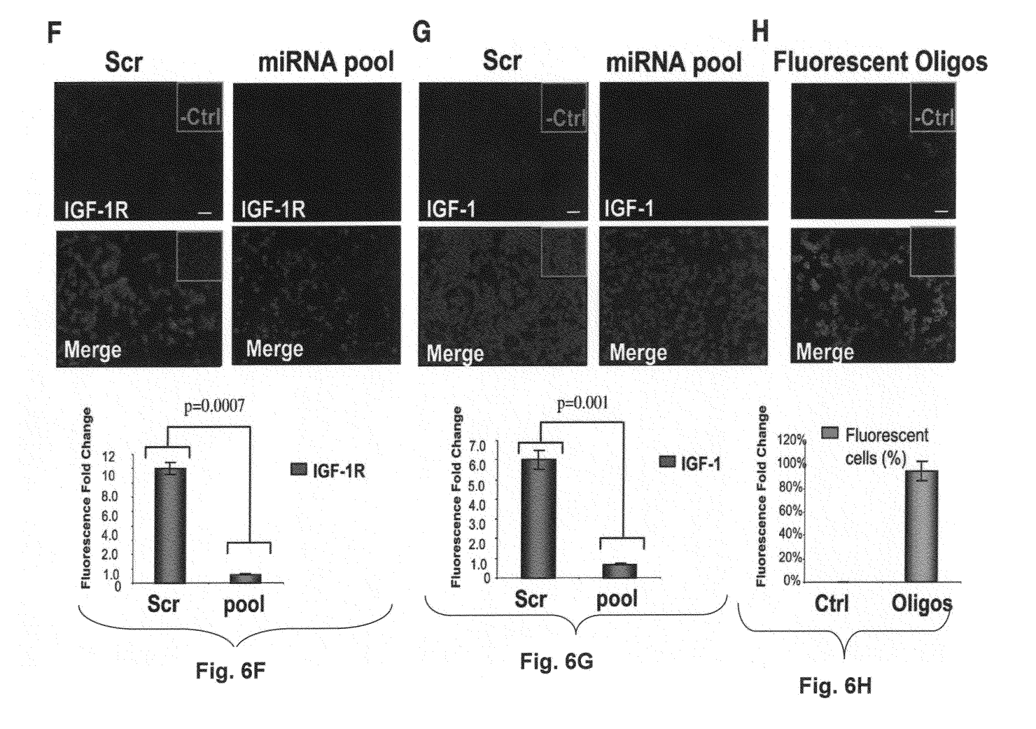

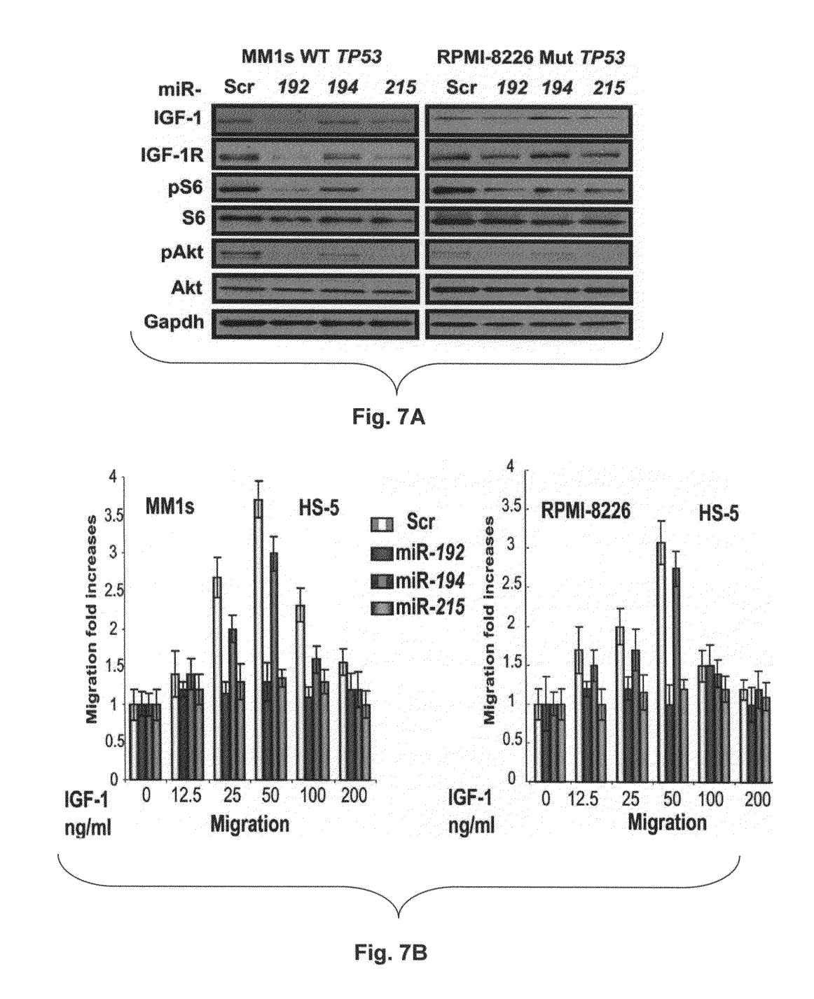

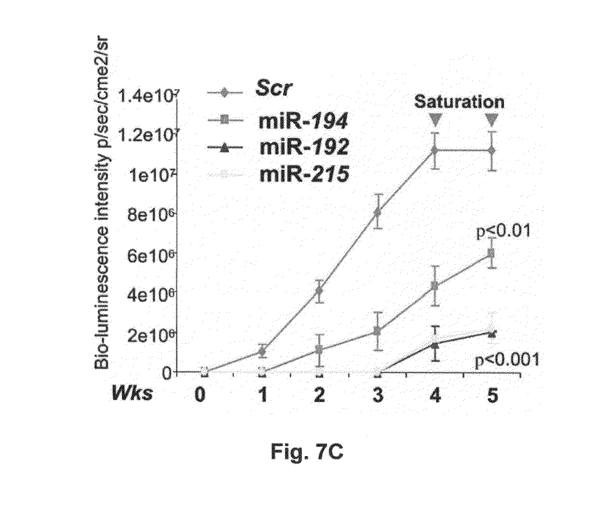

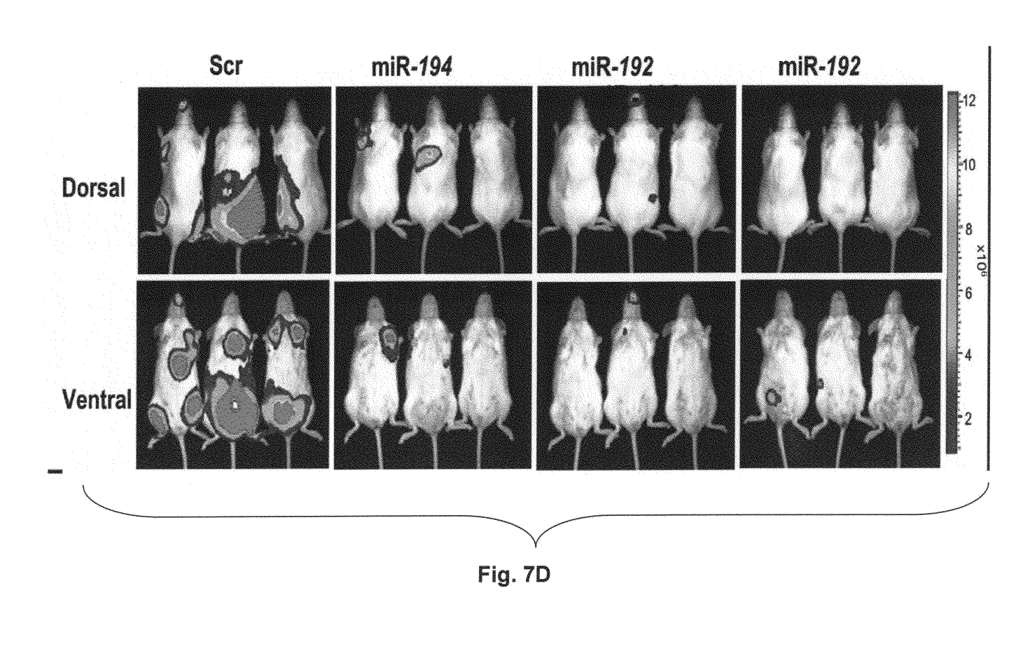

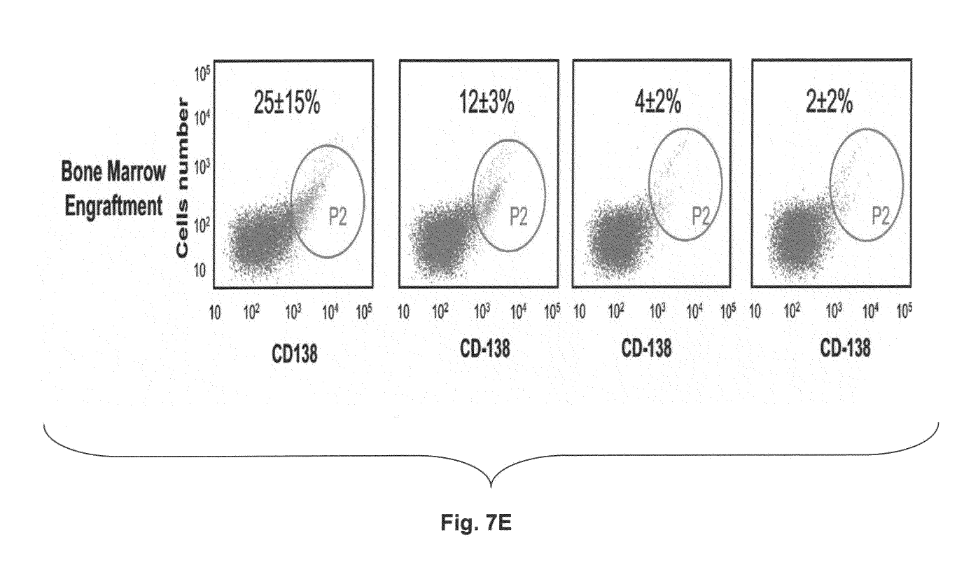

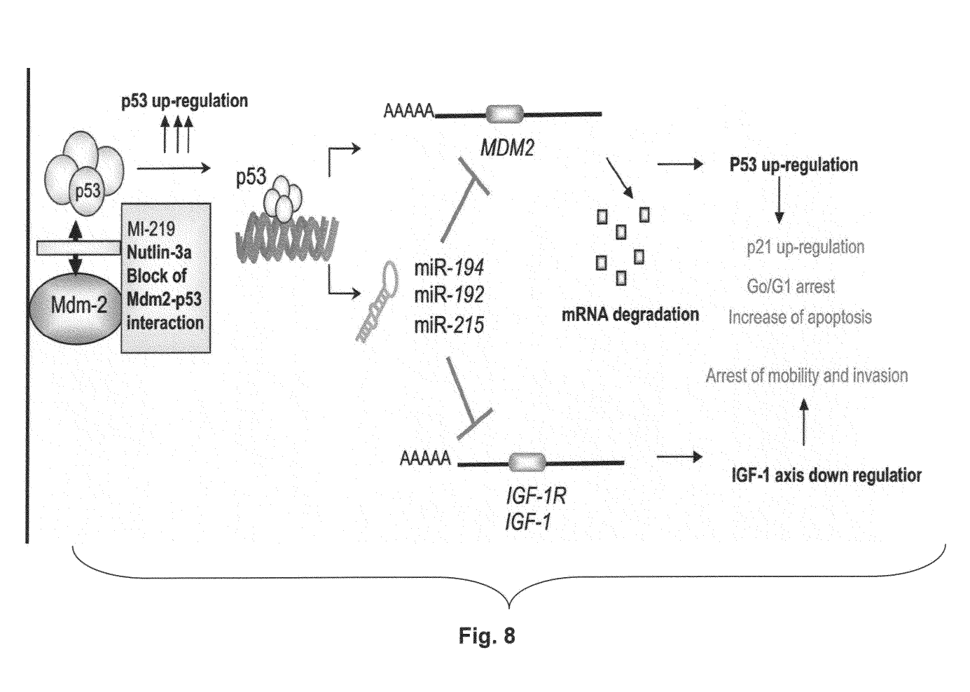

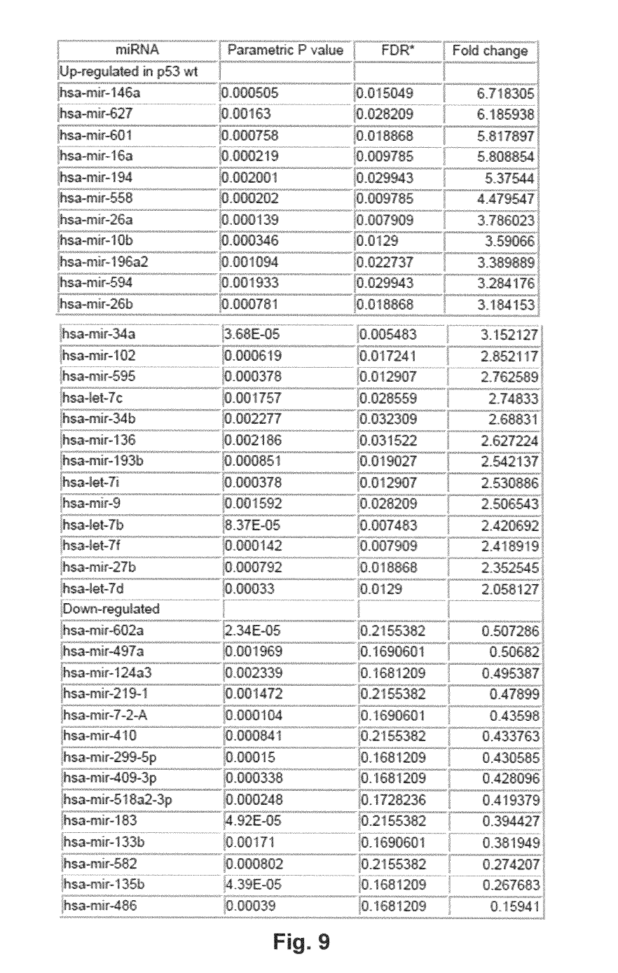

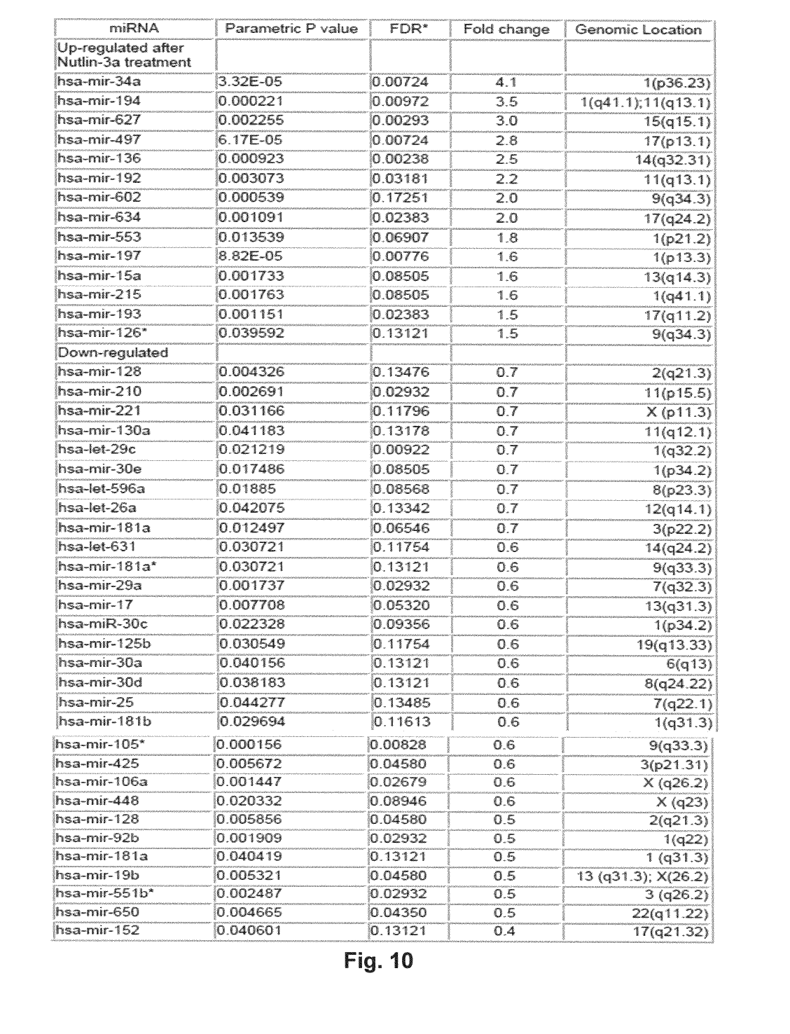

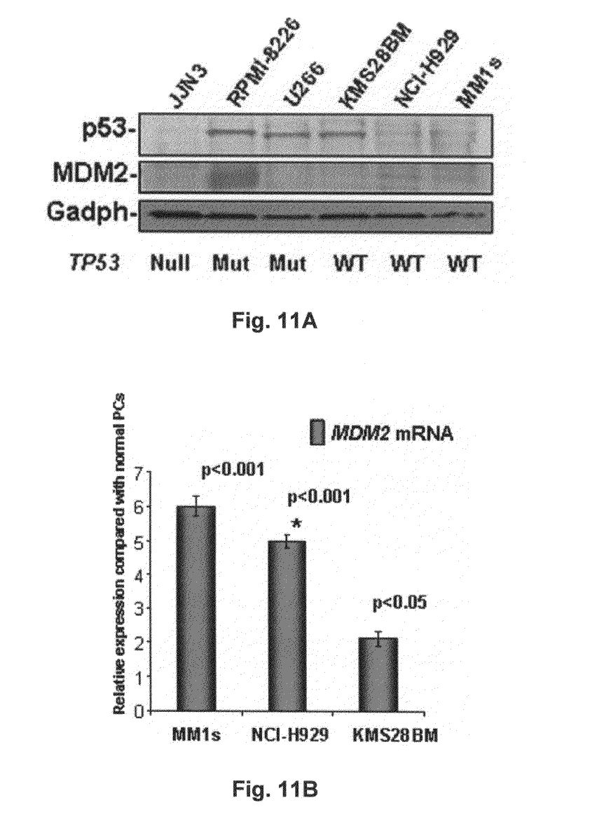

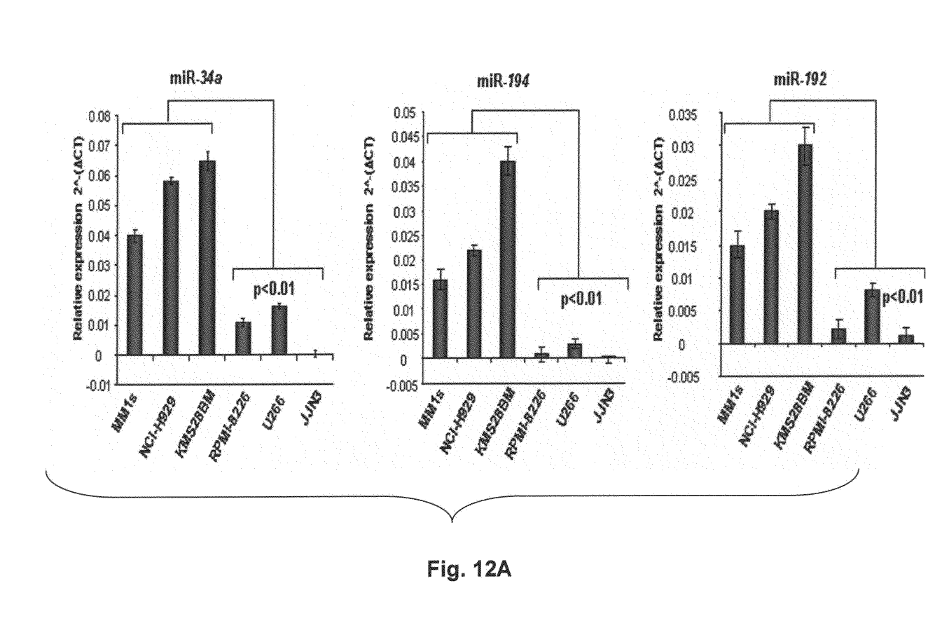

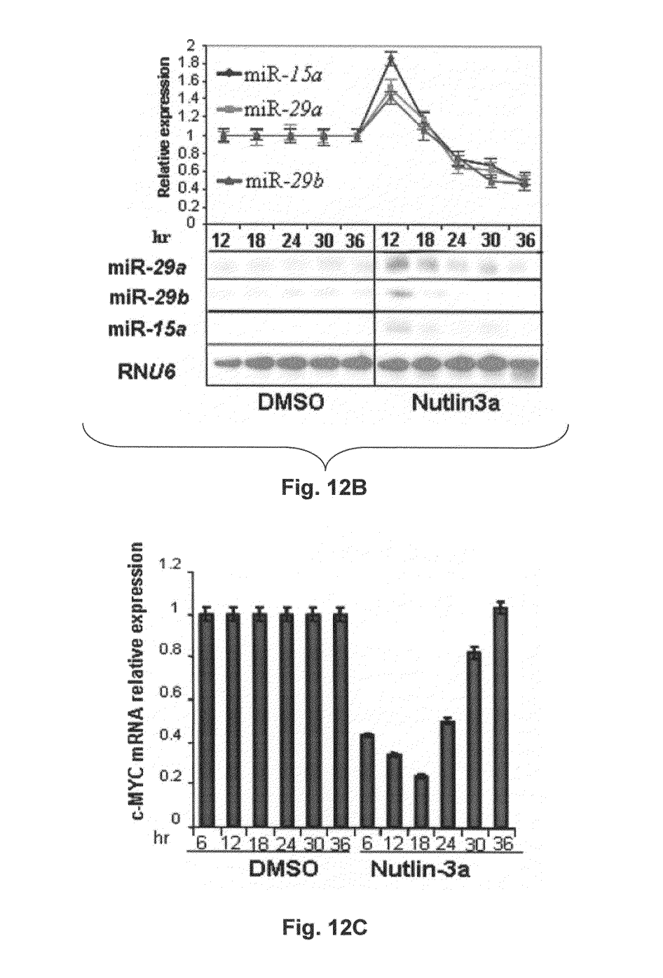

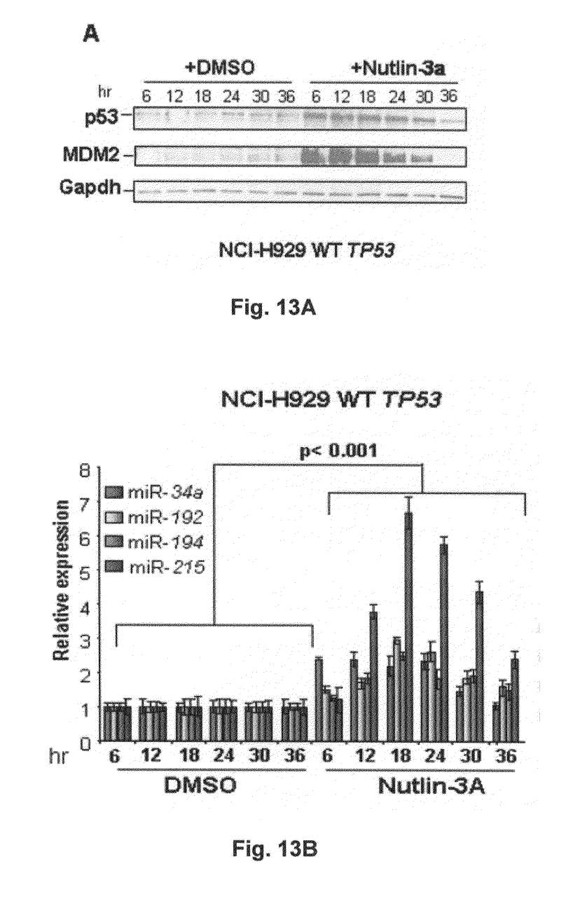

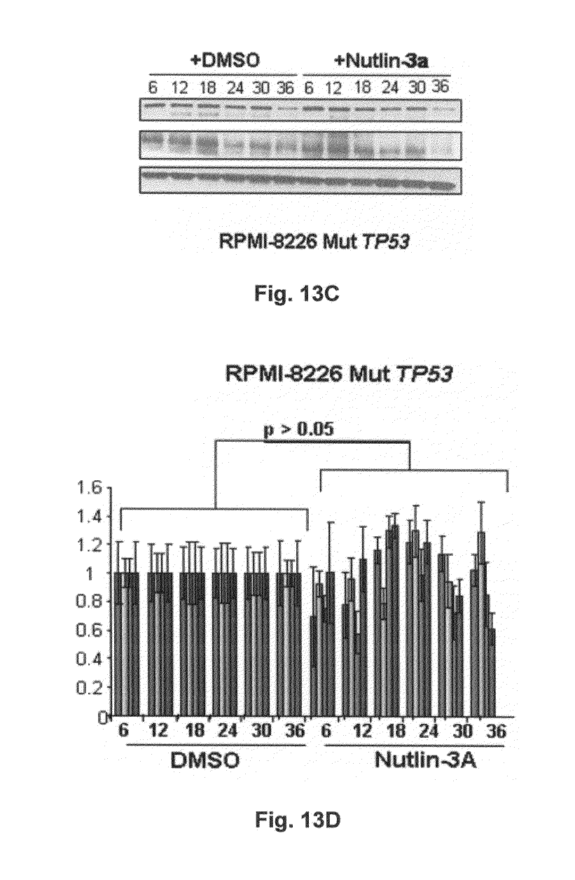

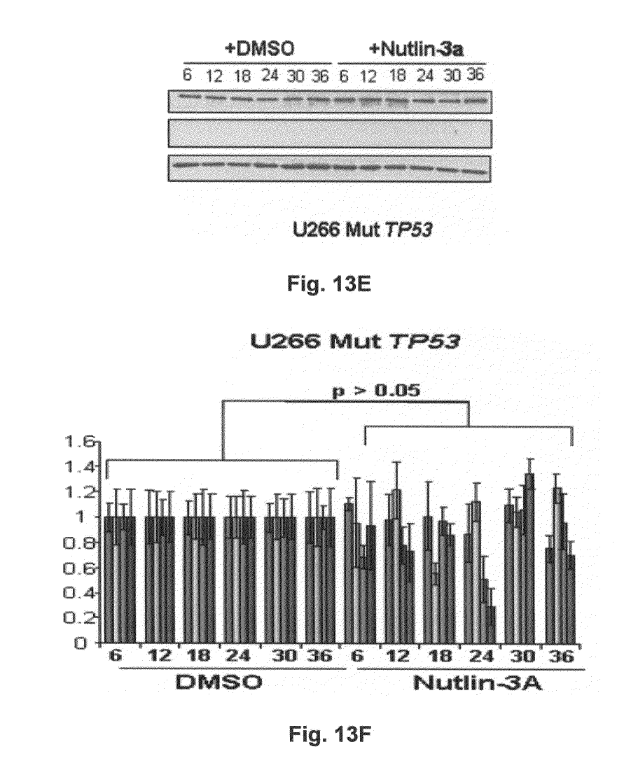

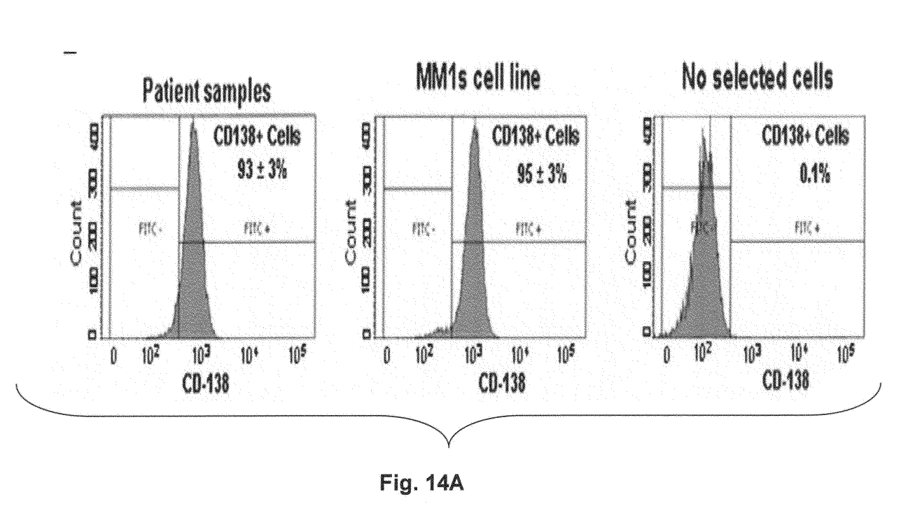

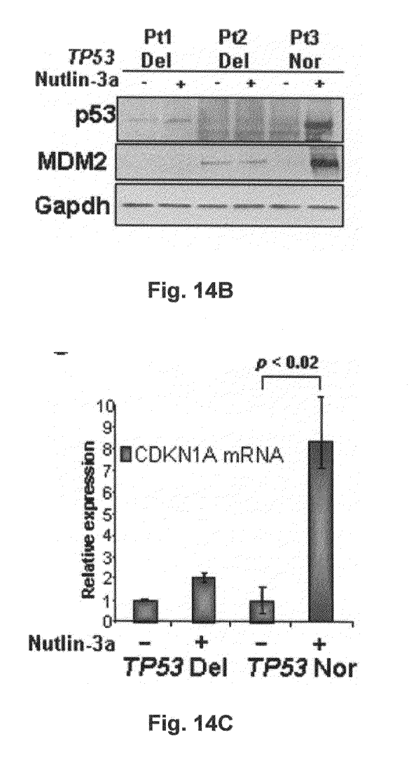

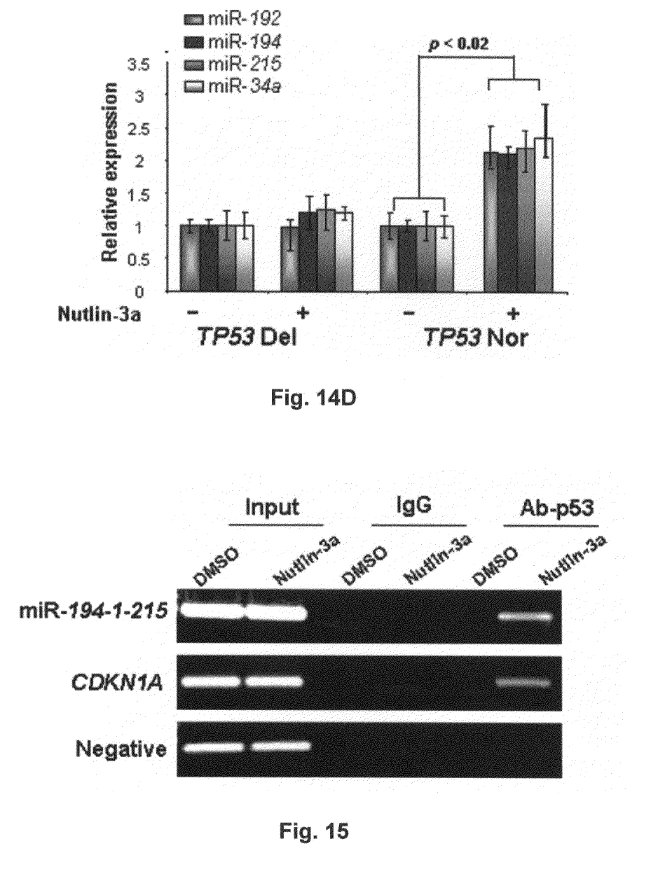

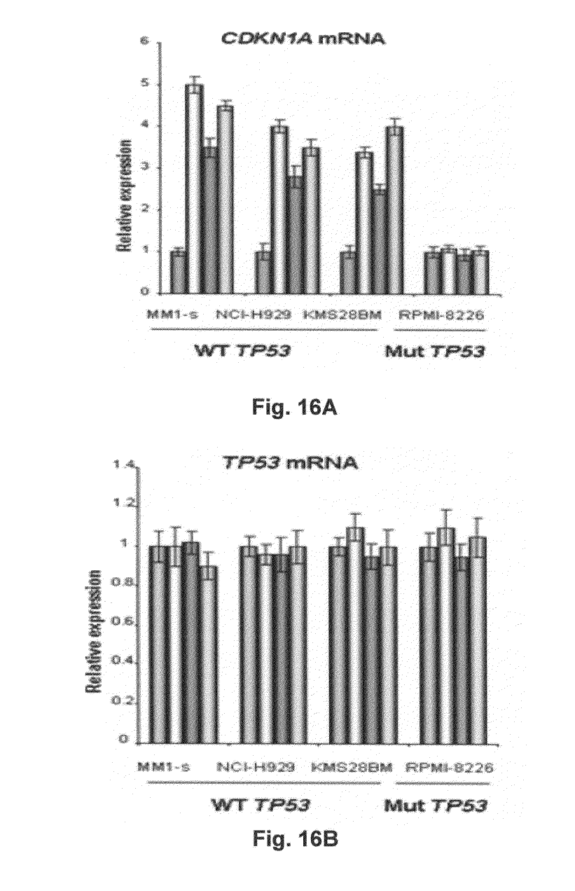

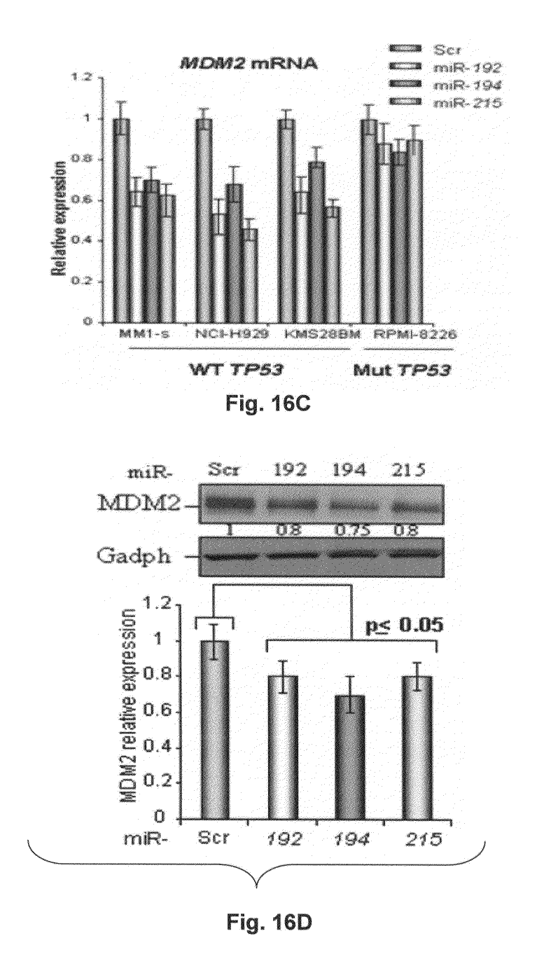

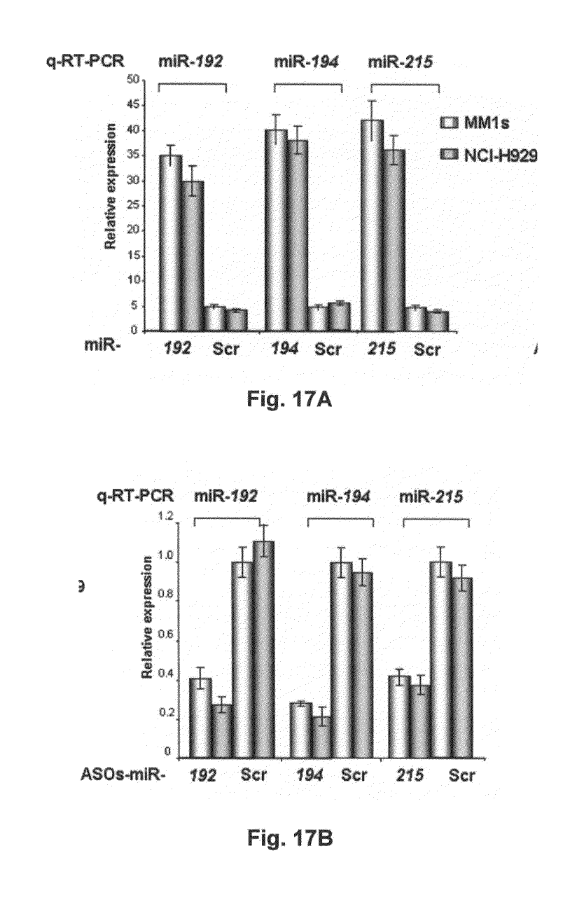

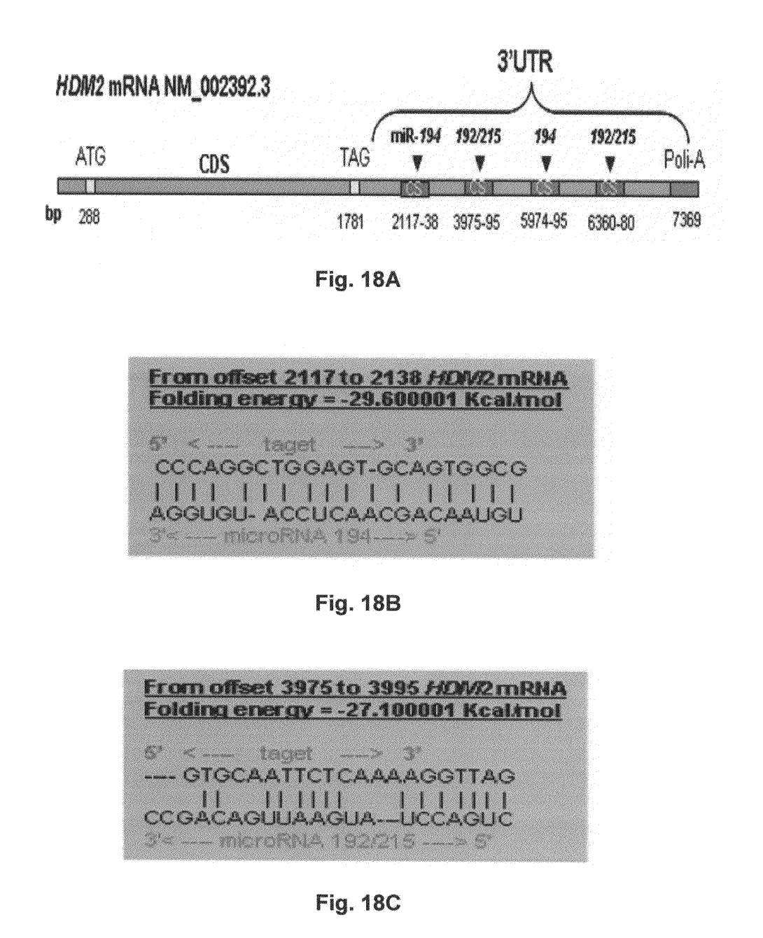

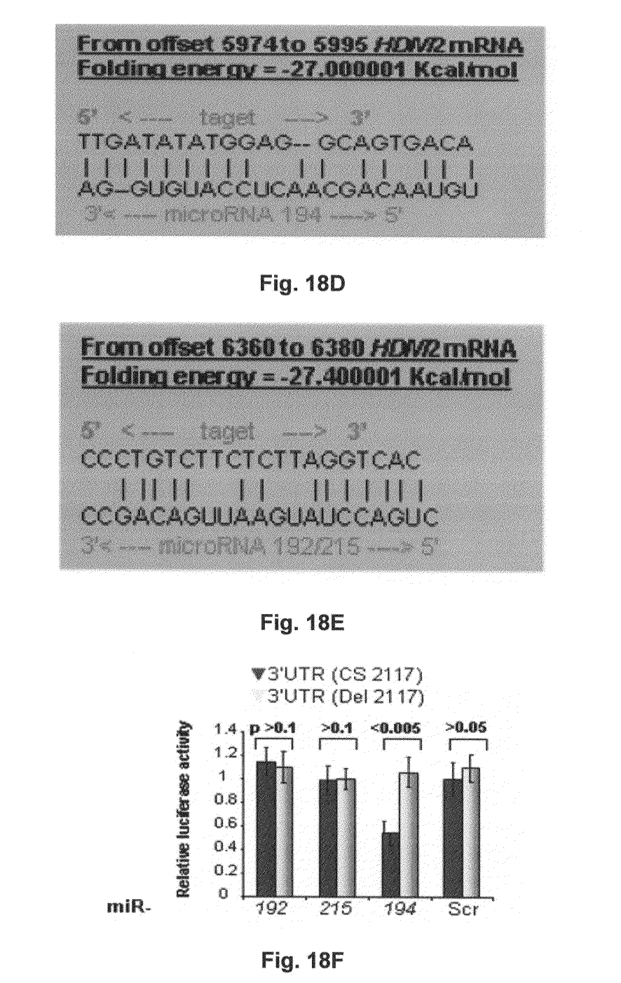

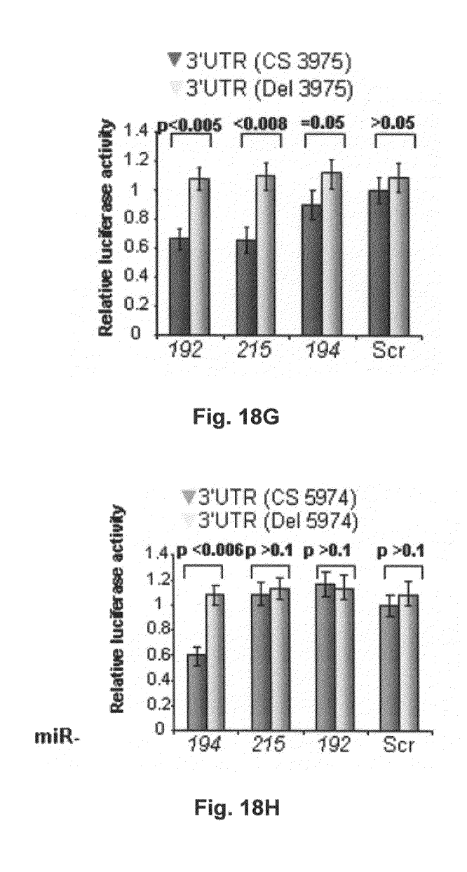

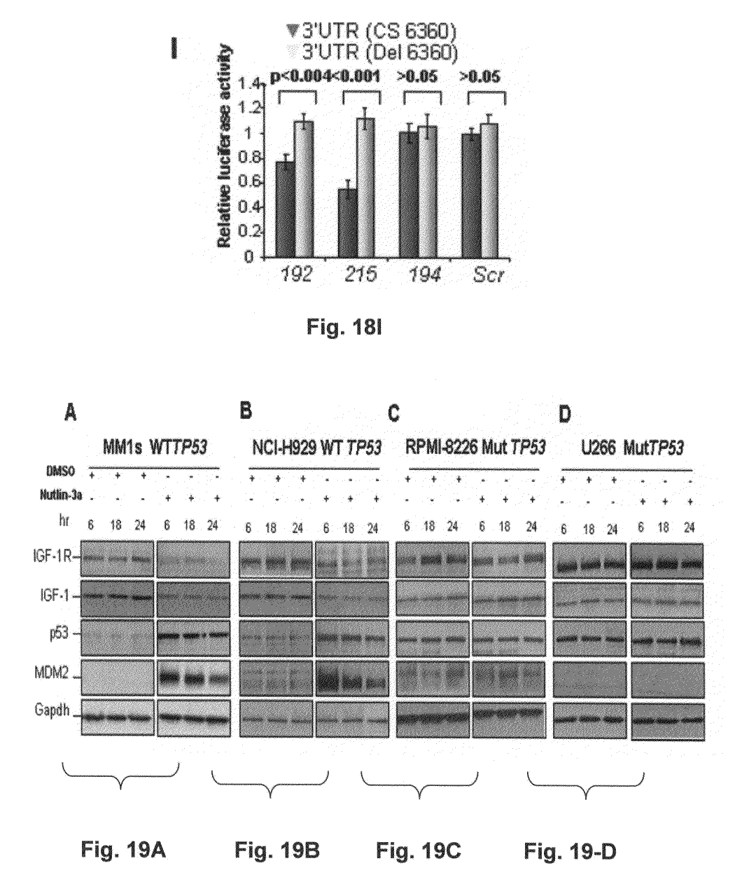

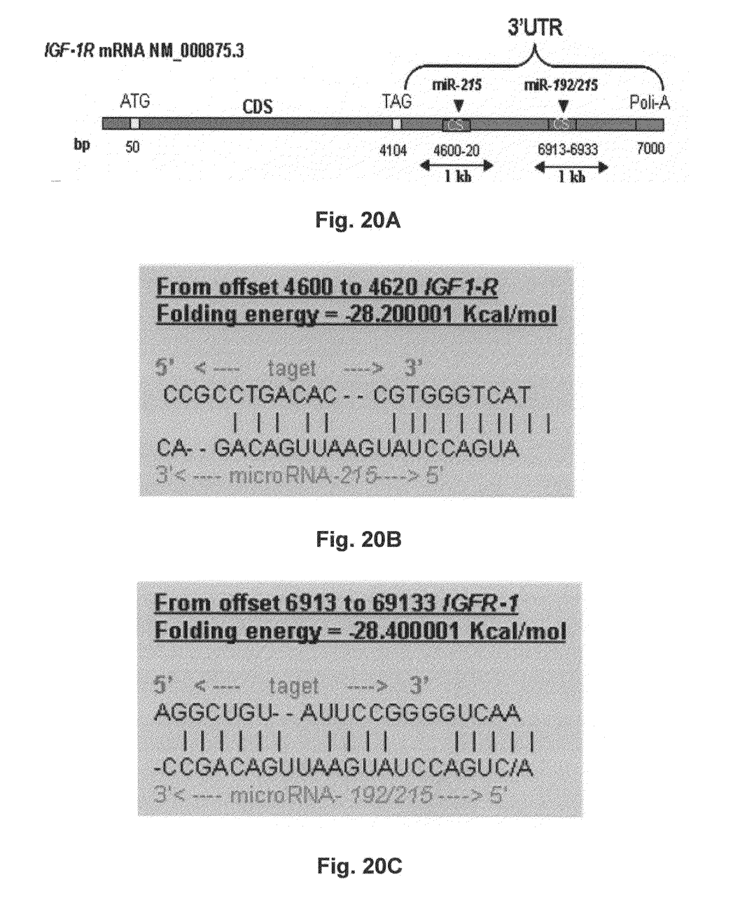

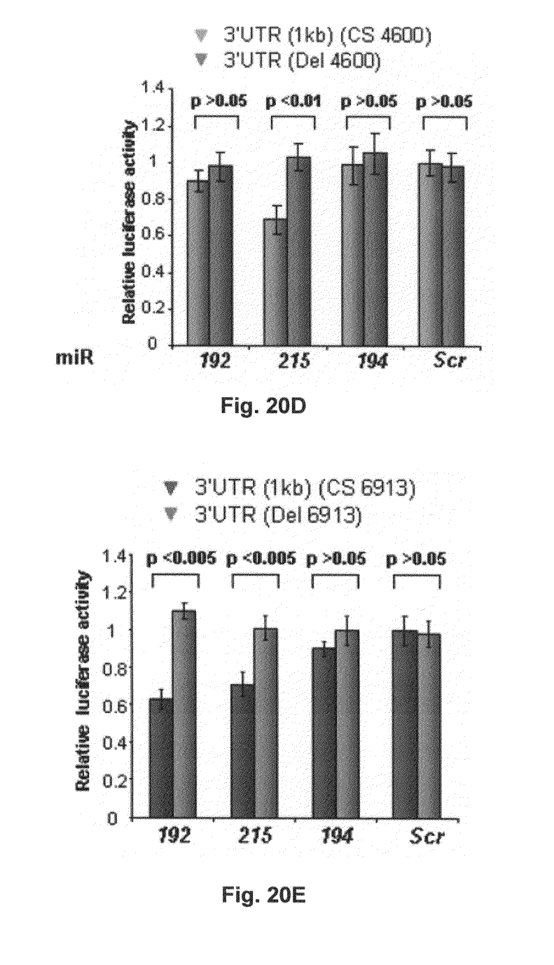

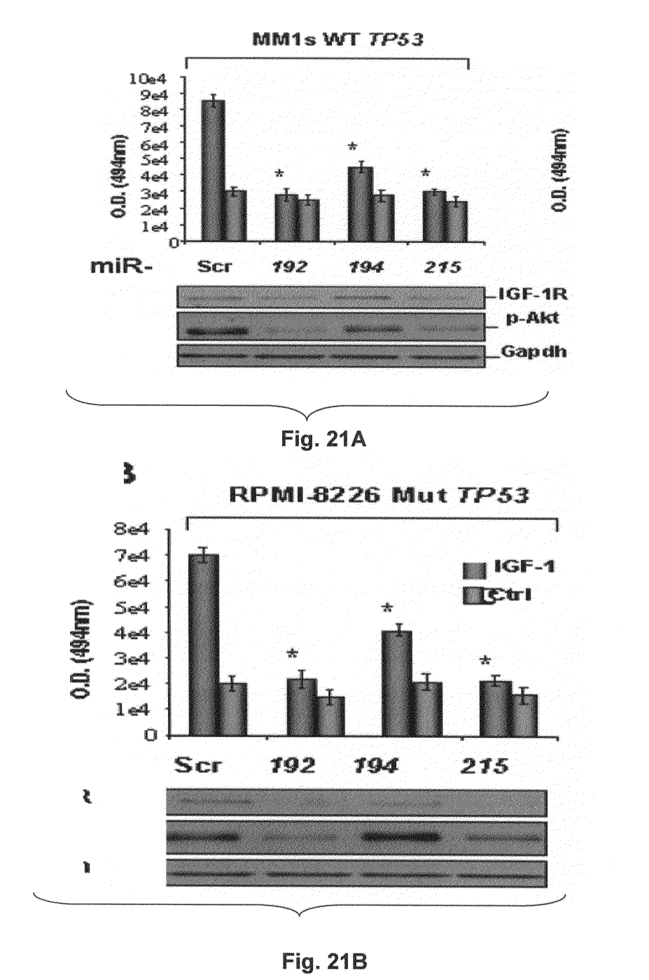

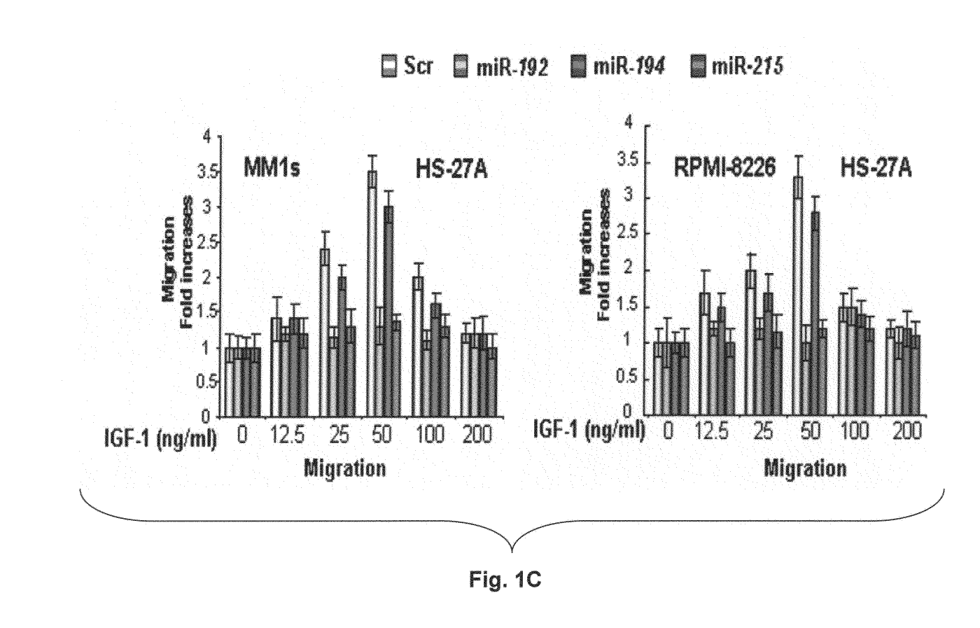

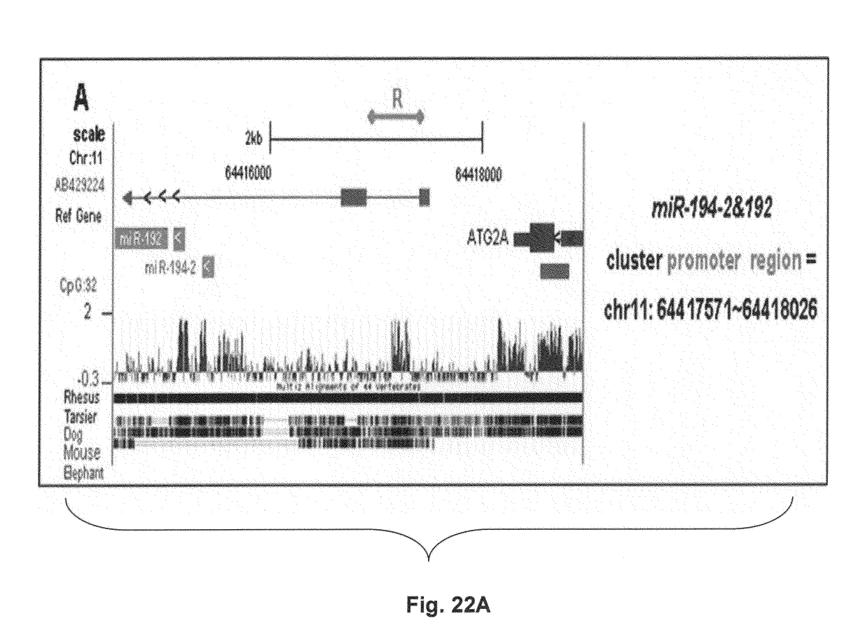

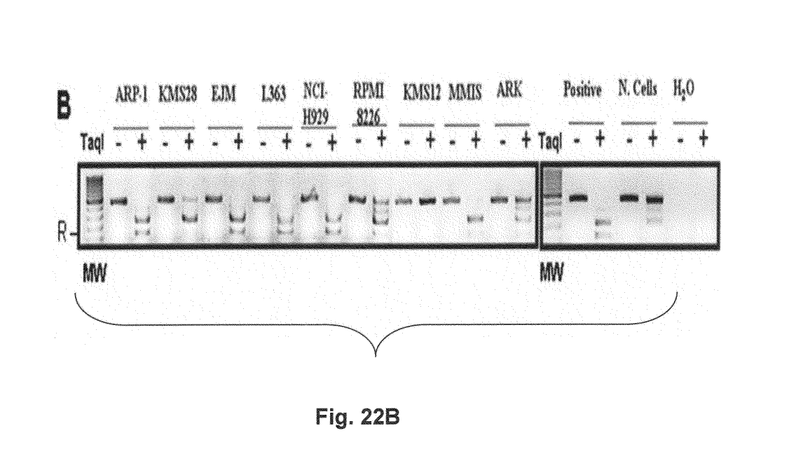

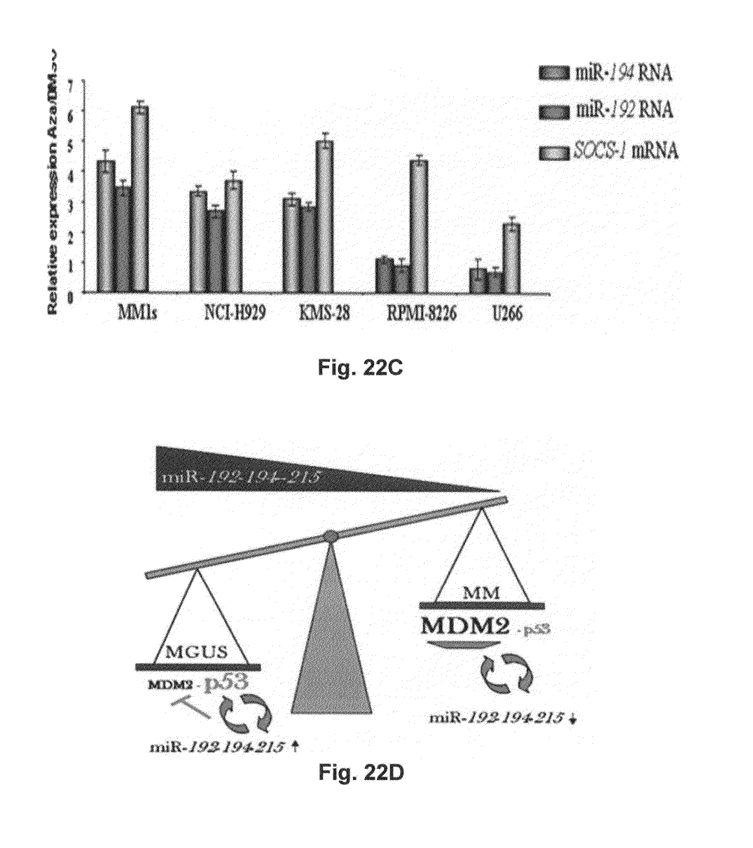

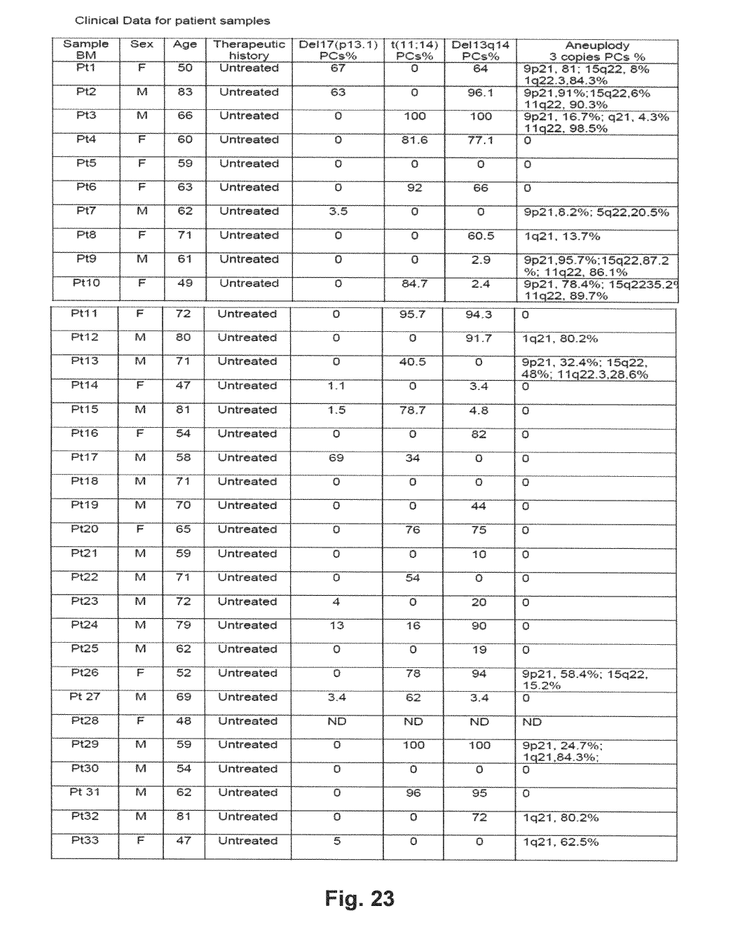

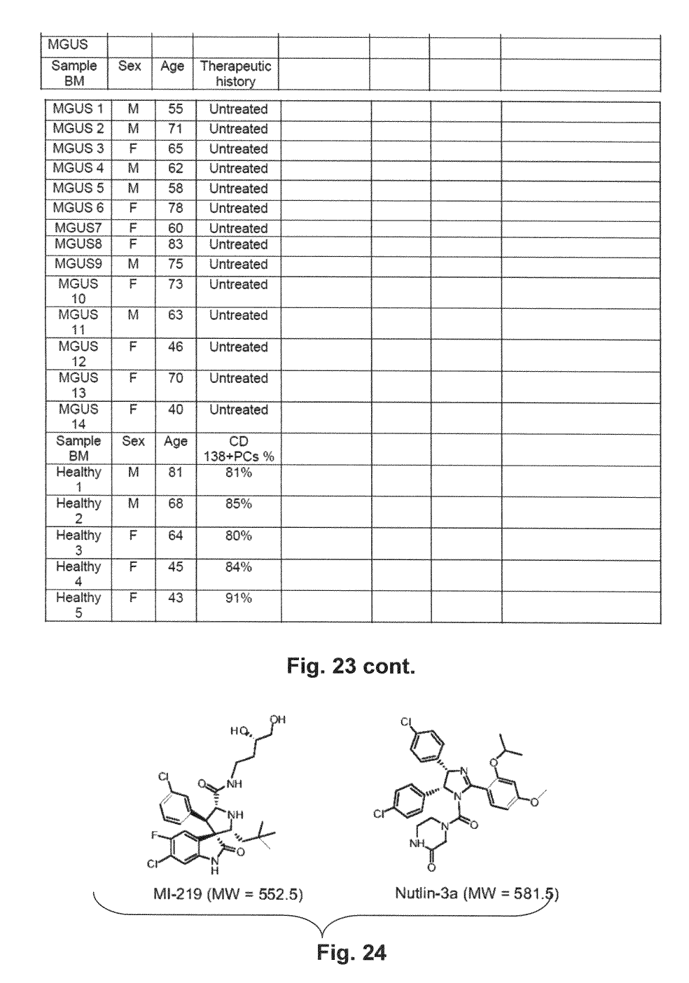

Increased expression of these miRs upon transfection was confirmed by qRT-PCR (FIG. 17A), and the effect on p53, MDM2 and p21 level was analyzed by Western blot in MM1s and NCI-H929 cells (FIG. 4A). Over-expression of miR-192 miR-194 and miR-215 significantly increased the level of p53 and p21 at 12 hr after Nutlin-3a treatment, compared to Scr-transfected cells (p<0.001), as shown by densitometric analysis in FIG. 4B, FIG. 4C. By contrast, expression of MDM2 protein was dramatically decreased in both cell lines (FIG. 4A, FIG. 4B, FIG. 4C).

Conversely, knockdown by 2′-O-me-anti-miR-192-194 and 215 (pool) after 12 hr of Nutlin-3a treatment, as confirmed by qRT-PCR (FIG. 17B) in TP53 WT cell lines, increased the level of MDM2 protein (p<0.01), while p21 and p53 protein levels were attenuated (p<0.01) (FIG. 4B), as confirmed by densitometry (FIG. 4E).

Furthermore, the inventors confirmed that MDM2 mRNA levels were strongly reduced in the miR-192, miR-194 and miR-215 transfected cells at 6 and 12 hr of Nutlin-3a treatment in both cell lines (FIG. 4C). These results show that these miRNAs induce the degradation of MDM2 mRNA, confirming that they regulate both protein and RNA level.

Next, the inventors tested whether MDM2 is a direct target of these miRNAs by performing a bioinformatics search (Target Scan), but were unable to identify the 3′-UTR of MDM2 as a target of miR-192, miR-194 and miR-215. Because the 3′-UTR of MDM2 is not well conserved across species, it was decided to use the RNA22 target prediction program that does not need validated targets for training, and neither requires nor relies on cross-species conservation. RNA22 predicted two miRNA responsive elements (MREs) for miR-192/215 and two MREs for miR-194 in the 3′UTR of human MDM2 (HDM2) (FIG. 4D and FIGS. 18A-18E).

To verify that HDM2 is a direct target of miR-192, miR-194 and miR-215, HDM2 3′UTR containing all MREs (˜4K), was cloned into pGL3 basic construct downstream of the luciferase open reading frame (FIG. 4D). This reporter construct was used to transfect the highly transfectable MM1s cells that express the endogenous miRs following up-modulated p53 expression. Increased expression of these miRs upon transfection significantly diminished luciferase expression (FIG. 4D).

The inventors subsequently screened the predicted MREs on 3′UTR of HDM2 mRNA using luciferase assays with 4 different constructs carrying the MREs for miR-192/215 and miR-194 (FIGS. 18F-18I). It was observed that expression of each specific MRE reporter construct was specifically down regulated upon transfection of each individual miRNA.

Conversely, when the inventors performed luciferase assays using a plasmid harboring the binding sites inactivated by site-directed mutagenesis, the inventors observed a consistent reduction in inhibitory effect (FIGS. 18F-18D. The inventors also analyzed the expression of MDM2 mRNA in a panel of CD 138+ PCs obtained from MM subjects (n.33), MGUS (n.4) subjects and normal donors (n.3) by RT-PCR (FIG. 4E). By Kruskal-Wallis analysis the inventors found that MDM2 mRNA is significantly up-regulated in MM samples (p<0.001) compared to MGUS samples and normal PCs (FIG. 4E). Furthermore, using non parametric test analysis, the inventors found a significant inverse correlation between miR-192 expression and MDM2 mRNA in MM samples (Sperman ρ=−0.698, p<0.0001, n=33) (FIG. 4F).

miR-192, miR-194 and miR-215 Re-Expression Enhances Sensitivity of WT TP53 MM Cells to Non Genotoxic Activation of p53 In Vitro and In Vivo

To determine if re-expression of the miRNAs could enhance sensitivity of WT TP53 MM cells to non genotoxic activation of p53, the inventors tested a new, highly selective, orally active small-molecule inhibitor of the MDM2-p53 interaction, MI-219. MI-219 is a spiro-oxindole composition as shown in FIG. 24, and described in Shangary, et al., PNAS, 105:3933-3938 (2008).

The inventors first examined whether MI-219 induces p53, MDM2, p21 and Puma up-regulation in MM1s cells after miR-192, miR-194 and miR-215 transfection. In cells with forced expression of miR-192 and miR-215, p53 became detectable even in untreated cells (FIG. 5A, FIG. 5B) (p<0.05). Dramatic p53 re-expression and consequent p21 and Puma up-regulation was observed in these cells and in miR-194 transfected cells, and is clearly visible following 24 hr of 2.5 μM MI-219 treatment (FIG. 5A), compared to control cells (Scr) where the treatment was ineffective (FIG. 5A). Densitometric analysis of p53 and MDM2 protein levels was performed when cells were treated for 24 hr with 2.5 μM, 5 μM and 10 μM MI-219 (FIG. 5B).

The inventors observed higher p53 accumulation (t2-fold increase, p<0.001) and dramatic MDM2 down-regulation (>2-fold decrease, p<0.001) in miRNA transfected cells (FIG. 5B). These opposing changes in MDM2 and p53 expression levels correlated with higher activation of p53 down-stream targets, p21 and Puma (FIG. 5A). Furthermore, MI-219 induced higher caspase-3 activation in presence of miR-192, miR-194 and miR-215 (p<0.001) (FIG. 5C).

Next, the inventors examined whether activation of p53 by MI-219 leads to apoptosis in MM cells. Indeed, treatment with MI-219 induced apoptosis as revealed by Annexin V staining (FIG. 5D). MI-219 effectively (p<0.0002) induced apoptosis in MM1s cells at 2.5 μM (27±3%) and 5 μM (32±3%) in cells transfected with a pool of miRNAs vs scrambled control. This effect was less significant when using 10 μM drug (30±5%), though it was enhanced when treatment was combined with miRNAs (55±5%) (FIG. 5D). Increased concentration of MI-219 did not increase the apoptotic rate of scrambled-transfected cells but caused non specific toxicity (data not shown).

The inventors investigated whether, in mouse xenograft models, the combined action of miRNAs and oral MI-219 would suppress tumorigenicity of MM cells. 8×10⁶ viable MM1s Gfp+/Luc+ cells were injected subcutaneously into the right flank of 40 nude mice. At 3 wk after injection a group of 32 mice with comparable tumor size were selected and randomly divided into 4 groups for 4 independent experiments, using 8 mice for each combined treatment (FIG. 5E).

Specifically, the inventors used the combination of oral treatment with 200 mg/kg MI-219 or vehicle control (VE) once a day for 14 days plus direct tumor injection of double strand RNA scrambled sequence (Scr) or a pool of premiR-192, -194 and -215 (miRs). Whereas the VE-Scr treated tumors increased 2-fold in volume in 2 wks (from 5390±993 mm³ to 13500±3200 mm³ [p<0.0001]), MI-219/Scr-treated tumors remained static in volume (5390±993 mm³ to 5400±1200 mm³) (FIG. 5E). In contrast, mice treated with VE-miRs showed ˜1.5-fold reduction in tumor size (from 5390±993 mm³ to 3700±950 mm³ [p<0.01]) and the most effective combination was MI-219 plus'miRs, where mice showed 5-fold reduced tumor volumes compared to tumors (from 5390±993 mm³ to 2100±560 mm³ [p<0.01]) and >93% reduction when compared to VE/Scr treatment (FIG. 5E). These findings demonstrate the usefulness of in vivo therapy of MM using combined miRNAs and MDM2 pharmacological inhibitor/s.

miR-192 and miR-215, by Antagonizing MDM2 Down-Regulation, Target IGF-1 and IGF1-R

Since this data demonstrate that miR-192, miR-194 and miR-215 target MDM2, the inventors sought to determine whether MDM2 substrates are also be affected. IGF-1R is a known target of MDM2 ubiquitin ligase function; therefore, by targeting MDM2, miR-192, miR-194 and miR-215 may indirectly influence expression of IGF-1R.

In MM cells, IGF-1R and its ligand, IGF-1, are key factors in regulation of PC migration into the bone marrow. The inventors noted that in WT TP53 MM cells, p53 re-expression was strongly associated with downregulation of IGF-1R and IGF-1 (FIG. 19A, FIG. 19B) compared to mutant TP53 MM cells (FIG. 19C, FIG. 19D).

Thus, the inventors sought to determine the effect of miRNAs on IGF-1R and IGF-1 expression through targeting MDM2. The inventors found that in the presence of miR-192, miR-215 but not miR-194, IGF-1R and IGF-1 protein levels decreased, as determined by Western blot analysis (FIG. 6A).

Furthermore, inhibition of endogenous miR-192/215 together, but not miR-194, using antisense oligonucleotides, increased both IGF-1R and IGF-1 levels in MM1s cells after 24 hr of Nutlin-3a treatment (FIG. 6B). To determine whether IGF-1 and IGF-1R would be mutually regulated in MM cells, the inventors silenced IGF-1 and observed up-regulation of IGF-1R protein level at 48 and 72 hr (FIG. 6C). This effect was clearly different from that observed following miRNA transfection (FIG. 6A). The inventors next tested whether miR-192 and miR-215 target IGF-1R and IGF-1 directly, by generating luciferase reporters containing their 3′ UTRs. Using Targetscan, Pictar and RNA22 searches the inventors identified several MREs for miR-192 and miR-215 but not for miR-194 in the 3′UTR of IGF-1R and IGF-1R mRNAs (FIG. 6D, FIG. 6E).

Luciferase activity dropped 40-50% when these constructs were co-transfected into MM1s cells with miR-192, 215 compared to miR-194 and Scr (FIG. 6D, FIG. 6E, FIG. 20A, FIG. 20B, FIG. 20C).

To determine if in freshly isolated CD-138+ PCs these miRNAs could regulate IGF-1 and IGF-1R expression, the inventors transfected miR-192, miR-215 (pool) into PCs of 9 subjects and at 48 hr the inventors performed immunofluorescence analysis using IGF-1R and IGF-1 antibodies. The inventors observed a significant decrease in IGF-1R and IGF-1 protein expression (FIG. 6F, FIG. 6G). Transfection efficiency was confirmed using RNA Fluorescent Oligo (FIG. 6H). These results indicate that miR-192 and miR-215 directly target IGF-1R and IGF-1 in MM.

miR-192 and miR-215 Block MM Migration and Invasion In Vitro and In Vivo

Given the role of IGF-1 and IGF-1R as anti-apoptotic factors and in MM migration through endothelial barriers and bone marrow stroma, the inventors determined whether miR-192 and miR-215 would interfere with the chemotactic function of IGF-1 and block migration and invasion of MM cells.

The inventors first determined that miR-192 and miR-215 actions on the IGF-1 axis in MM affect both WT TP53 (MM1s) and mutant (RPMI-8226) cell lines (FIG. 7A) and that the down-regulation of both proteins critically affects S6 and AKT phosphorylation in these cells.

Next, the inventors found that ectopic expression of miR-192 and miR-215 in NCI-H929 and RPMI-8226 IGF-1 treated cells was associated with significant decrease in cell adhesion (FIG. 21A, FIG. 21B), migration and tissue invasion compared to Scr control. To this end, the inventors used intra-epithelial trans-well migration assay with IGF-1 at various concentrations as attractant and two bone marrow-derived stromal cells, HS-5 (fibroblast-like) and HS-27A (epithelial-like) as cell layer.

As shown in FIG. 7B and FIG. 21C, IGF-1 (50 ng/ml) stimulated migration of MM cells, MM1s and RPMI-8226. To further examine the role of miRNA-192, miR-215 and miR-194 in MM cells, the inventors investigated the effect of these miRNAs on migration in vivo, using a homing model (Roccaro et al., Blood, 113:6669-6680 (2009)). Nine NOD-SCID mice (for each group) were intravenously injected with 8×10⁶ of pre-miRNA-192-, -194 and -215 or Scr probe-transfected GFP⁺/Lue⁺ MM1S cells. One wk later, mice were iv-injected every week for 4 wk with an individual miRNA or Scr dissolved in PBS (10 μg for each mouse). After 5 wk the homed and proliferated tumors were markedly suppressed in miRNA-treated mice compared to Scr-transfected MM cells (p<0.01) (FIG. 7C).

At 5 wk post-injection the inventors first noted reduced tumor progression, by bioluminescence imaging (FIG. 7D). Mice injected with Scr-transfected MM1s plus serial iv-injection with Scr showed significant tumor growth, but tumor burden was significantly reduced in mice injected with pre-miR-194 and was nearly nonexistent in mice injected with either pre-miR 192 or pre-miR-215 (FIG. 7C, FIG. 7D).

Secondly, through analysis of bone marrow engraftment of these cells in injected NOD-SCUD mice by FACS analysis using human CD-138+ antibody, the inventors confirmed that Scr-treated mice showed bone marrow engraftment of ˜25±15% of MM1s vs 4±2% for miR-192 and 2±2% for miR-215-transfected (FIG. 7E). The in vivo action of miR-194 was less effective, 12±3% compared to miR-192 and miR-215 but still effective when compared to control (FIG. 7E).

These data show that miR-192, miR-215 and miR-194 have therapeutic utility, not only by affecting proliferation rate in MM cells, but also by affecting the homing and migration ability of MM cells.

Discussion of Examples

Complex cytogenetic abnormalities and numeric chromosomal aberrations occur in virtually all MMs, and in most, if not all, cases of MGUS. Paradoxically, mutations and/or deletion of TP53 occur in only a small percentage of intramedullary MMs, and not at all in MGUS.

The data (see, for example, FIG. 4E) show that MDM2 over-expression, not associated with MDM2 gene amplification, in MMs is, at least in part, responsible for p53 inactivation in cells retaining functional p53 pathways; and, that induction of p53 is useful for treatment of MM.

The inventors show, for the first time herein, the role of miRNAs in the p53 apoptotic pathway upon non genotoxic activation of p53 in MM cells using small molecular inhibitors of MDM2 (Nutlin-3a, MI-219). The increased expression of two related microRNA clusters located in regions considered important for MM (miR-194-2-192 at 11q13.1 and miR-194-1-215 at 1841.1) upon p53 activation in MM cells is also described herein.

These miRNAs are direct p53 targets, through characterization of the miR-194-2-192 cluster promoter region and definition of a new p53 consensus site. In subject samples, the expression of these miRNAs changed during transition from normal PC, via MGUS to intramedullary MM. In addition, these miRNAs were significantly down-regulated in a cohort of newly diagnosed MMs vs MGUS; miR-192, miR-215 and miR-194 enhanced colony suppression, cell cycle arrest or apoptosis in a p53-dependent manner. Further, their biological action could be associated to MDM2 status in MM cells (for example, the case of KMS28BM). The short half-life of MDM2 protein and attendant difficulties in analyzing its protein expression in MM cells without p53 activation, led to the inventors demonstration that the effect of these miRNAs on MDM2 was clearly detectable after treating WT TP53 MM cells with combined Nutlin-3a and ectopic expression of the miRNAs.

In treated cells with enforced expression of these miRNAs, MDM2 was dramatically down-regulated at protein and mRNA levels and this down-regulation was inversely associated with higher p53 expression and p21 activation (FIG. 4A, FIG. 4B, FIG. 4C).

Luciferase assays using plasmids harboring MDM2 3-UTR sequence strongly confirmed that MDM2 is the direct target of these miRNAs. In a subset of newly diagnosed MMs, elevated levels of MDM2 mRNA were inversely associated with miR-192 expression.

The inventors now show herein in vivo and in vitro that the combination of miRNAs with p53 pharmacological activator (e.g., MI-219), leading to MDM2 down-regulation and p53, p21, Puma up-regulation, is a successful therapeutic strategy, producing anti-tumor results that could not be achieved solely by increasing drug concentration.

The inventors also found that miR-192 and miR-215 expression, by overriding MDM2 ubiquitination of IGF-1R, directly targets the IGF-1 axis in MM cells, controlling mobility and invasive properties of MM cells in vitro and in vivo.

FIG. 22D is a model which shows that these miRNAs are regulators of the auto-regulatory loop, increasing the window of time between p53 apoptotic action and p53 degradation by MDM2. A; and are, at the same time, targeting the IGF axis, antagonizing MDM2 ubiquitin ligase function on IGF-1R (see FIG. 8).

During Nutlin-3a treatment of primary CD-138+ PCs from MMs without TP53 deletion, the inventors noted that p21 activation, as well as re-expression of the three miRNAs, was consistent but not uniform in all samples analyzed.

These miRNA genes are located in chromosome regions in MM that are normally characterized by chromosome gain and translocations rather than deletions; thus, gene deletion does not seem to be the answer. Then, the inventors determined the methylation status in the promoter of the miR-194-2-192 cluster. By combined bisulfite restriction analysis (COBRA) the inventors detected hypermethylation of the promoter region of this cluster (Region R) (FIG. 22A) in MM cell lines (FIG. 22B). Furthermore, treatment of MM cell lines with a demethylation agent (Azacytidine) increased the expression of these miRNAs in WT TP53 MM cell lines (FIG. 22C).

Re-expression of the SOCS-1 gene, known to be silenced by hypermethylation in MM cells, served as internal control. While not wishing to be bound by theory, the inventors herein now believe that the transition from MGUS to MM is favored by clonal selection of cells with aberrant promoter methylation of this miR cluster, in association with decreasing ability of p53 to down-modulate MDM2 expression due to decreased expression of miR-194 and 192, thus tipping the regulatory balance in favor of MDM2 in MM cells.

Of note, monoallelic deletion of TP53 in MM, which often seems to occur without mutation on the other allele, is associated with an extremely poor prognosis. A two-fold decrease in TP53 gene content is associated with tumor progression, which supports the inventors' belief that a partial lack of expression of these miRNAs in MMs could create a p53 imbalance with direct biological consequences.

Further, the inventors herein have now have defined a new mechanism of p53 regulation through miRNAs acting on MDM2 expression. These miRNAs are useful for therapeutic targeting, as illustrated in FIG. 8. Furthermore, since these miRNAs can act at several levels as tumor suppressors, the results provide the basis for the development of new miRNA-targeted therapies for MM.

Experimental Procedures

Collection of Primary Cells

33 newly diagnosed primary MM samples, 14 primary samples from monoclonal gammopathy of undetermined significance (MGUS), and 5 primary samples from healthy donors were obtained from bone marrow aspirates. Written informed consent was obtained in keeping with institutional policies (IRB-approved procurement protocol (2000C0247) at The Ohio State University and University of Turin GIMEMA-MM-03-05, N. EUDRACT 2005-004745-33). Three of the five healthy PCs used were purchased from AlCells LLC (Emeryville, Calif.).

### Luciferase Assays

MM1s cells were cotransfected with 1 μg of pGL3 firefly luciferase reporter vector, 0.1 μg of the phRL-SV40 control vector (Promega), and 100 nM miRNA precursors (Ambion) using nucleoporation (LONZA) Cell Line Nucleofector Kit V. Firefly and Renilla luciferase activities were measured consecutively by using the Dual Luciferase Assay (Promega) 24 hr after transfection. Each reporter plasmid was transfected at least twice (on different days) and each sample was assayed in triplicate.

CD-138+ PCs Purification, MM cell lines collection and Rrowth conditions

Plasma cells, CD 138 cells were purified from total marrow cells of subjects by Human Whole Blood CD138+ Selection Kit (Cat#18387, Stem Cell Technologies) as per the manufacturer's instructions. Yield and purity of CD138+ cells was evaluated by flow cytometry using anti-CD138 antibody (Becton Dickinson). Primary cells that were used for in vitro experiments were cultured in RPMI-1640 (Sigma) supplemented with 15% fetal calf serum and kept in culture for 24 h before specific treatment. MM cell lines (MM1s, NCI-H929, KMS28, RPMI-8226, U266 and JJN3) [courtesy of Dr M. Kuehl (National Cancer Institute, MD) were cultured in RPMI-8226 (Sigma) and 10% fetal bovine serum (Cat#019K8420, Sigma). Human bone marrow stromal cell lines HS-27A and HS-5 were purchased from American Type Culture Collection (Chantilly, Va.) and cultured in RPMI 1640 containing heat-inactivated 5% fetal bovine serum (FBS).

Transfection Method for Primary Cells and MM Cell Lines.

CD-138+ PCs obtained from new diagnosed MM subjects and isolated as previously described were transfected by using nucleoporation (LONZA) Cell Line Nucleofector Kit V (Cat#VCA-1003). MM1s, NCI-H929 cell lines were transfected by using nucleoporation (LONZA) Cell Line Nucleofector Kit V (Cat#VCA-1003). Instead for U266, KMS-28BM and RPMI-8226 Cell Line Nucleofector Kit C (Cat#VCA-1004) was used. Specifically for primary cells 1×10⁶ CD-138+ PCs were resuspended in 100 μA of V solution and 100 nM miRNA precursors (Ambion) was used for the transfection reaction. For MM cell lines 5×10⁶ cells were re-suspended in 100 μl of nucleofector solution V/C and 100 nM of miRNAs precursor or 100 nM LNA miRNA antisense oligonucleotides (Ambion) was used for each transfection point. Protein lysates and total RNA were collected at the time indicated. miRNA processing and expression were verified by northern blot and stem-loop qRT-PCR.

The inventors confirmed transfection efficiency using BLOCK-IT Fluorescent Oligo (Invitrogen) for all the cell lines. Untreated cells transfected with negative control oligonucleotides were used as a calibrator.

RNA-DNA-Protein Extraction from Primary Cells

Total RNA-DNA-Protein from primary CD-138+ PCs was extracted using RNA/DNA/Protein purification kit from NORGEN (cat#23500, Thorold, ON, Canada) following the manufacture's instructions. Briefly 350 μl of lysis solution was added to 1×10⁶ CD-138+ PCs pellet. The cells were lysed by vortexing and 200 μl of 95% ethanol was added to the lysate. The entire lysate volume was loaded to the provided columns. After several column washes the RNA was eluted using 35 μl of RNA Elution Solution. The same column was then washed with 500 μl of gDNA and the genomic DNA using 40 μl of gDNA Elution buffer. For the protein extraction the flowthrough from the RNA binding step was applied following the manufacture's instructions onto the provided column, washed and eluted using 100 μl of the provided buffer.

RNA Extraction from MM Cell Lines.

Total RNA from MM cell lines (RPMI-8226, U266; JJN3; NCI-H929; MM1s; KM28BM) was extracted using TRIzol Reagent Invitrogen (Cat#15596-018) following the manufacture's instruction. Specifically the pellet obtained from 5×106 cells was lysed 1 ml of TRIzol solution. At the end of the extraction the isolated RNA was dissolved in 35 μl in RNase-free water and incubated for 10 min at 55 C.

Microarray Experiments.

The total RNA from MM cells used for microarray analysis was isolated with TRiz extraction reagent (Invitrogen). miRNA microchip experiments were performed. The miRNA microarray was based on a one-channel system. Five micrograms of total RNA was used for hybridization on the OSU custom miRNA microarray chips (OSU_CCC version 3.0), which contains N1,100 miRNA probes, including 345 human and 249 mouse miRNA genes, spotted in duplicates. The data were analyzed by microarray images by using GenePix Pro 6.0. Average value of the replicate spots of each miRNA was background-subtracted and subjected to further analysis. MiRNAs were retained when present in at least 50% of samples and when at least 50% of the miRNA had fold change of >1.5 from the gene median.

Data Analysis for microarray experiments.

Microarray images were analyzed by using GenePix Pro 6.0. Average values of the replicate spots of each miRNA were background-subtracted and subject to further analysis. MiRNAs were retained when present in at least 50% of samples and when at least 50% of the miRNA had fold change of more than 1.5 from the gene median. Absent calls were thresholded to 4.5 in log 2 scale before normalization and statistical analysis. This level is the average minimum intensity level detected above background in miRNA chips experiments. Quantiles normalization was implemented using the Bioconductor package/function. Differentially expressed microRNAs were identified by using the univariate t test within the BRB tools version 3.5.0 set with a significant univariately at alpha level equal to 0.01. This tool is designed to analyze data using the parametric test t/F tests, and random variance t/F tests. The criteria for inclusion of a gene in the gene list is either p-value less than a specified threshold value, or specified limits on the number of false discoveries or proportion of false discoveries. The latter are controlled by use of multivariate permutation test.

q-RT-PCR.

The single tube TaqMan miRNA assays from Applied Biosystems (miR-192 #000491; miR-215 #000518; miR-194 #000493; miR-34a #000425; miR-15a #000389; miR-29a #002112; miR-29b #000413) were used to detect and quantify mature miRNAs using ABI Prism 7900HT sequence detection systems (Applied Biosystems). Normalization was performed with RNU44 (Applied Biosystems Assay #00194) or RNU48 (Applied Biosystems Assay #001006). Comparative real-time PCR was performed in triplicate, including no-template controls. Relative expression was calculated using the comparative Ct method.

Western Blot Analysis.

Samples were extracted in 15 mM Tris.Cl, pH 7.5/120 mM NaCl/25 mM KCl/2 mM EGTA/0.1 mM DTT/0.5% Triton X-100/10 mg/ml leupeptin/0.5 mM PMSF. Total protein (35 μg) from each sample was separated on a 4-20% Tris-HCl-Criterion precast gel Bio-Rad (cat#345-0032, Hercules, Calif.) and transferred to a poly(vinylidene difluoride) filter (Millipore). The filter was blocked in 5% nonfat dry milk, incubated with the specific antibody, washed, and probed with secondary antibody IgG conjugated to horseradish peroxidase (Santa Cruz Biotechnology), and developed with enhanced chemiluminescence (Amersham Pharmacia). Immunoblot analyses were performed using the following antibodies: p53 (sc-53394, Santa Cruz Biotechnology), MDM2 (sc-965, Santa Cruz Biotechnology), phospho-MDM2 (Cat#3521, Cell Signaling), c-MYC (cs-40, Santa Cruz Biotechnology), IGF-1 (sc-9013, Santa Cruz Biotechnology), IGF-1R (Cat#3027, Cell Signaling), total-Akt (Cat#9272, Cell signaling), phospho-Akt (Cat#4060, Cell Signaling), total-S6 (Cat#2217, Cell Signaling), phospho-S6 (Cat#2211, Cell Signaling), p21 (sc-817, Santa Cruz Biotechnology), α-PUMA (Cat#4976, Cell Signaling), GAPDH (Cat#2118, Cell Signaling). Filters were reprobed with enzyme-conjugated antibodies to GFP and β-actin (Santa Cruz Biotechnology).

Nutlin3a and MI-219 Treatment.

MM cells (from MM subjects or from cell lines) non-transfected or transfected with pre- or ASOs miR-192, miR-215, miR-194 and Scr sequence as described above, were treated with MDM2 inhibitor (Nutlin3a and MI-219). Fresh CD138+ primary PCs isolated from new diagnosed MM subjects as previously described were maintained in culture for 24 hr and then treated for 24 hr with 10 μM Nutlin-3a (Cayman Chemical Company) or vehicle (DMSO). For transfected cells at 24 hrs after transfection MM cells were treated with 10 μM Nutlin-3a (Cayman Chemical Company) or DMSO vehicle only at different time points. MMIS cells were also treated with MI-219 solution (10% PEG400/3% Cremophor EL/87% 1×PBS) or only vehicle (10% PEG400/3% Cremophor EL/87% 1×PBS) at different concentration (2.5, 5 and 10 μM) for 24 hr and collected for RNA and protein extractions.

RT-PCR

RNA was isolated from cell lines using Trizol reagent (Invitrogen) as per the manufacturer's protocol. An aliquot of 5 μg RNA was then used for cDNA synthesis using the SuperScript first strand cDNA synthesis kit (Invitrogen). RT-PCRs were carried out using ABI Prism 7900HT sequence detection systems with Applied Biosystems TaqMan Gene expression assays (p21 (CDKN1A):Hs01121172_ml; MYC:Hs99999003_ml; TP53:Hs00153349_ml; MDM2: Hs01066938_ml).

Northern Blotting.

Total RNA was extracted with TRIzol solution (Invitrogen) and the integrity of RNA was assessed with an Agilent BioAnalizer 2100 (Agilent, Palo Alto, Calif., USA). Northern blotting was performed. The oligonucleotides used as probes were the complementary sequences of the mature miRNA (miRNA registry).

Cell Viability Assay and Apoptosis Assay.

Cells were plated in 96-well plates in triplicate and incubated at 37° C. in a 5% CO₂ incubator. Cell viability was examined with 3-(4,5-dimethylthiazol-2-yl)-2,5-dipheniltetrazolium bromide (MTT)-Cell Titer 96AQueous One Solution Cell Proliferation Assay (Promega), according to the manufacturer's protocol. Metabolically active cells were detected by adding 20 μl of MTT to each well. After 1 hr incubation, the plates were analyzed in a Multi label Counter (Bio-Rad Laboratories). Apoptosis was assessed using Annexin V-FITC apoptosis detection kits followed by flow cytometric analysis. For Annexin V staining, MM cells (pre-miRNAs pool or Scrambled transfected) were treated with MI-219 at different concentrations (0, 2.5, 5.0, 10 μM) for 24 hr and then treated as for DNA content analysis, except that fixation was omitted and the Cells (5×10⁵ per sample) were resuspended in PBS containing 25 μg/ml Annexin-V-FLUOS (Roche Applied Science) and 50 μg/ml PI prior to FACS analysis. The percentage of apoptosis indicated was corrected for background levels found in the corresponding untreated controls. The percentage of apoptotic cells was expressed as the mean±SD of three experiments.

Colony Assay.

A total of 30×10³ cells were infected with Lenti-mir-192: PMIRH 192-PA-1 Lenti-mir-194-1:PMIRH 1941PA-1; Lenti-mir-215:PMIRH 215 PA-1; Control Lentivector(pCDH-CMV-EFI-copGFP cDNA cloning and Expression vector): CD511 B-1 (System Biosciences) as per the manufacturer's protocol. MM cells were plated in quadruplicate in 1 mL of methylcellulose medium for Mouse cells (Cat#03234, Stem cell Technologies) in 6-well culture plates. Colonies consisting of more than 40 (125μ) cells were scored at 14 days.

Cell Cycle Analysis.

Nocodazole was (Sigma-Aldrich) was dissolved in DMSO as a stock solution of 10 mg/ml for cell cycle arrest in G2/M phase. Cells were first arrested and synchronized in G2/M phase by growth in 80 nM nocodazole for 16 hr. Cells were then washed and fresh medium added. After 6 hr, cell cycle analysis was performed by propidium iodide staining. Corresponding amounts of DMSO alone were added in control experiments. In experiments involving transfection and MI-219 treatment, the cells were first transfected, incubated for 24 hr, and then treated with the chemotherapeutic drug for 24 hr. For DNA content analysis, cells were fixed in methanol at −20° C., washed again, rehydrated, re-suspended in PBS containing 50 μg/ml propidium iodide (PI) and 50 μg/ml RNase A, and analyzed by flow cytometry (Becton Dickinson). For detection of caspase 3 activity, KMS28BM and MM1s cells were cultured in 96-well plates and treated with Nutlin-3a. After the treatment the cells were analyzed using Caspase-Glo 3 Assay kit (Promega) according to the manufacturer's instructions. Continuous variables were expressed as mean values ± standard deviation (s.d.).

Adhesion Assay.

Before adhesion, MM cell lines were starved overnight in RPMI 1640/0.5% BSA, without loss of viability. Cells (5×10⁶/ml) were labeled with calcein-a.m. (Molecular Probes, Eugene, Oreg.) for 30 min at 37° C., washed, and resuspended in adhesion medium (RPMI 1640/10% FBS). Cells were stimulated with or without IGF-1 at 0-200 ng/ml for 20 min and added in triplicate to Fibronectin-coated 48 well plates (BD Biosciences #354506, Bedford, Mass.) at 37° C. for 30 min, and unbound cells were removed by four washes with RPMI1640. The absorbance of each well was measured using 492/520 nm filter set with a fluorescence plate reader (Wallac VICTOR2; Perkin-Elmer, Boston, Mass.).

Transendothelial Migration Assay.

IGF-I-induced MM transendothelial migration was determined using 24 well, 6.5 mm internal diameter transwell cluster plates with polycarbonate membranes (5 μM pore size) separating the 2 chambers (Corning Costar, Cambridge, Mass.). Bone marrow stromal cell lines HS-5 and HS-27A were grown on the insert for 24 hrs to produce a confluent monolayer. IGF-I or SDI-1\dot{α} diluted to varying concentrations in RPMI 1640 was loaded in the lower chamber. MM cell suspensions starved for 3 hrs in serum-free RPMI 1640 were loaded onto the insert (upper chamber). Plates were then incubated for 4 hr at 37° C. At the end of the incubation period, cells migrating through endothelial or bone marrow stromal cell layers into the lower chamber were harvested, stained with trypan blue, and counted under a microscope.

Chromatin Immunoprecipitation Assay.

Chromatin immunoprecipitation was performed as described by de Belle et al., Biotechniques, 29, 162-196, 2000, with slight modifications. Cells (5×10⁶) from MM1s treated with Nutlin-3a were fixed in 1% formaldehyde for 10 min at 37° C. for chromatin cross-link. Cells were washed with ice-cold 1×PBS, scraped in 1×PBS plus protease inhibitors, and collected by centrifugation. Cell pellets, resuspended in cell lysis buffer [50 mmol/L Tris-HCl (pH 8.0), 10 mmol/L EDTA, and 1% SDS] plus protease inhibitors. The probes were sonicated 25× for 30 s with a Bioruptor sonicator (Diagenode) and pelleted. The supernatant was diluted with dilution buffer 117 mmol/L Iris (pH 8.0), 167 mmol/L NaCl, 1.2 mmol/L EDTA, 1.1% (v/v) Triton X-100, 0.01% (w/v) SDS]. DNA-protein complexes were immunoprecipitated using 5 μg of the anti-p53 antibody (Santa Cruz) or with mouse polyclonal IgG control (Zymed). Cross-links in the immunoprecipitated chromatin were reversed by heating with proteinase K at 65° C. overnight, and DNA was purified by the MinElute Reaction Cleanup column (Qiagen) and resuspended in water. The purified chromatin was subjected to PCR and the products were analyzed by gel electrophoresis using 2% agarose. The following primers were used:

| p53 binding site in miR-194-2-192 | |
|---|---|
| cluster promoter | |
| [SEQ ID NO: 1] | |
| For: | 5′-TGGGTGGGTCCATGGGGAAAC-3′; |
| [SEQ ID NO: 2] | |
| Rev: | 5′-GCTTCTGCTCTGTTC CCAGT-3′. |
| Negative control for miR-194-2-192 | |
| cluster promoter region: | |
| [SEQ ID NO: 3] | |
| For: | 5′-AGGCCCTGGAGGAGAC AG-3′; |
| [SEQ ID NO: 4] | |
| Rev: | 5′-CAGGGGTCCTACCACTCAGG-3′. |
| miR-34a promoter (positive Control): | |
| [SEQ ID NO: 5] | |
| For: | 5′-ACGCTTGTGTTTCTCAGTCCG -3′; |
| [SEQ ID NO: 6] | |
| Rev: | 5′- TGGTCTAGTTCCCGCCTCCT -3′. |
| miR-215-194-1 cluster promoter: | |
| [SEQ ID NO: 7] | |
| For: | 5′-AGCAGGCTUTGGCTCTGATT-3′; |
| [SEQ ID NO: 8] | |
| Rev: | 5′-CCAGCCTCTTCTAT GCCAGA-3′; |
| CDKN1A promoter (positive control): | |
| [SEQ ID NO: 9] | |
| For: | 5′-TTGTTCAATGTATCCAAAAGAAACA-3′; |
| [SEQ ID NO: 10] | |
| Rev: | 5′-TGAGATAAAGCTTCTTCCCTTAAAAA3′; |
| hMT-RNR2 promoter (negative control): | |
| [SEQ ID NO: 11] | |
| For: | 5′-CATAAGCCTGCGTCAGATCA-3′; |
| [SEQ ID NO: 12] | |
| Rev: | 5′-CCTGTGTTGGGTTGACAGTG-3′. |

Immunofluorescence

The effect of miRNAs pool (miR-192, miR-215) or scrambled sequence on MM samples (n=9) was assessed by immunocytochemical method. At 24 hr after transfection cells were attached to the slide by cytospin technique. Briefly, cells were fixed and permeabilized by incubation in ice-cold acetone and the washed in PBS. Cells were incubated for 1 hr with 5% BSA and then incubated over-night with 1:100 dilution in PBS of IGF-1R and IGF-1 antiserum (Santa Cruz Biotechnology; Cell Signaling) and then incubated with Alexa Fluor 488 donkey anti-rabbit IgG (Molecular Probes). The slides were mounted in mounting medium for fluorescence with DAPI (Vector, Burlingane, Calif.) and visualized using an epifluorescence microscope (Nikon Eclipse E800; Nikon, Avon, Mass.) and a Photometrics Coolsnap CF color camera (Nikon, Lewisville, Tex.), as previously described.

Statistical Analysis.

Student's t test and one-way analysis of variance was used to determine significance. All error bars represent the standard error of the mean. Statistical significance for all the tests, assessed by calculating p value, was <0.05. Sperman correlation coefficient was calculated to test the association between miR-192 and MDM2 mRNA in MM samples (n=33). Expression values (obtained by qRT-PCR) from the 4 healthy PCs, 14 MGUS and 33 MM samples for each of the 3 miRNAs (miR-192, miR-215 and miR-194) were tested using the Bartlett test to evaluate the homogeneity of the variance among the samples. Kruskal-Wallis was used to assess whether the 3 miRNAs are differentially expressed among normal PCs, MGUS and MM samples on the basis of the Bartlett test P value. The Kruskal-Wallis test was used for Bartlett test P values less than 0.001.

Target Screening.

Three publicly available search engines were used for target prediction to obtain the putative targets: TargetScan (Release 2.1); genes.mit.edu/targetscan, Pictar; pictar.bio.nyu.edu and Rna22; and cbcsrv.watson.ibm.com/rna22_targets.html. For RNA22 predicted sites the inventors considered only the heteroduplex with a folding energy >−27 Kcal/mol (FIG. 18B, FIG. 18C, FIG. 18D, FIG. 18E) because the inventors were not able to confirm by luciferase assay the interactions between target gene and miRNAs with a folding energy less that −27 Kcal/mol (data not shown).

Detection of Tumor Progression by Bioluminescence Imaging.

Mice were injected with 75 mg/kg of Luciferin (Xenogen, Hopkington, Mass.), and tumor growth was detected by bioluminescence 10 min after the injection. The home-built bioluminescence system used an electron multiplying CCD (Andor Technology Limited, Belfast, United Kingdom) with an exposure time of 30 sec, and an electron multiplication gain of 500 voltage gain×200, 5-by-5 binning, and with background subtraction. Images were analyzed using Image-J software (National Institutes of Health, Bethesda, Md.).

In Vivo Experiments.

Animal studies were performed according to institutional guidelines. For the sub-cutaneous engraftment model 8 wk old male athymic nu/nu mice (Charles River Laboratories, Wilmington, Mass.) were maintained in accordance with IACUC procedures and guidelines. 8×10⁶ of GFP/Luc+MM1s cells were suspended in 0.10 ml of extracellular matrix gel (BD Biosciences) and the mixture was injected subcutaneously into the right flank. 3 wks after injection, mice with comparable size tumors, as detected by bioluminescence images, were treated for 2 wks with a combination of oral dose of MI-219 (200 mg/kg) or vehicle, once a day for 14 days and miRNAs or scrambled sequence oligos (10 ug) (Ambion), injected directly into the tumors once a week for 2 wks. Measurements of xenograft growth were performed, and tumor volume was estimated using the formula 4/3 a (L*W*H/8). Tumor size was assessed by digital caliper. For the NOD-SCUD engraftment model Luc+/GFP+MM.1S cells (pre-miR-192, 194, 215 or Scr-transfected, as described above) (8×10⁶/mouse) were injected into the tail vein of SCUD mice. Treatment started 7 days from tumor cell inoculation, by weekly i.v. injections of miRNAs or scrambled sequence. RNA oligos (Ambion) (10 μg) for four cycles (4 wks total). Tumor size was assessed every 7 days by, bioluminescence images. Thirty-five days after injection, mice were analyzed by bioluminescence images and then sacrificed. MM1s bone marrow isolated cells were stained with anti-human CD-138 antibody (BD) and analyzed by FACS analysis. Statistical significance of differences between control and treated animals was evaluated using Student's t test. Animal experiments were conducted after approval of the Institutional animal care and use committee, Ohio State University.

Combined Bisulfate Restriction Analysis (COBRA).

COBRA analysis was performed largely as described in Xiong et al., Nucleic Acids Res. 25:2532-2534 (1997). A sample of 1 μg of genomic DNA was modified with sodium bisulfite using the CpGenome modification kit (Intergen, Oxford, UK) as per the manufacturer's instructions. PCR products were digested with a restriction enzyme specific for the methylated sequence after sodium bisulfite modification. For the CpG island primers, digestion of the total PCR products was carried out with 20 U BsiEI (New England Biolabs, Hitchin, UK) in 1× manufacturer's buffer supplemented with 100 μg/ml bovine serum albumin for 2 hr at 60° C. For the promoter primers, digestion of the total PCR products was carried out with 20 U TaqI (Invitrogen, Paisley, UK) for the region R1. Digested PCR products were separated on 2% agarose gels and visualized by ethidium bromide staining on GelDoc 1000 (Bio-Rad, Hemel Hempstead, UK). The primers used for the PCRs (and positions relative to the transcriptional start site) were:

| for the CpG island, miR-192 | |
|---|---|
| Region1 | |
| [SEQ ID NO: 13] | |
| For: | 5′-GGGTATTGGGAATAGAGTAGAA-3′; |
| [SEQ ID NO: 14] | |
| Reverse: | 5′-CACCCTTCAAAAAAATACCTA-3′. |

Luciferase Reporter Vector.

HDM-2, IGF-1R, IGF-1 3′UTR containing predicted microRNA binding site were amplified by PCR from genomic DNA (293T/17cells) using AccuPrime Taq DNA (Cat no. 12346-086, Invitrogen, Carlsbad, Calif.) and inserted into pGL3 control vector (Promega) by using XbaI site immediately downstream from the stop codon of firefly luciferase. Deletion of the first six nucleotides of each complementary seed-region complementary site were inserted in mutant construct using quick change site directed mutagenesis kit from Stratagene (Cat#200517-5, Cedar Creek, Tex.), according to the manufacture's protocol. The primers sequences are listed herein.

In case of promoter assay, miR-194-2-192 cluster promoter were amplified by PCR from genomic DNA (293T/17cells) and cloned into pGL3 basic vector (Invitrogen) by using SacI-XhoI sites. To obtain miR-192-2-192 cluster promoter constructs with point mutations in p53 binding site directed mutagenesis kit from Stratagene (Cedar Creek, Tex.) was used (primers listed below).

List of primers used for Luciferase reporter vectors.

MDM2 3′ UTR primers:
MRE (2117-38) for miR-194
For:
[SEQ ID NO: 15]
5′-ATTTCTAGAAATTCTTGGCTGGACATGGT-3
Rev:
[SEQ ID NO: 16]
5′-ATTTCTAGATCAAAGTGAGAAAATGCCTCAA-3′
MRE (3495-4497) for miR-192/215:
For:
[SEQ ID NO: 17]
5′-ATTTCTAGATTCCCAGCCTAGGTTTCAGA-3′
Rev:
[SEQ ID NO: 18]
5′-ATTTCTAGATGAGATGCGATCAAACATCC-3′
MRE (5974-95) for miR-194:
For:
[SEQ ID NO: 19]
5′-ATTTCTAGACAATAAATGGCCAAAGGGATT-3′
Rev:
[SEQ ID NO: 20]
5′-ATTTCTAGACTTCAAGCTGCCCAGTGATA-3′
MRE (6360-80) for miR-192/215
For:
[SEQ ID NO: 21]
5′-ATT TCTAGACAATAAATGGCCAAAGGGATT-3′
Rev:
[SEQ ID NO: 22]
5′-ATT TCTAGACAAAAGCTAGTCCCCGTCTG-3′
Full (2117-6380):
For:
[SEQ ID NO: 23]
5′-ATTTCTAGAAATTCTTGGCTGGACATGGT-3′
Rev:
[SEQ ID NO: 24]
5′-ATT TCTAGACAAAAGCTAGTCCCCGTCTG-3′
Deletion primers for MDM2 3′-UTR
upon request.
IGF1 3′UTR primers: MRE for miR-192/215
F:
[SEQ ID NO: 25]
5′-ATTTCTAGAGGAAAGCTGAAAGATGCACTG-3′
R:
[SEQ ID NO: 26]
5′-ATTTCTAGAGGAGCCACAGAGCATGAGAT-3′
IGF1R 3′UTR primers:
MRE (4600-5514) for miR-192/215
For:
[SEQ ID NO: 27]
5′-ATTTCTAGAATCCATTCACAAGCCTCCTG-3′
Rev:
[SEQ ID NO: 28]
5′-ATTTCTAGA CCTTCCCATCTGTGTCCTTG-3′
MRE (6013-7572) for miR-192/215:
F (4600-5514):
[SEQ ID NO: 29]
5′- ATTTCTAGATTTTGCTGGTCAGCAGTTTG -3′
R (6913-7572):
[SEQ ID NO: 30]
5′- ATTTCTAGATCCATCTGCACAGAAGCAGT-3′
Deletion mutagenesis
IGF1 Deletion
For:
[SEQ ID NO: 31]
5′-TTAATTGACCATACTGGATACTATTTCTGTTCTCTCTTCCCCAA-3′
Rev:
[SEQ ID NO: 32]
5′-TTGGGGAAGAGAGAACAGAAATAGTATCCAGTATGGTCAATTAA-3
IGF1R Deletion
For(4600-5514):
[SEQ ID NO: 33]
5′TGTACACACCCGCCTGACACCATTACAAAAAAACACGTGG3′
Rev(4600-5514):
[SEQ ID NO: 34]
5′CCACGTGTTTTTTTGTAATGGTGTCAGGCGGGTGTGTACA3′
For(6913-7572):
[SEQ ID NO: 35]
5′TTTCTCTGTTCCTAGGACTCTTACAGTTCTATGTTAGACC3′
Rev(6913-7572):
[SEQ ID NO: 36]
5′GGTCTAACATAGAACTGTAAGAGTCCTAGGAACAGAGAAA3′
miR-194-2-192 promoter primers:
(−1871-+186)
F:
[SEQ ID NO: 37]
ATTGAGCTCCCTACGACACAGTGCGAGAGG
R:
[SEQ ID NO: 38]
ACTCTCGAGGGAAACCAAGGCACAGAGGAA
(−1104) F:
[SEQ ID NO: 39]
ATTGAGCTCCAGCCCCCCTCTCAGATCCTC
(−958) F:
[SEQ ID NO: 40]
5′ ATTGAGCTCATCAGGGCACAGGGGGACCCA3′
(−912) F:
[SEQ ID NO: 41]
5′ ATTGAGCTCCTCTGGGCTCTGCCTTGCCCC3′
(−631) F:
[SEQ ID NO: 42]
5′ ATTGAGCTCCCAGCTCCAGCACTTGGAGGG3′
(−530) F:
[SEQ ID NO: 43]
5′ATTGAGCTCATTGCCCCCCACACATCTTGT3′
(−481) F:
[SEQ ID NO: 44]
5′ ATTGAGCTCCCCTGCCCTGCTTCCCAGTG3′
(−429) F:
[SEQ ID NO: 45]
5′ ATTGAGCTC GAAACCAAGGCTCGGGTTGGG3′
(−339) F:
[SEQ ID NO: 46]
5′ ATTGAGCTCGTGGGGGAGATCTGGGTACTG3′
(−245) F:
[SEQ ID NO: 47]
5′ ATTGAGCTCGGACAGCTGGGGCAGCAGGCT3′
(−125) F:
[SEQ ID NO: 48]
5′ ATTGAGCTCTCCTGGACCCGCCCCACCCTGC3′
Mutation primers for p53 binding sites
in miR-194-2-192 cluster promoter
192-Exch1 (F)
[SEQ ID NO: 49]
5′-CCAGCCTGATGCTTCCTGGATCCTCCCCACCCTGCCCGGGCACA-3′
192-Exch1 (R)
[SEQ ID NO: 50]
5′-TGTGCCCGGGCAGGGTGGGGAGGATCCAGGAAGCATCAGGCTGG-3′
192-Exch2 (F)
[SEQ ID NO: 51]
5′-GCTTCCTGGACCCGCCCCACTCTTCCCGGGCACAGTCCAGGGCT-3′
192-Exch2(R)
[SEQ ID NO: 52]
5′-AGCCCTGGACTGTGCCCGGGAAGAGTGGGGCGGGTCCAGGAAGC-3′
192-Exch3 (F)
[SEQ ID NO: 53]
5′-GCTTCCTGGATCCTCCCCACTCTTCCCGGGCACAGTCCAGGGCT-3′
192-Exch3 (R)
[SEQ ID NO: 54]
5′-AGCCCTGGACTGTGCCCGGGAAGAGTGGGGAGGATCCAGGAAGC-3′
[SEQ ID NO: 55]
FIG. 2B
[SEQ ID NO: 56]
FIG. 6D - miR-192
[SEQ ID NO: 57]
FIG. 6D - IGF-13′UTR
[SEQ ID NO: 58]
FIG. 6D - miR-215
[SEQ ID NO: 59]
FIG. 6E - IGF-R 3′ UTR (top listed sequence)
[SEQ ID NO: 60]
FIG. 6E - IGF-R 3′ UTR (bottom listed sequence)
[SEQ ID NO: 61]
FIG. 18B - top listed sequence
[SEQ ID NO: 62]
FIG. 18B - bottom listed sequence
[SEQ ID NO: 63]
FIG. 18C - top listed sequence
[SEQ ID NO: 64]
FIG. 18C - bottom listed sequence
[SEQ ID NO: 65]
FIG. 18D - top listed sequence
[SEQ ID NO: 66]
FIG. 18D - bottom listed sequence
[SEQ ID NO: 67]
FIG. 18E - top listed sequence
[SEQ ID NO: 68]
FIG. 18E - bottom listed sequence
[SEQ ID NO: 69]
FIG. 20B - top listed sequence
[SEQ ID NO: 70]
FIG. 20B - bottom listed sequence
[SEQ ID NO: 71]
FIG. 20C - top listed sequence
[SEQ ID NO: 72]
FIG. 20C - bottom listed sequence

Method of Treating Cancer Subjects

This example describes a method of selecting and treating subjects that are likely to have a favorable response to treatments with compositions herein.

A subject diagnosed with cancer ordinarily first undergoes tissue resection with an intent to cure. Tumor samples are obtained from the portion of the tissue removed from the subject. RNA is then isolated from the tissue samples using any appropriate method for extraction of small RNAs that are well known in the art, such as by using TRIZOL™. Purified RNA is then subjected to RT-PCR using primers specific miR/s or other differentially expressed miRNAs disclosed, optionally in conjunction with genetic analysis. These assays are run to determine the expression level of the pertinent RNA in the tumor. If differentially expressed miR expression pattern is determined, especially if mutant status is ascertained, the subject is a candidate for treatment with the compositions herein.

Accordingly, the subject is treated with a therapeutically effective amount of the compositions according to methods known in the art. The dose and dosing regimen of the compositions will vary depending on a variety of factors, such as health status of the subject and the stage of the cancer. Typically, treatment is administered in many doses over time.

Methods of Diagnosing Cancer Subjects

In one particular aspect, there is provided herein a method of diagnosing whether a subject has, or is at risk for developing, cancer. The method generally includes measuring the differential miR expression pattern of the miR/s compared to control. In certain embodiments, the level of the at least one gene product is measured using Northern blot analysis. Also, in certain embodiments, the level of the at least one gene product in the test sample is less than the level of the corresponding miR gene product expression in the control sample, and/or the level of the at least one miR gene product expression in the test sample is greater than the level of the corresponding miR gene product expression in the control sample.

Measuring miR Gene Products

The level of the at least one miR gene product can be measured by reverse transcribing RNA from a test sample obtained from the subject to provide a set of target oligodeoxynucleotides; hybridizing the target oligodeoxynucleotides to a microarray comprising miRNA-specific probe oligonucleotides to provide a hybridization profile for the test sample; and, comparing the test sample hybridization profile to a hybridization profile generated from a control sample. An alteration in the signal of at least one miRNA is indicative of the subject either having, or being at risk for developing, cancer.

Array Preparation and Screening

Also provided herein are the preparation and use of miRNA arrays, which are ordered macroarrays or microarrays of nucleic acid molecules (probes) that are fully or nearly complementary or identical to a plurality of miRNA molecules or precursor miRNA molecules and that are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters.

Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support; in contrast to the nitrocellulose-based material of filter arrays. By having an ordered array of miRNA-complementing nucleic acid samples, the position of each sample can be tracked and linked to the original sample.

A variety of different array devices in which a plurality of distinct nucleic acid probes are stably associated with the surface of a solid support are known to those of skill in the art. Useful substrates for arrays include nylon, glass and silicon. The arrays may vary in a number of different ways, including average probe length, sequence or types of probes, nature of bond between the probe and the array surface, e.g., covalent or non-covalent, and the like. The labeling and screening methods described herein and the arrays are not limited in its utility with respect to any parameter except that the probes detect miRNA; consequently, methods and compositions may be used with a variety of different types of miRNA arrays.

Diagnostic and Therapeutic Applications

In another aspect, there is provided herein are methods of treating a cancer in a subject, where the signal of at least one miRNA, relative to the signal generated from the control sample, is de-regulated (e.g., down-regulated and/or up-regulated).

Also provided herein are methods of diagnosing whether a subject has, or is at risk for developing, a cancer associated with one or more adverse prognostic markers in a subject, by reverse transcribing RNA from a test sample obtained from the subject to provide a set of target oligodeoxynucleotides; hybridizing the target oligodeoxynucleotides to a microarray comprising miRNA-specific probe oligonucleotides to provide a hybridization profile for the test sample; and, comparing the test sample hybridization profile to a hybridization profile generated from a control sample. An alteration in the signal is indicative of the subject either having, or being at risk for developing, the cancer.

Therapeutic/Prophylactic Methods and Compositions

The invention provides methods of treatment and prophylaxis by administration to a subject an effective amount of a miR, with or without combination therapy. In a preferred aspect, the therapeutic is substantially purified. The subject is preferably an animal, including but not limited to, animals such as cows, pigs, chickens, etc., and is preferably a mammal, and most preferably human.

Various delivery systems are known and are used to administer a therapeutic of the invention, e.g., encapsulation in liposomes, microparticles, microcapsules, expression by recombinant cells, receptor-mediated endocytosis, construction of a therapeutic nucleic acid as part of a retroviral or other vector, etc. Methods of introduction include, but are not limited to, intradermal, intramuscular, intraperitoneal, intravenous, subcutaneous, intranasal, and oral routes. The compounds are administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir.

In a specific embodiment, it may be desirable to administer the pharmaceutical compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, the implant being of a porous, non-porous, or gelatinous material, including membranes, such as sialastic membranes, or fibers. In one embodiment, administration is by direct injection at the site (or former site) of a malignant tumor or neoplastic or pre-neoplastic tissue.

In a specific embodiment where the therapeutic is a nucleic acid encoding a protein therapeutic the nucleic acid is administered in vivo to promote expression of its encoded protein, by constructing it as part of an appropriate nucleic acid expression vector and administering it so that it becomes intracellular, or coating with lipids or cell-surface receptors or transfecting agents, or by administering it in linkage to a homeobox-like peptide which is known to enter the nucleus. Alternatively, a nucleic acid therapeutic can be introduced intracellularly and incorporated within host cell DNA for expression, by homologous recombination.

The present invention also provides pharmaceutical compositions. Such compositions comprise a therapeutically effective amount of a therapeutic, and a pharmaceutically acceptable carrier or excipient. Such a carrier includes, but is not limited to, saline, buffered saline, dextrose, water, glycerol, ethanol, and combinations thereof. The carrier and composition can be sterile. The formulation will suit the mode of administration.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition also includes a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it is be dispensed with an infusion bottle containing sterile pharmaceutical grade water or saline. Where the composition is administered by injection, an ampoule of sterile water for injection or saline is provided so that the ingredients are mixed prior to administration.

The therapeutics of the invention can be formulated as neutral or salt forms.

Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the therapeutic of the invention which will be effective in the treatment of a particular disorder or condition will depend on the nature of the disorder or condition, and is determined by standard clinical techniques. In addition, in vitro assays may optionally be employed to help identify optimal dosage ranges. The precise dose to be employed in the formulation will also depend on the route of administration, and the seriousness of the disease or disorder, and is decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems

The invention also provides a pharmaceutical pack or kit comprising one or more containers filled with one or more of the ingredients of the pharmaceutical compositions of the invention. Optionally associated with such container(s) is a notice in the form prescribed by a governmental agency regulating the manufacture, use or sale of pharmaceuticals or biological products, which notice reflects approval by the agency of manufacture, use or sale for human administration.

Administration

Simultaneous administration may, e.g., take place in the form of one fixed combination with two or more active ingredients, or by simultaneously administering two or more active ingredients that are formulated independently.

Sequential use (administration) preferably means administration of one (or more) components of a combination at one time point, other components at a different time point, that is, in a chronically staggered manner, preferably such that the combination shows more efficiency than the single compounds administered independently (especially showing synergism).

Separate use (administration) preferably means administration of the components of the combination independently of each other at different time points, preferably meaning that the components (a) and (b) are administered such that no overlap of measurable blood levels of both compounds are present in an overlapping manner (at the same time).

Also combinations of two or more of sequential, separate and simultaneous administration are possible, preferably such that the combination component-drugs show a joint therapeutic effect that exceeds the effect found when the combination component-drugs are used independently at time intervals so large that no mutual effect on their therapeutic efficiency can be found, a synergistic effect being especially preferred.

The term “delay of progression” as used herein means administration of the combination to subjects being in a pre-stage or in an early phase, of the first manifestation or a relapse of the disease to be treated, in which subjects, e.g., a pre-form of the corresponding disease is diagnosed or which subjects are in a condition, e.g., during a medical treatment or a condition resulting from an accident, under which it is likely that a corresponding disease will develop.

“Jointly therapeutically active” or “joint therapeutic effect” means that the compounds may be given separately (in a chronically staggered manner, especially a sequence-specific manner) in such time intervals that they preferably, in the warm-blooded animal, especially human, to be treated, still show a (preferably synergistic) interaction (joint therapeutic effect). Whether this is the case, can inter alia be determined by following the blood levels, showing that both compounds are present in the blood of the human to be treated at least during certain time intervals.

“Pharmaceutically effective” preferably relates to an amount that is therapeutically or in a broader sense also prophylactically effective against the progression of a proliferative disease.

Kits

Any of the compositions described herein may be comprised in a kit. In a non-limiting example, reagents for isolating miRNA, labeling miRNA, and/or evaluating an miRNA population using an array are included in a kit. The kit may further include reagents for creating or synthesizing miRNA probes. The kits will thus comprise, in suitable coniainer means, an enzyme for labeling the miRNA by incorporating labeled nucleotide or unlabeled nucleotides that are subsequently labeled. It may also include one or more buffers, such as reaction buffer, labeling buffer, washing buffer, or a hybridization buffer, compounds for preparing the miRNA probes, and components for isolating miRNA. Other kits may include components for making a nucleic acid array comprising oligonucleotides complementary to miRNAs, and thus, may include, for example, a solid support.

For any kit embodiment, including an array, there can be nucleic acid molecules that contain a sequence that is identical or complementary to all or part of any of the sequences herein.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However; various combinations of components may be comprised in a vial. The kits of the present invention also will typically include a means for containing the nucleic acids, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being one preferred solution. Other solutions that may be included in a kit are those solutions involved in isolating and/or enriching miRNA from a mixed sample.

However, the components of the kit may be provided as dried powder(s). When reagents and/or components are provided as a dry powder, the powder can be reconstituted by the addition of a suitable solvent. It is envisioned that the solvent may also be provided in another container means. The kits may also include components that facilitate isolation of the labeled miRNA. It may also include components that preserve or maintain the miRNA or that protect against its degradation. The components may be RNAse-free or protect against RNAses.

Also, the kits can generally comprise, in suitable means, distinct containers for each individual reagent or solution. The kit can also include instructions for employing the kit components as well the use of any other reagent not included in the kit. Instructions may include variations that can be implemented. It is contemplated that such reagents are embodiments of kits of the invention. Also, the kits are not limited to the particular items identified above and may include any reagent used for the manipulation or characterization of miRNA.

It is also contemplated that any embodiment discussed in the context of an miRNA array may be employed more generally in screening or profiling methods or kits of the invention. In other words, any embodiments describing what may be included in a particular array can be practiced in the context of miRNA profiling more generally and need not involve an array per se.

It is also contemplated that any kit, array or other detection technique or tool, or any method can involve profiling for any of these miRNAs. Also, it is contemplated that any embodiment discussed in the context of an miRNA array can be implemented with or without the array format in methods of the invention; in other words, any miRNA in an miRNA array may be screened or evaluated in any method of the invention according to any techniques known to those of skill in the art. The array format is not required for the screening and diagnostic methods to be implemented.

The kits for using miRNA arrays for therapeutic, prognostic, or diagnostic applications and such uses are contemplated by the inventors herein. The kits can include an miRNA array, as well as information regarding a standard or normalized miRNA profile for the miRNAs on the array. Also, in certain embodiments, control RNA or DNA can be included in the kit. The control RNA can be miRNA that can be used as a positive control for labeling and/or array analysis.

The methods and kits of the current teachings have been described broadly and generically herein. Each of the narrower species and sub-generic groupings falling within the generic disclosure also form part of the current teachings. This includes the generic description of the current teachings with a proviso or negative limitation removing any subject matter from the genus, regardless of whether or not the excised material is specifically recited herein.

Commercial Package

Commercial package or a product, as used herein defines especially a “kit of parts” in the sense that the components (a) and (b) as defined above can be dosed independently or by use of different fixed combinations with distinguished amounts of the components (a) and (b), i.e., simultaneously or at different time points. Moreover, these terms comprise a commercial package comprising (especially combining) as active ingredients components (a) and (b), together with instructions for simultaneous, sequential (chronically staggered, in time-specific sequence, preferentially) or (less preferably) separate use thereof in the delay of progression or treatment of a proliferative disease. The parts of the kit of parts can then, e.g., be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the combination partners (a) and (b) (as can be determined according to standard methods. The ratio of the total amounts of the combination partner (a) to the combination partner (b) to be administered in the combined preparation can be varied, e.g., in order to cope with the needs of a subject sub-population to be treated or the needs of the single subject which different needs can be due to the particular disease, age, sex, body weight, etc. of the subjects. Preferably, there is at least one beneficial effect, e.g., a mutual enhancing of the effect of the combination partners (a) and (b), in particular a more than additive effect, which hence could be achieved with lower doses of each of the combined drugs, respectively, than tolerable in the case of treatment with the individual drugs only without combination, producing additional advantageous effects, e.g., less side effects or a combined therapeutic effect in a non-effective dosage of one or both of the combination partners (components) (a) and (b), and very preferably a strong synergism of the combination partners (a) and (b).

Both in the case of the use of the combination of components (a) and (b) and of the commercial package, any combination of simultaneous, sequential and separate use is also possible, meaning that the components (a) and (b) may be administered at one time point simultaneously, followed by administration of only one component with lower host toxicity either chronically, e.g., more than 3-4 weeks of daily dosing, at a later time point and subsequently the other component or the combination of both components at a still later time point (in subsequent drug combination treatment courses for an optimal antitumor effect) or the like.

The combination of the invention can also be applied in combination with other treatments, e.g., surgical intervention, hyperthermia and/or irradiation therapy.

Pharmaceutical Compositions & Preparations

The pharmaceutical compositions according to the present invention can be prepared by conventional means and are those suitable for enteral, such as oral or rectal, and parenteral administration to mammals including man, comprising a therapeutically effective amount of a microtubule active agent and at least one pharmaceutically active agent alone or in combination with one or more pharmaceutically acceptable carriers, especially those suitable for enteral or parenteral application.

The pharmaceutical compositions comprise from about 0.00002 to about 100%, especially, e.g., in the case of infusion dilutions that are ready for use) of 0.0001 to 0.02%, or, e.g., in case of injection or infusion concentrates or especially parenteral formulations, from about 0.1% to about 95%, preferably from about 1% to about 90%, more preferably from about 20% to about 60%-DISCUSS active ingredient (weight by weight, in each case). Pharmaceutical compositions according to the invention may be, e.g., in unit dose form, such as in the form of ampoules, vials, dragees, tablets, infusion bags or capsules.

The effective dosage of each of the combination partners employed in a formulation of the present invention may vary depending on the particular compound or pharmaceutical compositions employed, the mode of administration, the condition being treated and the severity of the condition being treated. A physician, clinician or veterinarian of ordinary skill can readily determine the effective amount of each of the active ingredients necessary to prevent, treat or inhibit the progress of the condition.

Pharmaceutical preparations for the combination therapy for enteral or parenteral administration are, e.g., those in unit dosage forms, such as sugar-coated tablets, capsules or suppositories, and furthermore ampoules. If not indicated otherwise, these formulations are prepared by conventional means, e.g., by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units. One of skill in the art has the ability to determine appropriate pharmaceutically effective amounts of the combination components.

Preferably, the compounds or the pharmaceutically acceptable salts thereof, are administered as an oral pharmaceutical formulation in the form of a tablet, capsule or syrup; or as parenteral injections if appropriate.

In preparing compositions for oral administration, any pharmaceutically acceptable media may be employed such as water, glycols, oils, alcohols, flavoring agents, preservatives, coloring agents. Pharmaceutically acceptable carriers include starches, sugars, microcrystalline celluloses, diluents, granulating agents, lubricants, binders, disintegrating agents.

Solutions of the active ingredient, and also suspensions, and especially isotonic aqueous solutions or suspensions, are useful for parenteral administration of the active ingredient, it being possible, e.g., in the case of lyophilized compositions that comprise the active ingredient alone or together with a pharmaceutically acceptable carrier, e.g., mannitol, for such solutions or suspensions to be produced prior to use. The pharmaceutical compositions may be sterilized and/or may comprise excipients, e.g., preservatives, stabilizers, wetting and/or emulsifying agents, solubilizers, salts for regulating the osmotic pressure and/or buffers, and are prepared in a manner known per se, e.g., by means of conventional dissolving or lyophilizing processes. The solutions or suspensions may comprise viscosity-increasing substances, such as sodium carboxymethylcellulose, carboxymethylcellulose, dextran, polyvinylpyrrolidone or gelatin. Suspensions in oil comprise as the oil component the vegetable, synthetic or semi-synthetic oils customary for injection purposes. The isotonic agent may be selected from any of those known in the art, e.g. mannitol, dextrose, glucose and sodium chloride. The infusion formulation may be diluted with the aqueous medium. The amount of aqueous medium employed as a diluent is chosen according to the desired concentration of active ingredient in the infusion solution. Infusion solutions may contain other excipients commonly employed in formulations to be administered intravenously such as antioxidants.

The present invention further relates to “a combined preparation”, which, as used herein, defines especially a “kit of parts” in the sense that the combination partners (a) and (b) as defined above can be dosed independently or by use of different fixed combinations with distinguished amounts of the combination partners (a) and (b), i.e., simultaneously or at different time points. The parts of the kit of parts can then, e.g., be administered simultaneously or chronologically staggered, that is at different time points and with equal or different time intervals for any part of the kit of parts. The ratio of the total amounts of the combination partner (a) to the combination partner (b) to be administered in the combined preparation can be varied, e.g., in order to cope with the needs of a subject sub-population to be treated or the needs of the single subject based on the severity of any side effects that the subject experiences.

In view of the many possible embodiments to which the principles of the inventors' invention may be applied, it should be recognized that the illustrated embodiments are only preferred examples of the invention and should not be taken as a limitation on the scope of the invention. Rather, the scope of the invention is defined by the following claims. The inventors therefore claim as the inventors' invention all that comes within the scope and spirit of these claims.

Therefore, it is intended that the invention not be limited to the particular embodiment disclosed herein contemplated for carrying out this invention, but that the invention will include all embodiments falling within the scope of the claims.

The publication and other material used herein to illuminate the invention or provide additional details respecting the practice of the invention, are incorporated be reference herein, and for convenience are provided in the following bibliography. Citation of the any of the documents recited herein is not intended as an admission that any of the foregoing is pertinent prior art. All statements as to the date or representation as to the contents of these documents is based on the information available to the applicant and does not constitute any admission as to the correctness of the dates or contents of these documents.

1. A method of treating a disorder mediated by a p53-HDM2 interaction comprising administering to a subject in need thereof a combination of at least one miR gene product and at least one indole inhibitor of human double minute 2 (HDM2), or a pharmaceutically acceptable salt, solvate, or prodrug thereof.

2. The method of claim 1, wherein the disorder is multiple myeloma, and the miR gene product comprises one or more of: miR-192, miR-194 and miR-215.

3. A combination of an indole inhibitor of human double minute 2 (HDM2), or a pharmaceutically acceptable salt, solvate, or prodrug thereof, and one or more of a miR gene product selected from: miR-192, miR0194 and miR-215.

4. The combination of claim 3, wherein the indole inhibitor of human double minute 2 (HDM2) comprises MI-219 having the structure

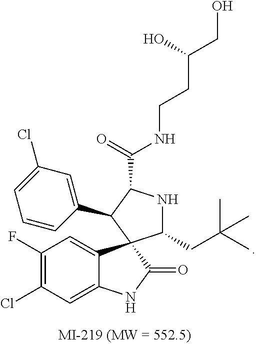 [SMILES: CC(C)(C)C[C@H]1N[C@@H](C(=O)NCC[C@H](O)CO)[C@H](c2cccc(Cl)c2)[C@@]12C(=O)Nc1cc(Cl)c(F)cc12]

5. The combination of claim 3, wherein the indole inhibitor of human double minute 2 (HDM2) comprises Nutlin 3 having the structure

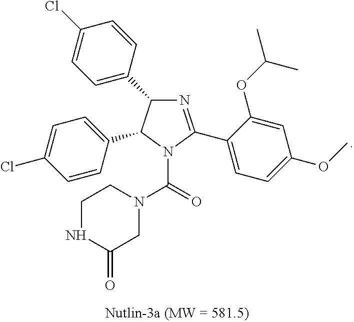 [SMILES: COc1ccc(C2=N[C@@H](c3ccc(Cl)cc3)[C@@H](c3ccc(Cl)cc3)N2C(=O)N2CCNC(=O)C2)c(OC(C)C)c1]

6. A pharmaceutical composition comprising the combination according to claim 3.

7. A commercial package comprising a combination according to claim 3.

8. A commercial package of claim 7, wherein the unit dosage form is a fixed combination.

9. A method of treating a subject comprising:
administering to the subject a therapeutically effective amount of the combination of claim 3, wherein the subject has a hyperproliferative disease.

10. The method of claim 9, wherein the hyperproliferative disease is multiple myeloma.

11. The method of claim 9, wherein cells of the hyperproliferative disease express functional p53.

12. A kit comprising a combination of claim 6, and instructions for administering the compound to a subject having a hyperproliferative disease.

13. The kit of claim 12, wherein the hyperproliferative disease is multiple myeloma.

14. The kit of claim 12, wherein the instructions direct co-administration of the compound together with the one or more anticancer agents.

15. A method of treating a disorder in a subject, comprising administering to said subject a therapeutically effective amount of a combination of claim 3, wherein the disorder is multiple myeloma.

16. The method of claim 15, wherein the indole inhibitor of human double minute 2 (HDM2) is administered prior to the miR gene product.

17. The method of claim 15, wherein the indole inhibitor of human double minute 2 (HDM2) is administered after the miR gene product.

18. The method of claim 15, wherein the indole inhibitor of human double minute 2 (HDM2) is administered concurrently with the miR gene product.

19. A combination of: i) an indole inhibitor of human double minute 2 (HDM2); and ii) a miR gene product comprising one or more of: miR-192, miR-194 and miR-215; for simultaneous, concurrent, separate or sequential use in for preventing or treating a proliferative disease

20. The combination according to claim 19, wherein the indole inhibitor of human double minute 2 (HDM2) comprises MI-219 or of a pharmaceutically acceptable salt, ester or prodrug thereof.

21. The combination according to claim 19, wherein the indole inhibitor of human double minute 2 (HDM2) comprises Nutlin 3 or of a pharmaceutically acceptable salt, ester or prodrug thereof.

22. A pharmaceutical composition comprising the combination of claim 19.

23. A commercial package comprising the combination of claim 19.

24. A commercial package of claim 23, wherein the unit dosage form is a fixed combination.

25. A method of treating in a subject a disorder mediated by a p53-MDM2 interaction comprising administering to the subject a therapeutically effective amount of a pharmaceutical composition comprising a combination of i) an indole inhibitor of human double minute 2 (HDM2); and ii) a miR gene product comprising one or more of: miR-192, miR-194 and miR-215; and a pharmaceutically acceptable carrier.

26. A method for regulating human double minute 2 (HDM2)-p53 auto regulatory loop, in a subject in need thereof, comprising upregulating the expression of one or more of: miR-192, miR-194 and miR-215.

27. A method for increasing the ability of p53 to modulate HDM2 expression in a subject having multiple myeloma (MM), comprising administering an effective amount of a miR gene product comprising one or more of: miR-192, miR-194 and miR-215, sufficient to inhibit expression of HDM2.

28. Use of miR-192, miR-194 and/or miR-215 as mediators in the pharmacological activation of the p53 pathway in multiple myeloma (MM) cells.

29. A method for inhibiting expression of HDM2 mRNA comprising up-modulating expression of one or more of: miR-192, miR-194 and miR-215.

30. A composition for inhibiting cell growth and enhancing apoptosis in multiple myeloma cells, comprising a gene product comprising one or more of: miR-192, miR-194 and miR-215.

31. The composition of claim 30, further including one or more HDM2 inhibitors.

32. The composition of claim 31, wherein the HDM2 inhibitor comprises MI-219.

33. The composition of claim 31, wherein the HDM2 inhibitor comprises Nutlin 3a.

34. A method for inhibiting cell growth and enhancing apoptosis in multiple myeloma (MM) cells, comprising administering:
an effective amount of one or more miR gene products that affect proliferation rate in MM cells and/or the homing and migration ability of MM cells,
wherein the miR gene products comprises one or more of: miR-192, miR-194 and miR-215.

35. The method of claim 34, further including administering one or more p53 pharmacological activators in an amount sufficient to cause HDM2 down-regulation, and/or one or more of: p53, p21, Puma up-regulation.

36. A method of treating multiple myeloma (MM) in a subject who has a MM in which at least one miR gene product is down-regulated in the MM cells of the subject relative to control cells, comprising:
administering to the subject an effective amount of at least one isolated miR gene product, wherein the miR gene product comprises one or more of: miR-192, miR-194 and miR-215, such that proliferation of MM cells in the subject is inhibited.

37. The method of claim 36, further including administering an effective amount of a p53 pharmacological activator.

38. The method of claim 37, wherein the p53 pharmacological activator comprises one or more of: MI-219 and Nutlin 3.

39. A pharmaceutical composition for treating MM, comprising at least one isolated miR gene product and a pharmaceutically-acceptable carrier, wherein the at least one isolated miR gene product corresponds to a miR gene product that is down-regulated in MM cells relative to suitable control cells, wherein the isolated miR gene product comprises one or more of: miR-192, miR194 and miR-215.

40. A method of diagnosing multiple myeloma, comprising detecting a decreased amount of one or more of: miR-192, miR-194 and miR-215 gene product as compared to a control.

41. A method of identifying an anti-MM agent, comprising providing a test agent to a cell and measuring the level of at least one miR gene product associated with decreased expression levels in MM cells, wherein an increase in the level of the miR gene product in the cell, relative to a suitable control cell, is indicative of the test agent being an anti-MM agent.

42. The method of claim 40 wherein the method is performed to distinguish MM from monoclonal gammopathy of undetermined significance (MGUS).
